(19) United States
(12) Patent Application Publication
(10) Pub. No.: US 2016/0326597 A1
(43) Pub. Date: Nov. 10, 2016

(54) DETERMINANTS OF CANCER RESPONSE TO IMMUNOTHERAPY

(71) Applicant: MEMORIAL SLOAN KETTERING CANCER CENTER, New York, NY (US)

(72) Inventors: Timothy Chan, Cortlandt Manor, NY (US); Jedd Wolchok, New York, NY (US); Vladimir Makarov, New York, NY (US); Alexandra Snyder Charen, New York, NY (US)

(21) Appl. No.: 15/109,464

(22) PCT Filed: Dec. 23, 2014
(86) PCT No.: PCT/US14/72125

§ 371 Date: Jul. 1, 2016

Related U.S. Application Data
(60) Provisional application No. 61/923,183, filed on Jan. 2, 2014.
(60) Provisional application No. 62/066,034, filed on Oct. 20, 2014.
(60) Provisional application No. 62/072,893, filed on Oct. 30, 2014.

(51) Int. Cl. C12Q 1/68; C07K 16/28

(52) U.S. Cl. CPC C12Q 1/6886; C07K 16/2896; C12Q 2600/106; C12Q 2600/156

(57) ABSTRACT

Molecular determinants of cancer response to immunotherapy are described, as are systems and tools for identifying and/or characterizing cancers likely to respond to immunotherapy. The present invention encompasses the discovery that the likelihood of a favorable response to cancer immunotherapy can be predicted. The present invention further comprises the discovery that cancer cells may harbor somatic mutations that result in neoepitopes that are recognizable by a patient's immune system as non-self. The identification of one or more neoepitopes in a cancer sample is useful for determining which cancer patients are likely to respond favorably to immunotherapy, in particular, treatment with an immune checkpoint modulator.

### CROSS REFERENCE TO RELATED APPLICATIONS

This application claims priority to each of U.S. Provisional Patent Application Ser. No. 61/923,183, filed Jan. 2, 2014; U.S. Provisional Patent Application Ser. No. 62/066,034, filed Oct. 20, 2014; and U.S. Provisional Patent Application Ser. No. 62/072,893, filed Oct. 30, 2014, the entire contents of each of which are hereby incorporated by reference.

### BACKGROUND

Cancer immunotherapy involves the attack of cancer cells by a patient's immune system. Regulation and activation of T lymphocytes depends on signaling by the T cell receptor and also cosignaling receptors that deliver positive or negative signals for activation. Immune responses by T cells are controlled by a balance of costimulatory and inhibitory signals, called immune checkpoints.

Immunotherapy with immune checkpoint inhibitors is revolutionizing cancer therapy. For example, in certain melanoma patients, anti-CTLA4 and anti-PD1 antibodies have offered a remarkable opportunity for long-term disease control in the metastatic setting.

### SUMMARY

The present invention encompasses the discovery that the likelihood of a favorable response to cancer immunotherapy can be predicted. The present invention further comprises the discovery that cancer cells may harbor somatic mutations that result in neoepitopes that are recognizable by a patient's immune system as non-self. The identification of one or more neoepitopes in a cancer sample is useful for determining which cancer patients are likely to respond favorably to immunotherapy, in particular, treatment with an immune checkpoint modulator.

In some embodiments, the invention provides methods for identifying a subject as likely to respond to treatment with an immune checkpoint modulator.

In some embodiments, the methods comprise steps of detecting a somatic mutation in a cancer sample from a subject and identifying the subject as a candidate for treatment with an immune checkpoint modulator. In some embodiments, a subject is identified as likely to respond favorably to treatment with an immune checkpoint modulator.

In some embodiments, detecting a somatic mutation comprises sequencing one or more exomes from a cancer sample. In some embodiments, a somatic mutation comprises a neoepitope recognized by a T cell.

In some embodiments, a neoepitope has greater binding affinity to a major histocompatibility complex (MHC) molecule compared to a corresponding epitope that does not have a mutation.

In some embodiments, a somatic mutation comprises a neoepitope comprising a tetramer that is not expressed in the same cell type that does not have a somatic mutation.

In some embodiments, a neoepitope shares a consensus sequence with an infectious agent. In some embodiments, a neoepitope shares a consensus sequence with a bacterium. In some embodiments, a neoepitope shares a consensus sequence with a virus.

In some embodiments, a somatic mutation comprises a neoepitope comprising a tetramer of Table 1.

In some embodiments, a cancer sample is or comprises melanoma.

In some embodiments, an immune checkpoint modulator interacts with one or more of cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), a killer immunoglobulin-like receptor (KIR), T cell immunoglobulin and mucin domain-containing protein 3 (TIM-3), inducible T cell costimulator (ICOS), CD27, CD28, CD40, CD137, or combinations thereof.

In some embodiments, an immune checkpoint modulator is or comprises an antibody or antigen binding fragment. In some embodiments, an immune checkpoint modulator is ipilumimab. In some embodiments,an immune checkpoint modulator is or comprises tremelimumab. In some embodiments, an immune checkpoint modulator is or comprises nivolumab. In some embodiments, an immune checkpoint modulator is or comprises lambrolizumab. In some embodiments, an immune checkpoint modulator is or comprises pembrolizumab.

In some embodiments, the invention provides methods for identifying a subject as likely to respond to treatment with an immune checkpoint modulator. In some embodiments, the invention provides methods for identifying a subject as likely to respond to treatment with an immune checkpoint modulator, wherein the subject has not previously been treated with a cancer immunotherapeutic.

In some embodiments, the invention provides methods for detecting a somatic mutation in a cancer sample from a subject and identifying the subject as a poor candidate for treatment with an immune checkpoint modulator.

In some embodiments, the invention provides methods for identifying a subject as likely to suffer one or more autoimmune complications if administered an immune checkpoint modulator.

In some embodiments, an autoimmune complication is or comprises enterocolitis, hepatitis, dermatitis (including toxic epidermal necrolysis), neuropathy, and/or endocrinopathy. In some embodiments, an autoimmune complication is or comprises hypothyroidism.

In some embodiments, the invention provides methods for determining that a subject has a cancer comprising a somatic mutation, wherein the somatic mutation comprises a neoepitope comprising a tetramer from Table 1, and selecting for the subject a cancer treatment comprising an immune checkpoint modulator.

In some embodiments, the invention provides methods for treating a subject with an immune checkpoint modulator wherein the subject has previously been identified to have a cancer with one or more somatic mutations, wherein the one or more somatic mutations comprises a neoepitope recognized by a T cell.

In some embodiments, the invention provides methods for improving efficacy of cancer therapy with an immune checkpoint modulator, comprising a step of selecting for receipt of the therapy a subject identified as having a cancer with one or more somatic mutations comprising a neoepitope recognized by a T cell.

In some embodiments, the invention provides improvements to methods of treating cancer by administering immune checkpoint modulators, wherein an improvement comprises administering therapy to a subject identified as having a cancer with one or more somatic mutations comprising a neoepitope recognized by a T cell. In some embodiments, long term clinical benefit is observed after CTLA-4 blockade (e.g., via ipilimumab or tremelimumab) treatment.

In some embodiments, the invention provides methods for treating a cancer selected from the group consisting of carcinoma, sarcoma, myeloma, leukemia, or lymphoma, the methods comprising a step of administering immune checkpoint modulator therapy to a subject identified as having a cancer with one or more somatic mutations comprising a neoepitope recognized by a T cell. In some embodiments, the cancer is a melanoma. In some embodiments, the cancer is a non-small-cell lung carcinoma (NSCLC).

### BRIEF DESCRIPTION OF THE DRAWING

The following figures are presented for the purpose of illustration only, and are not intended to be limiting.

FIG. 1 (comprised of FIGS. 1A-1C) shows paired pre- and post-treatment scans from patients with long-term clinical benefit from therapy (FIG. 1A, Jan. 2, 2011 and Aug. 26, 2013); (FIG. 1B, Sep. 6, 2011 and Jan. 14, 2013) and no benefit/progressive disease (FIG. 1C, Aug. 13, 2009 and Jan. 9, 2010).

FIG. 2 (comprised of FIGS. 2A-2I) shows mutational landscape of tumors from patients with differing clinical benefit from ipilimumab treatment. FIG. 2A shows the mutational load (number of nonsynonymous mutations per exome) categorized by clinical benefit. FIG. 2B shows relationship between mutational load and benefit from ipilimumab. LB, long-term clinical benefit group; NB, minimal or no benefit group; p=0.01 (Mann-Whitney 2-tailed t-test comparing medians for difference in median mutational load between patients with and without long-term clinical benefit). FIG. 2C shows the rate of transitions (Ti) and transversions (Tv) by clinical subgroup. FIG. 2D shows the nucleotide changes in the discovery and validation sets. Mutational spectrum is consistent with previous melanoma genome studies.19 FIG. 2E depicts the Kaplan-Meier curve of overall survival for patients with greater or less than 100 nonsynonymous coding mutations per exome (p=0.041 by Log-Rank test) in the discovery set. FIG. 2F shows the relationship between mutational load and benefit from ipilimumab. LB, long-term clinical benefit group; NB, minimal or no benefit group; p=0.01 (Mann-Whitney 2-tailed t-test comparing medians for difference in median mutational load between patients with and without long-term clinical benefit). FIG. 2G depicts the Kaplan-Meier curve of overall survival for patients with greater or less than 100 nonsynonymous coding mutations per exome (p=0.041 by Log-Rank test) in the discovery set. FIG. 2H depicts the Kaplan-Meier curve of overall survival for patients with greater or less than 100 nonsynonymous coding mutations per exome (p=0.010 by Log-Rank test) in the validation set. FIG. 2I shows the rate of transitions (Ti) and transversions (Tv) by clinical subgroup.

FIG. 3 (comprised of FIGS. 3A-3H) shows that a neoepitope signature defines clinical benefit to ipilimumab. Candidate neoepitopes were identified by mutational analysis as described in the Supplementary Methods. FIG. 3A shows a heat map of candidate tetrapeptide neoepitopes shared by patients with long-term clinical benefit (LB) or with minimal or no clinical benefit (NB) in the discovery set (n=25). Each row represents a neoepitope. The red line indicates the tetrapeptide signature associated with response. The exact tetrapeptides, chromosomal loci, and wild type and mutant nonamers in which they occur are listed in Table 4 and FIG. 19. FIG. 3B shows the same information for the validation set (n=15). FIG. 3C shows the Kaplan-Meier curve for the discovery set, by neoepitope signature positive (blue line) or negative (red line), excluding isolated non-responding tumors. P<0.0001 by Log-Rank test for patients with the signature versus those without. FIG. 3D shows the same data for the validation set. p=0.049 by Log-Rank. FIG. 3E shows a heat map of candidate tetrapeptide neoepitopes shared by patients with long-term clinical benefit (LB) or with minimal or no clinical benefit (NB) in the discovery set (n=25). Each row represents a neoepitope. The red line indicates the tetrapeptide signature associated with response. The exact tetrapeptides, chromosomal loci, and wild type and mutant nonamers in which they occur are listed in Table 4 and FIG. 19. FIG. 3F shows the same information for the validation set (n=15). FIG. 3G shows the Kaplan-Meier curve for the discovery set, by neoepitope signature positive (blue line) or negative (red line), excluding isolated non-responding tumors. P<0.0001 by Log-Rank test for patients with the signature versus those without. FIG. 3H shows the same data for the validation set. p=0.049 by Log-Rank.

FIG. 4 (comprised of FIGS. 4A-4F) shows neoepitopes activate T cells from ipilimumab-treated patients. FIG. 4A illustrates the diversity of neoepitope generation as function of genomic location. Neoepitopes from three representative LB patients are plotted as a function of genomic location. The candidate neoepitopes in the signature can be generated by different genes. Chromosomal locations of neoepitopes are plotted along the x-axis. Height of peak indicates how many patients share that amino acid sequence in the discovery and validation sets. FIG. 4B shows an example tetrapeptide substring of Toxoplasma gondii. In each case, the nonamer containing the mutation is predicted to bind and be presented by a patient-specific HLA. FIG. 4C shows the polyfunctional T cell response to TESPFEQHI versus wild type peptide TKSPFEQHI. FIG. 4D shows the dual positive (IFN-γ and TNF-α) CD8+ T cell response to TESPFEQHI versus wild type peptide TKSPFEQHI and the increase in IFN-γ+ T cells over time. FIG. 4E shows the dual positive (IFN-γ and TNF-α) CD8+ T cell response to GLEREGFTF versus wild type peptide GLERGGFTF and illustrates the increase in peptide-specific T cells 24 weeks after initiation of treatment with ipilimumab relative to baseline. FIG. 4F shows an example tetrapeptide substring of human cytomegalovirus immediate early epitope. In each case, the nonamer containing the mutation is predicted to bind and be presented by a patient-specific HLA.

FIG. 5 shows an analysis pipeline for the discovery set in which mutations with coverage less than or equal to 10× were excluded, and candidates with coverage less than 35× were manually reviewed using the integrated genomics viewer (IGV).

FIG. 6 (comprised of FIGS. 6A-6D) shows a representative list of the most commonly mutated genes in each clinical subgroup. Candidate mutations were validated by an orthogonal sequencing method such as Ion Torrent sequencing or MiSeq. FIG. 6A depicts a representative list of the recurrently mutated genes in the discovery and validation sets. FIG. 6B depicts the distribution of mutation types across samples in the discovery and validation sets. FIG. 6C depicts a representative list of the recurrently mutated genes in the discovery and validation sets. FIG. 6D depicts the distribution of mutation types across samples in the discovery and validation sets.

FIG. 7 (comprised of FIGS. 7A-7F) shows the drivers and mutational loads for long-term benefit and minimal or no benefit patients. FIG. 7A shows the occurrence of mutations in known melonam driver genes in tumors of each clinical group in the discovery set. FIG. 7B depicts mutations in known melanoma driver genes in tumors of each clinical group in the validation set. FIG. 7C shows the number of exonic missense mutations per sample in the validation set. FIG. 7D shows a comparison of median exonic missense mutations per sample in the validation set. FIG. 7E depicts the mutational loads of patient subgroups with no radiographic evidence of disease (NED), disease control for greater than 6 months (ongoing in all but one patient), disease control for less than 6 months, and no response (NR). P=0.03 for difference between patients with NED and those with no response (Mann-Whitney 2-tailed t-test comparing medians). FIG. 7F depicts the mutational loads of patient subgroups with no radiographic evidence of disease (NED), disease control for greater than 6 months (ongoing in all but one patient), disease control for less than 6 months, and no response (NR). P=0.03 for difference between patients with NED and those with no response (Mann-Whitney 2-tailed t-test comparing medians).

FIG. 8 shows a neoepitope analysis pipeline. All steps are executed for predicted wild type and mutant. MHC Class I prediction is by NetMHCv3.4 and/or RANKPEP. T cell immunogenicity prediction by IEDB program that masks HLA-specific amino acids (http://tools.immunepitope.org/immunogenicity/).

FIG. 9 (comprised of FIGS. 9A-9C) shows representative scans from patients in the discovery set pre- and post-treament. FIG. 9A shows two sites from one patient (May 1, 2008 and May 30, 2013) with no radiographic evidence of disease. FIG. 9B shows scans from patients with clinical benefit of greater than 6 months. Top is from Sep. 6, 2011 and Jan. 14, 2013. Bottom is from Sep. 19, 2007 and Jan. 15, 2009. FIG. 9C shows scans from fTom patients with no response to therapy. Top is May 27, 2010 and Dec. 21, 2010. Bottom is Mar. 3, 2011 and Nov. 18, 2011.

FIG. 10 (comprised of FIGS. 10A-10K) shows peptide analyses, discovery and validation. FIG. 10A shows across all samples in the discovery set, the mutant peptide is more likely to bind MHC Class I than the corresponding wild type peptide. FIG. 10B shows across all samples in the validation set, the mutant peptide is more likely to bind MHC Class I than the corresponding wild type peptide. FIGS. 10C and 10D show the frequency of amino acids in common tetrapeptides in LB and NB Groups. The height of each letter reflects the frequency of a given amino acid at that position. Phenylalanine (F) at positions 3 and 4 are not seen in the NB group. FIG. 10E shows the known antigens of which tetrapeptides comprise substring, by clinical group. Conserved tetrapeptide neoepitopes comprise substrings of antigens from infectious pathogens with evidence in vitro for T cell activation. FIG. 10F shows MART-1 and EKLS substrings. FIG. 10G shows across all samples in the discovery set, the mutant peptide is more likely to bind MHC Class I than the corresponding wild type peptide. FIG. 10H shows across all samples in the validation set, the mutant peptide is more likely to bind MHC Class I than the corresponding wild type peptide. FIGS. 10I and 10J show the frequency of amino acids in common tetrapeptides in LB and NB Groups. The height of each letter reflects the frequency of a given amino acid at that position. FIG. 10K shows the known antigens of which tetrapeptides comprise a substring, arranged by clinical group. Conserved tetrapeptide neoepitopes comprise substrings of antigens from infectious pathogens with evidence in vitro for T cell activation.

FIG. 11 shows polyfunctional CD8 T cell response detected in peptide pools A, B, and C at week 60 blood sample. Frozen PBMCs from patient CR1509, CR9699 and CR9306 were thawed and restimulated with peptide pool A, B, and C, respectively as described in the Methods. Intracellular cytokine staining (ICS) was performed on day 10 with the following conditions: No stimulation (negative control), Staphylococcal enterotoxin B (SEB, positive control) and corresponding peptide pool. Representative dot plots of CD8+IFN-γ+, CD8+IFN-γ+TNF-α+ and CD8+IFN-γ+CD107a+ T cells were shown in FIG. 11A (pool A for patient CR1509), FIG. 11B (pool B for patient CR9699) and FIG. 11C (pool C for patient CR9306). FIG. 11D shows the percent CD8+ IFN-γ, TNF-α, CD-107a and MIP-1β dual positive cells when stimulated with mutant peptide GLEREGFTF as compared to the wild type GLERGGFTF.

FIG. 12 depicts a flowchart of the simulation to test the null hypothesis that a signature would have resulted from a diiferent dataset, either a permutation of the actual data, or a simulated dataset.

FIG. 13 demonstrates that neither mutant nor wild type peptides elicited CD8+ IFN-γ responses in three healthy donors.

FIG. 14 demonstrates that neoantigen generation can be a function of genomic location. Neoantigens from three representative LB patients are plotted as a function of genomic location. Candidate neoepitopes in a signature are generated in different genes. Chromosomal locations of neoepitopes are plotted along the x-axis. Height of peak indicates how many patients share that amino acid sequence in the discovery and validation sets. Tetrapeptides were encoded by mutations in diverse genes across the genome.

FIG. 15 depicts an exome analysis pipeline for a validation set.

FIG. 16 depicts tumor biopsies stained for LCA (leukocyte common antigen), CD8, and FOXP3. According to FIG. 16A, in those with no clinical benefit (NB; A-E) compared to those with long term benefit (LB; F-J) there was no significant difference in the percent of cells staining with LCA (B,G, 200× magnification, arrow tip marks positive cells), CD8 (C,H, 200× magnification, arrow tip marks positive cells), or FOXP3 (D,I, 200× magnification, arrow tip marks positive cells). Tumors from both NB and LB patients show necrosis (E,J, 100× magnification) and the percent of tumor showing necrosis is significantly different (P=0.034) between groups (O), however, this finding is dependent on inclusion of the single outlier value (P=0.683 when excluded). According to FIG. 16B, there is a significant increase (P=0.028) in the CD8:FOXP3 ratio (C) in the LB group compared to the NB. LCA (leukocyte common antigen) appears higher in the LB group but is not statistically significant.

FIG. 17 depicts detailed characteristics of patients in the validation set.

FIG. 18 depicts nonsynonymous exonic mutations per tumor for discovery and validation sets.

FIG. 19 depicts the context, genes and loci for tetrapeptides in a response signature.

FIG. 20 depicts the expression of genes encoding mutations leading to tetrapeptides present in a response signature from a TCGA RNA-seq dataset. After excluding tumors with no expression, the mean SEM value is shown for each gene. If the gene is not expressed in any sample, a zero is shown.

FIG. 21 depicts the sample site, sample size, and type of biopsy for each patient sample.

### DEFINITIONS

In order for the present invention to be more readily understood, certain terms are defined below. Those skilled in the art will appreciate that definitions for certain terms may be provided elsewhere in the specification, and/or will be clear from context.

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

Affinity: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody agent: As used herein, the term “antibody agent” refers to an agent that specifically binds to a particular antigen. In some embodiments, the term encompasses any polypeptide with immunoglobulin structural elements sufficient to confer specific binding. Suitable antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody polypeptide: As used herein, the terms “antibody polypeptide” or “antibody”, or “antigen-binding fragment thereof”, which may be used interchangeably, refer to polypeptide(s) capable of binding to an epitope. In some embodiments, an antibody polypeptide is a full-length antibody, and in some embodiments, is less than full length but includes at least one binding site (comprising at least one, and preferably at least two sequences with structure of antibody “variable regions”). In some embodiments, the term “antibody polypeptide” encompasses any protein having a binding domain which is homologous or largely homologous to an immunoglobulin-binding domain. In particular embodiments, “antibody polypeptides” encompasses polypeptides having a binding domain that shows at least 99% identity with an immunoglobulin binding domain. In some embodiments, “antibody polypeptide” is any protein having a binding domain that shows at least 70%, 80%, 85%, 90%, or 95% identity with an immuglobulin binding domain, for example a reference immunoglobulin binding domain. An included “antibody polypeptide” may have an amino acid sequence identical to that of an antibody that is found in a natural source. Antibody polypeptides in accordance with the present invention may be prepared by any available means including, for example, isolation from a natural source or antibody library, recombinant production in or with a host system, chemical synthesis, etc., or combinations thereof. An antibody polypeptide may be monoclonal or polyclonal. An antibody polypeptide may be a member of any immunoglobulin class, including any of the human classes: IgG, IgM, IgA, IgD, and IgE. In certain embodiments, an antibody may be a member of the IgG immunoglobulin class. As used herein, the terms “antibody polypeptide” or “characteristic portion of an antibody” are used interchangeably and refer to any derivative of an antibody that possesses the ability to bind to an epitope of interest. In certain embodiments, the “antibody polypeptide” is an antibody fragment that retains at least a significant portion of the full-length antibody's specific binding ability. Examples of antibody fragments include, but are not limited to, Fab, Fab′, F(ab′)₂, scFv, Fv, dsFv diabody, and Fd fragments. Alternatively or additionally, an antibody fragment may comprise multiple chains that are linked together, for example, by disulfide linkages. In some embodiments, an antibody polypeptide may be a human antibody. In some embodiments, the antibody polypeptides may be a humanized. Humanized antibody polypeptides include may be chimeric immunoglobulins, immunoglobulin chains or antibody polypeptides (such as Fv, Fab, Fab′, F(ab′)2 or other antigen-binding subsequences of antibodies) that contain minimal sequence derived from non-human immunoglobulin. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In particular embodiments, antibody polyeptides for use in accordance with the present invention bind to particular epitopes of on immune checkpoint molecules.

Antigen: An “antigen” is a molecule or entity to which an antibody binds. In some embodiments, an antigen is or comprises a polypeptide or portion thereof. In some embodiments, an antigen is a portion of an infectious agent that is recognized by antibodies. In some embodiments, an antigen is an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor, and may or may not induce a particular physiological response in an organism. In some embodiments, for example, an antigen may bind to an antibody and/or to a T cell receptor in vitro, whether or not such an interaction occurs in vivo. In general, an antigen may be or include any chemical entity such as, for example, a small molecule, a nucleic acid, a polypeptide, a carbohydrate, a lipid, a polymer [in some embodiments other than a biologic polymer (e.g., other than a nucleic acid or amino acid polymer)] etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is or comprises a recombinant antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

Comparable: The term “comparable” is used herein to describe two (or more) sets of conditions, circumstances, individuals, or populations that are sufficiently similar to one another to permit comparison of results obtained or phenomena observed. In some embodiments, comparable sets of conditions, circumstances, individuals, or populations are characterized by a plurality of substantially identical features and one or a small number of varied features. Those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. Those skilled in the art will appreciate that relative language used herein (e.g., enhanced, activated, reduced, inhibited, etc) will typically refer to comparisons made under comparable conditions.

Consensus sequence: As used herein, the term “consensus sequence” refers to a core sequence that elicits or drives a physiological phenomenon (e.g., an immune response). It is to be understood by those of skill in the art that a a cancer cell that shares a “consensus sequence” with an antigen of an infectious agent shares a portion of amino acid sequence that affects the binding affinity of the antigen to an MHC molecule (either directly or allosterically), and/or facilitates recognition by T cell receptors. In some embodiments, a consensus sequence is a tetrapeptide. In some embodiments, a consensus sequence is a nonapeptide. In some embodiments, a consensus sequence is betwene four and nine amino acids in length. In some embodiments, a consesnsus sequence is greater than nine amino acids in length.

Diagnostic information: As used herein, diagnostic information or information for use in diagnosis is any information that is useful in determining whether a patient has a disease or condition and/or in classifying the disease or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely to have a disease or condition (such as cancer), state, staging or characteristic of the disease or condition as manifested in the subject, information related to the nature or classification of a tumor, information related to prognosis and/or information useful in selecting an appropriate treatment. Selection of treatment may include the choice of a particular therapeutic (e.g., chemotherapeutic) agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, a dosing regimen is or has been correlated with a desired therapeutic outcome, when administered across a population of patients.

Favorable response: As used herein, the term favorable response refers to a reduction of symptoms, full or partial remission, or other improvement in disease pathophysiology. Symptoms are reduced when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. Many cancer patients with smaller tumors have no symptoms. It is not intended that the present invention be limited only to cases where the symptoms are eliminated. The present invention specifically contemplates treatment such that one or more symptoms is/are reduced (and the condition of the subject is thereby “improved”), albeit not completely eliminated. In some embodiments, a favorable response is established when a particular therapeutic regimen shows a statistically significant effect when administered across a relevant population; demonstration of a particular result in a specific individual may not be required. Thus, in some embodiments, a particular therapeutic regimen is determined to have a favorable response when its administration is correlated with a relevant desired effect.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM 120 weight residue table, a gap length penalty of 12 and a gap penalty of 4. The percent identity between two nucleotide sequences can, alternatively, be determined using the GAP program in the GCG software package using an NWSgapdna.CMP matrix.

Immune checkpoint modulator: As used herein, the term “immune checkpoint modulator” refers to an agent that interacts directly or indirectly with an immune checkpoint. In some embodiments, an immune checkpoint modulator increases an immune effector response (e.g., cytotoxic T cell response), for example by stimulating a positive signal for T cell activation. In some embodiments, an immune checkpoint modulator increases an immune effector response (e.g., cytotoxic T cell response), for example by inhibiting a negative signal for T cell activation (e.g. disinhibition). In some embodiments, an immune checkpoint modulator interferes with a signal for T cell anergy. In some embodiments, an immune checkpoint modulator reduces, removes, or prevents immune tolerance to one or more antigens.

Long Term Benefit: In general, the term “long term benefit” refers to a desirable clinical outcome, e.g., observed after administration of a particular treatment or therapy of interest, that is maintained for a clinically relevant period of time. To give but one example, in some embodiments, a long term benefit of cancer therapy is or comprises (1) no evidence of disease (“NED”, for example upon radiographic assessment) and/or (2) stable or decreased volume of diseases. In some embodiments, a clinically relevant period of time is at least 1 month, at least 2 months, at least 3 months, at least 4 months, at least 5 months or more. In some embodiments, a clinically relevant period of time is at least six months. In some embodiments, a clinically relevant period of time is at least 1 year.

Marker: A marker, as used herein, refers to an agent whose presence or level is a characteristic of a particular tumor or metastatic disease thereof. For example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

Modulator: The term “modulator” is used to refer to an entity whose presence in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

Neoepitope: A “neoepitope” is understood in the art to refer to an epitope that emerges or develops in a subject after exposure to or occurrence of a particular event (e.g., development or progression of a particular disease, disorder or condition, e.g., infection, cancer, stage of cancer, etc). As used herein, a neoepitope is one whose presence and/or level is correlated with exposure to or occurrence of the event. In some embodiments, a neoepitope is one that triggers an immune response against cells that express it (e.g., at a relevant level). In some embodiments, a neopepitope is one that triggers an immune response that kills or otherwise destroys cells that express it (e.g., at a relevant level). In some embodiments, a relevant event that triggers a neoepitope is or comprises somatic mutation in a cell. In some embodiments, a neoepitope is not expressed in non-cancer cells to a level and/or in a manner that triggers and/or supports an immune response (e.g., an immune response sufficient to target cancer cells expressing the neoepitope).

No Benefit: As used herein, the phrase “no benefit” is used to refer to absence of detectable clinical benefit (e.g., in response to administration of a particular therapy or treatment of interest). In some embodiments, absence of clinical benefit refers to absence of statistically significant change in any particular symptom or characteristic of a particular disease, disorder, or condition. In some embodiments, absence of clinical benefit refers to a change in ore or more symptoms or characteristics of a disease, disorder, or condition, that lasts for only a short period of time such as, for example, less than about 6 months, less than about 5 months, less than about 4 months, less than about 3 months, less than about 2 months, less than about 1 month, or less.

Patient: As used herein, the term “patient” or “subject” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the disorder or condition is metastatic cancer.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Prognostic and predictive information: As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Reference sample: As used herein, a reference sample may include, but is not limited to, any or all of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification or reverse transcription of mRNA, etc.

Response: As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et. al., “New guidelines to evaluate the response to treatment in solid tumors”, European Organization for Research and Treatment of Cancer, National Cancer Institute of the United States, National Cancer Institute of Canada, J. Natl. Cancer Inst., 2000, 92(3):205-216. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Sample: As used herein, a sample obtained from a subject may include, but is not limited to, any or all of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification or reverse transcription of mRNA, etc.

Specific binding: As used herein, the terms “specific binding” or “specific for” or “specific to” refer to an interaction (typically non-covalent) between a target entity (e.g., a target protein or polypeptide) and a binding agent (e.g., an antibody, such as a provided antibody). As will be understood by those of ordinary skill, an interaction is considered to be “specific” if it is favored in the presence of alternative interactions. In many embodiments, an interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. In particular embodiments, an antibody specific for receptor tyrosine kinases has less than 10% cross-reactivity with receptor tyrosine kinase bound to protease inhibitors (e.g., ACT). One of ordinary skill in the art will be able to select antibodies having a sufficient degree of specificity to perform appropriately in any given application (e.g., for detection of a target molecule, for therapeutic purposes, etc.). Specificity may be evaluated in the context of additional factors such as the affinity of the binding molecule for the target molecule versus the affinity of the binding molecule for other targets (e.g., competitors). If a binding molecule exhibits a high affinity for a target molecule that it is desired to detect and low affinity for non-

[symbol]

Stage of cancer: As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

Subject: As used herein, the term “subject” or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition (e.g., a cancer) has been diagnosed with and/or exhibits one or more symptoms of the disease, disorder, or condition. In some embodiments, an individual who is suffering from cancer has cancer, but does not display any symptoms of cancer and/or has not been diagnosed with a cancer.

Susceptible to: An individual who is “susceptible to” a disease, disorder, or condition (e.g., cancer) is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who displays conditions associated with development of the disease, disorder, or condition. In some embodiments, a risk of developing a disease, disorder, and/or condition is a population-based risk.

Target cell or target tissue: As used herein, the terms “target cell” or “target tissue” refer to any cell, tissue, or organism that is affected by a condition described herein and to be treated, or any cell, tissue, or organism in which a protein involved in a condition described herein is expressed. In some embodiments, target cells, target tissues, or target organisms include those cells, tissues, or organisms in which there is a detectable amount of immune checkpoint signaling and/or activity. In some embodiments, target cells, target tissues, or target organisms include those cells, tissues or organisms that display a disease-associated pathology, symptom, or feature.

Therapeutic regimen: As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include a treatment or series of treatments designed to achieve a particular effect, e.g., reduction or elimination of a detrimental condition or disease such as cancer. The treatment may include administration of one or more compounds either simultaneously, sequentially or at different times, for the same or different amounts of time. Alternatively, or additionally, the treatment may include exposure to radiation, chemotherapeutic agents, hormone therapy, or surgery. In addition, a “treatment regimen” may include genetic methods such as gene therapy, gene ablation or other methods known to reduce expression of a particular gene or translation of a gene-derived mRNA.

Therapeutic agent: As used herein, the phrase “therapeutic agent” refers to any agent that, when administered to a subject, has a therapeutic effect and/or elicits a desired biological and/or pharmacological effect.

Therapeutically effective amount: As used herein, the term “therapeutically effective amount” refers to an amount of an agent (e.g., an immune checkpoint modulator) that confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic agent or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific fusion protein employed; the duration of the treatment; and like factors as is well known in the medical arts.

Treatment: As used herein, the term “treatment” (also “treat” or “treating”) refers to any administration of a substance (e.g., provided compositions) that partially or completely alleviates, ameliorates, relieves, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

### DETAILED DESCRIPTION OF CERTAIN EMBODIMENTS

The present invention encompasses the discovery that a high mutational load and somatic neoepitopes formed as a result of tumor mutations contribute to the anti-tumor immune response of immune checkpoint modulators.

Among other things, the present disclosure specifically demonstrates that neoepitopes in cancer cells are associated with increased binding affinity to MHC class I molecules and/or improved recognition by cytotoxic T cells.

The present invention provides, among other things, methods for detecting somatic neoepitopes present in cancer cells and/or establishing association between or among such neoepitopes and responsiveness to immunitherapy. In some emodiments, the present invention provides methods and/or reagents for identifying cancer patients that are likely to respond favorably to treatment with immunotherapy (e.g., with an immune checkpoint modulator) and/or for selecting patients to receive such immunotherapy. Alternatively or additionally, the present invention provides methods and/or reagents for treating patients with an immune checkpoint modulator that have been identified to have cancer harboring a somatic neoepitope.

### Somatic Mutations

Somatic mutations comprise DNA alterations in non-germline cells and commonly occur in cancer cells. It has been discovered herein that certain somatic mutations in cancer cells result in the expression of neoepitopes, that in some embodiments transition a stretch of amino acids from being recognized as “self” to “non-self”. According to the present invention, a cancer cell harboring a “non-self” antigen is likely to elicit an immune response against the cancer cell. Immune responses against cancer cells can be enhanced by an immune checkpoint modulator. The present invention teaches that cancers expressing neoepitopes may be more responsive to therapy with immune checkpoint modulator. Among other things, the present invention provides strategies for improving cancer therapy by permitting identification and/or selection of particular patients to receive (or avoid) therapy. The present invention also provides technologies for defining neoeptiopes, or sets thereof, whose presence is indicative of a particular clinical outcome of interest (e.g., responsiveness to therapy, for example with a particular immune checkpoint modulator and/or risk of developing a particular undesirable side effect of therapy). The present invention defines and/or permits definition of one or more neoepitope “signatures” associated with beneficial (or undesirable) response to immune checkpoint modulator therapy.

In some embodiments, a somatic mutation results in a neoantigen or neoepitope. Among other things, the present disclosure demonstrates the existence of neoepitopes, arising from somatic mutation, whose presence is associated with a particular response to immune checkpoint modulator therapy. In some embodiments, a neoepitope is or comprises a tetrapeptide, for example that contributes to increased binding affinity to MHC Class I molecules and/or recognition by cells of the immune system (i.e. T cells) as “non-self”. In some particular embodiments, a somatic mutation results in a neoepitope comprising a tetrapeptide listed in Table 1. In some embodiments, a neoepitope shares a consensus sequence with an antigen from an infectious agent.

In some embodiments, a neoepitope signature of interest in accordance with the present invention is or comprises a neoepitope or set thereof whose presence in a tumor sample correlates with a particular clinical outcome. The present disclosure demonstrates the effective definition of such a neoepitope signature. In some embodiments, a useful signature is or comprises one or more of the consensus tetrapeptide somatic neoeptopes found in Table 1; in some embodiments, a useful signature is or comprises one or more of the tetrapeptide somatic neoepitopes underlined in Table 2; in some embodiments, a useful signature is or comprises one or more of the nonamer peptides found in Table 2. In some embodiments, a useful signature is or comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 7-, 71, 72, 73, 74, 75, or more neoepitopes. In some embodiments, the present disclosure provides technologies for defining and/or detecting neopetiope signatures, and particulary those relevant to immune checkpoint modulator therapy.

Among other things, the present disclosure demonstrates definition of neoepitopes and neoepitope signatures associated with a particular response or response feature (e.g., responsiveness to therapy or risk of side effect) of immune checkpoint modulator therapy. In the particular Examples presented herein, such definition is achieved by comparing genetic sequence information from a first plurality of tumor samples, which first plurality contains samples that share a common response feature to immune checkpoint modulator therapy, with that obtained from a second plurality of tumor samples, which second plurality contains samples that do not share the common response feature but are otherwise comparable to those of the first set, so that the comparison defines genetic sequence elements whose presence is associated or correlates with the common response feature. The present disclosure specifically demonstrates that increased mutational burden can correlate with a response feature (e.g., with responsiveness to therapy), but also demonstrates that such increased mutational burden alone may not be sufficient to predict the response feature. The present disclosure demonstrates that, when such somatic mutation generates neoeptiopes, a useful neoeptiope signature associated with the response feature can be defined. The present disclosure provides specific technologies for defining and utilizing such signatures.

In some embodiments, a cancer cell comprising a neoepitope is selected from a carcinoma, sarcoma, melanoma, myeloma, leukemia, or lymphoma. In some embodiments, a cancer cell comprising a neoepitope is a melanoma. In some embodiments, a cancer cell comprising a neoepitope is a non-small-cell lung carcinoma.

TABLE 1
Exemplary consensus tetrapeptide somatic
neoepitopes in melanoma

| | SEQ |
|---|---|
| | ID |
| Tetramer | NO. |
| AARA | 1 |
| ALLN | 2 |
| ALSV | 3 |
| AVLS | 4 |
| DSSE | 5 |
| EADL | 6 |
| KEEF | 7 |
| LERE | 8 |
| LSLA | 9 |
| LSSV | 10 |
| PNSS | 11 |
| SLGL | 12 |
| SSGL | 13 |
| SSVL | 14 |
| EKLS | 15 |
| FLGS | 16 |
| FSLN | 17 |
| KKIL | 18 |
| LSLL | 19 |
| LTAT | 20 |
| QLPP | 21 |
| SASA | 22 |
| SSAF | 23 |
| VLSS | 24 |
| DKSL | 25 |
| EVLL | 26 |
| LAPE | 27 |
| LKEL | 28 |
| LLFL | 29 |
| LLQL | 30 |
| LPPL | 31 |
| LSPG | 32 |
| PPLL | 33 |
| RGSS | 34 |
| SPPP | 35 |
| SPSS | 36 |
| SSLE | 37 |
| SSRS | 38 |
| VAAL | 39 |
| EEEE | 40 |
| LAAL | 41 |
| LGSL | 42 |
| LKKK | 43 |
| LLLL | 44 |
| LLLV | 45 |
| LLSL | 46 |
| LPPP | 47 |
| LSSL | 48 |
| SSLA | 49 |
| VTKE | 50 |
| ELEE | 51 |
| KIKA | 52 |
| KILS | 53 |
| KLGI | 54 |
| KLPA | 55 |
| LSKA | 56 |
| PPSQ | 57 |
| QKLG | 58 |
| SLLA | 59 |
| VSFV | 60 |
| EDLL | 61 |
| EILE | 62 |
| LENF | 63 |
| VLEE | 64 |
| GPSP | 65 |
| GSFS | 66 |
| LFGN | 67 |
| LKKR | 68 |
| PFLP | 69 |
| PPPP | 70 |
| RKLS | 71 |
| LSLS | 72 |
| LLKK | 126 |
| ESSA | 127 |

TABLE 2
Neoepitope Sets Associated with Response to
CTLA-4 Blockade (e.g., via Ipilimumab
Treatment).
Tetrapeptide neoepitopes in each nonamer
are underlined.

| CR signature | | | | CR + long SD signature | | | |
|---|---|---|---|---|---|---|---|
| | SEQ | | SEQ | | SEQ | | SEQ |
| Tetra- | ID | Mutant | ID | Tetra- | ID | Mutant | ID |
| peptide | NO | 9mer | NO | peptide | NO | 9mer | NO |
| AARA | 1 | RTAARAVSP | 73 | EKLS | 15 | REKLSILCT | 126 |
| | | QGAARARVL | 74 | | | QEKLSIRQG | 127 |
| | | EEAARAVDD | 75 | | | KPNNEKLST | 128 |
| ALLN | 2 | RLVALLNHI | 76 | | | QEQEEKLSF | 129 |
| | | SLSALLNIF | 77 | | | RYTTIEKLS | 130 |
| | | ALLNLSSRC | 78 | FLGS | 16 | FLGSLGAEG | 131 |
| ALSV | 3 | VPALSVITD | 79 | | | GSSDFLGSG | 132 |
| | | ALSVSGKRE | 80 | | | GNVVFLGSA | 133 |
| | | SQQYQALSV | 81 | | | SEKTCFLGS | 134 |
| AVLS | 4 | LAVLSSLFL | 82 | | | NSCILFLGS | 135 |
| | | SRAVLSSFS | 83 | | | LPPDNFLGS | 136 |
| | | NTSAVLSQS | 84 | FSLN | 17 | VSILFSLNL | 137 |
| | | AVLSLPGAQ | 85 | | | VFSLNPDTG | 138 |
| DSSE | 5 | GDSSEDSSG | 86 | | | KFSLNGGYW | 139 |
| | | DSSEIGAVL | 87 | | | GWANFSLNP | 140 |
| | | ALGDSSERV | 88 | | | QFSLNRGCK | 141 |
| EADL | 6 | AEILEADLQ | 89 | KKIL | 18 | SLKAIKKIL | 142 |
| | | DAEADLVGR | 90 | | | VHGKKILRT | 143 |
| | | VEADLTAVG | 91 | | | VKSMKKKIL | 144 |
| KEEF | 7 | NIAVKEEFN | 92 | | | SATKKILIV | 145 |
| | | IKEEFDYIS | 93 | | | LKRKKKILS | 146 |
| | | QGEEIKEEF | 94 | LSLL | 19 | LLSLLVTTS | 147 |
| LERE | 8 | EEDALEREG | 95 | | | HKVLSLLWN | 148 |
| | | GLEREGFTF | 96 | | | IGRLSLLNP | 149 |
| | | REIVXLERE | 97 | | | SFLSLLFFC | 150 |
| LSLA | 9 | KRLLSLATT | 98 | LTAT | 20 | KGETLTATP | 151 |
| | | ISYLSLAHM | 99 | | | AHNLCLTAT | 152 |
| | | GDVMFLSLA | 100 | | | VPDSLTATT | 153 |
| | | LFNDHLSLA | 101 | | | NLTATEVVV | 154 |
| LSSV | 10 | LSSVFFVEV | 102 | QLPP | 21 | KSPSNQLPP | 155 |
| | | ISPLLSSVL | 103 | | | KSPSNQLPP | 156 |
| | | LLSSVDGVS | 104 | | | SVGDCQLPP | 157 |
| PNSS | 11 | CNPNSSGLN | 105 | | | FLSQNQLPP | 158 |
| | | FMYLQPNSS | 106 | SASA | 22 | SASATHQAD | 159 |
| | | PVGPNSSKG | 107 | | | VCSASAGRN | 160 |
| SLGL | 12 | FLDSSLGLC | 108 | | | YMDLMSASA | 161 |
| | | KLSSLGLRG | 109 | | | SSKGLSASA | 162 |
| | | GPASLGLPA | 110 | SSAF | 23 | GTVSSSAFL | 163 |
| SSGL | 13 | CNPNSSGLN | 111 | | | YPFSSSAFN | 164 |
| | | PGLFSSGLY | 112 | | | ESSAFLLNS | 165 |
| | | GPASSGLPA | 113 | | | LSSAFRRSC | 166 |
| | | EFRGSSGLL | 114 | VLSS | 24 | DYVLSSEYY | 167 |
| SSLA | 49 | FSTNSSLAK | 115 | | | LAVLSSLFL | 168 |
| | | QGMPSSLAQ | 116 | | | SRAVLSSFS | 169 |
| | | SVLPSSLAA | 117 | | | VLSSLEGNI | 170 |
| SSLE | 37 | EDILNSSLE | 118 | | | AVLSSPGAQ | 171 |
| | | SGSSLEKEL | 119 | | | VMQGIVLSS | 172 |
| | | KQKSSLETP | 120 | | | | |
| | | VLSSLEGNI | 121 | | | | |
| | | YTTSSLECG | 122 | | | | |
| SSVL | 14 | ISPLLSSVL | 123 | | | | |
| | | SPSSVLGFH | 124 | | | | |
| | | SSVLPVNGK | 125 | | | | |

### Immune Checkpoint Modulation

Immune checkpoints refer to inhibitory pathways of the immune system that are responsible for maintaining self-tolerance and modulating the duration and amplitude of physiological immune responses.

Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, immune checkpoint modulators may be administered to overcome the inhibitory signals and permit and/or augment an immune attack against cancer cells. Immune checkpoint modulators may facilitate immune cell responses against cancer cells by decreasing, inhibiting, or abrogating signaling by negative immune response regulators (e.g. CTLA4), or may stimulate or enhance signaling of positive regulators of immune response (e.g. CD28).

Immunotherapy agents targeted to immune checkpoint modulators may be administered to encourage immune attack targeting cancer cells. Immunotherapy agents may be or include antibody agents that target (e.g., are specific specific for) immune checkpoint modulators. Examples of immunotherapy agents include antibody agents targeting one or more of CTLA-4, PD-1, PD-L1, GITR, OX40, LAG-3, KIR, TIM-3, CD28, CD40; and CD137.

Specific examples of antibody agents may include monoclonal antibodies. Certain monoclonal antibodies targeting immune checkpoint modulators are available. For instance, ipilumimab targets CTLA-4; tremelimumab targets CTLA-4; pembrolizumab targets PD-1, etc.

### Detection of Neoepitopes

Cancers may be screened to detect neoepitopes using any of a variety of known technologies. In some embodiments, neoepitopes, or expression thereof, is detected at the nucleic acid level (e.g., in DNA or RNA). In some embodiments, neopeitopes, or expression thereof, is detected at the protein level (e.g., in a sample comprising polypeptides from cancer cells, which sample may be or comprise polypeptide complexes or other higher order structures including but not limited to cells, tissues, or organs).

In some particular embodiments, one or more neoepitopes are detected by whole exome sequencing. In some embodiments, one or more neoepitopes are detected by immunoassay. In some embodiments, one or more neoepitopes are detected by microarray. In some embodiments, one or more neoepitopes may be detected using massively parallel exome sequencing sequencing. In some embodiments, one or more neoepitopes may be detected by genome sequencing. In some embodiments, one or more neoepitopes may be detected by RNA sequencing. In some embodiments, one or more neoepitopes may be detected by standard DNA or RNA sequencing. In some embodiments, one or more neoepitopes may be detected by mass spectrometry.

In some embodiments, one or more neoepitopes may be detected at the nucleic acid level using next generation sequencing (DNA and/or RNA). In some embodiments, Next-neoepitopes, or expression thereof may be detected using genome sequencing, genome resequencing, targeted sequencing panels, transcriptome profiling (RNA-Seq), DNA-protein interactions (ChIP-sequencing), and/or epigenome characterization. In some embodiments, re-sequencing of a patient's genome may be utilized, for example to detect genomic variations.

In some embodiments, one or more neoepitopes may be detected using a technique such as ELISA, Western Tranfer, immunoassay, mass spectrometry, microarray analysis, etc.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### Methods of Treatment

In some embodiments, the invention provides methods for identifying cancer patients that are likely to respond favorably to treatment with an immune checkpoint modulator. In some embodiments, the invention provides methods for identifying a cancer patient that is likely to respond favorably to treatment with an immune checkpoint modulator and treating the patient with an immune checkpoint modulator. In some embodiments, the invention provides methods of treating a cancer patient with an immune checkpoint modulator who has previously been identified as likely to respond favorably to treatment with an immune checkpoint modulator. In some embodiments, the invention provides methods for identifying a cancer patient that is not likely to respond favorably to treatment with an immune checkpoint modulator and not treating the patient with an immune checkpoint modulator. In some embodiments, the invention provides methods for identifying a cancer patient who is likely to suffer one or more autoimmune complications if administered an immune checkpoint modulator. In some embodiments, the invention provides methods for treating a cancer patient with an immunosuppressant who has previously identified as likely to suffer one or more autoimmune complications if treated with an immune checkpoint modulator. In some embodiments, the immunosuppressant is administered to the patient prior to or concomitantly with an immune checkpoint modulator.

Administration of Immune Checkpoint Modulators

In accordance with certain methods of the invention, an immune checkpoint modulator is or has been administered to an individual. In some embodiments, treatment with an immune checkpoint modulator is utilized as a sole therapy. In some embodiments, treatement with an immune checkpoint modulator is used in combination with one or more other therapies.

Those of ordinary skill in the art will appreciate that appropriate formulations, indications, and dosing regimens are typically analyzed and approved by government regulatory authorities such as the Food and Drug Administration in the United States. For example, Example 5 presents certain approved dosing information for ipilumimab, an anti-CTL-4 antibody. In many embodiments, an immune checkpoint modulator is administered in accordance with the present invention according to such an approved protocol. However, the present disclosure provides certain technologies for identifying, characterizing, and/or selecting particular patients to whom immune checkpoint modulators may desirably be administered. In some embodiments, insights provided by the present disclosure permit dosing of a given immune checkpoint modulator with greater frequency and/or greater individual doses (e.g., due to reduced susceptibiloity to and/or incidence or intensity of undesirable effects) relative to that recommended or approved based on population studies that include both individuals identified as described herein (e.g., expressing neoepitopes) and other individuals. In some embodiments, insights provided by the present disclosure permit dosing of a given immune checkpoint modulator with reduced frequency and/or reduced individual doses (e.g., due to increased responsiveness) relative to that recommended or approved based on population studies that include both individuals identified as described herein (e.g., expressing neoepitopes) and other individuals.

In some embodiments, an immune system modulator is administered in a pharmaceutical composition that also comprises a physiologically acceptable carrier or excipient. In some embodiments, a pharmaceutical composition is sterile. In many embodiments, a pharmaceutical composition is formulated for a particular mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrrolidone, etc., as well as combinations thereof. A pharmaceutical preparation can, if desired, comprise one or more auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In some embodiments, a water-soluble carrier suitable for intravenous administration is used.

In some embodiments, a pharmaceutical composition or medicament, if desired, can contain an amount (typically a minor amount) of wetting or emulsifying agents, and/or of pH buffering agents. In some embodiments, a pharmaceutical composition can be a liquid solution, suspension, emulsion, tablet, pill, capsule, sustained release formulation, or powder. In some embodiments, a pharmaceutical composition canbe formulated as a suppository, with traditional binders and carriers such as triglycerides. Oral formulation can include standard carriers such as pharmaceutical grades of mannitol, lactose, starch, magnesium stearate, polyvinyl pyrrolidone, sodium saccharine, cellulose, magnesium carbonate, etc.

In some embodiments, a pharmaceutical composition can be formulated in accordance with the routine procedures as a pharmaceutical composition adapted for administration to human beings. For example, in some embodiments, a composition for intravenous administration typically is a solution in sterile isotonic aqueous buffer. Where necessary, acomposition may also include a solubilizing agent and a local anesthetic to ease pain at the site of the injection. Generally, ingredients are supplied either separately or mixed together in unit dosage form, for example, as a dry lyophilized powder or water free concentrate in a hermetically sealed container such as an ampule or sachet indicating the quantity of active agent. Where a composition is to be administered by infusion, it can be dispensed with an infusion bottle containing sterile pharmaceutical grade water, saline or dextrose/water. Where a composition is administered by injection, an ampule of sterile water for injection or saline can be provided so that the ingredients may be mixed prior to administration.

In some embodiments, an immune checkpoint modulator can be formulated in a neutral form; in some embodiments it may be formulated in a salt form. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Pharmaceutical compositions for use in accordance with the present invention may be administered by any appropriate route. In some embodiments, a pharmaceutical compostion is administered intravenously. In some embodiments, a pharmaceutical composition is administered subcutaneously. In some embodiments, a pharmaceutical composition is administered by direct administration to a target tissue, such as heart or muscle (e.g., intramuscular), or nervous system (e.g., direct injection into the brain; intraventricularly; intrathecally). Alternatively or additionally, in some embodiments, a pharmaceutical composition is administered parenterally, transdermally, or transmucosally (e.g., orally or nasally). More than one route can be used concurrently, if desired.

Immune checkpoint modulators (or a composition or medicament containing an immune checkpoint modulator, can be administered alone, or in conjunction with other immune checkpoint modulators. The term, “in conjunction with,” indicates that a first immune checkpoint modulator is administered prior to, at about the same time as, or following another immune checkpoint modulator. For example, a first immune checkpoint modulator can be mixed into a composition containing one or more different immune checkpoint modulators, and thereby administered contemporaneously; alternatively, the agent can be administered contemporaneously, without mixing (e.g., by “piggybacking” delivery of the agent on the intravenous line by which the immune checkpoint modulator is also administered, or vice versa). In another example, the immune checkpoint modulator can be administered separately (e.g., not admixed), but within a short time frame (e.g., within 24 hours) of administration of the immune checkpoint modulator.

In some embodiments, subjects treated with immune checkpoint modulators are administered one or more immunosuppressants. In some embodiments, one or more immunosuppressants are administered to decrease, inhibit, or prevent an undesired autoimmune response (e.g., enterocolitis, hepatitis, dermatitis (including toxic epidermal necrolysis), neuropathy, and/or endocrinopathy), for example, hypothyroidism. Exemplary immunosuppressants include steroids, antibodies, immunoglobulin fusion proteins, and the like. In some embodiments, an immunosuppressant inhibits B cell activity (e.g. rituximab). In some embodiments, an immunosuppressant is a decoy polypeptide antigen.

In some embodiments, immune checkpoint modulators (or a composition or medicament containing immune checkpoint modulators) are administered in a therapeutically effective amount (e.g., a dosage amount and/or according to a dosage regimen that has been shown, when administered to a relevant population, to be sufficient to treat cancer, such as by ameliorating symptoms associated with the cancer, preventing or delaying the onset of the cancer, and/or also lessening the severity or frequency of symptoms of cancer). In some embodiments, long term clinical benefit is observed after treatment with immune checkpoint modulators, including, for example, CTLA-4 blockers such as ipilumimab or tremelimumab, and/or other agents. Those of ordinary skill in the art will appreciate that a dose which will be therapeutically effective for the treatment of cancer in a given patient may depend, at least to some extent, on the nature and extent of cancer, and can be determined by standard clinical techniques. In some embodiments, one or more in vitro or in vivo assays may optionally be employed to help identify optimal dosage ranges. In some embodmients, a particular dose to be employed in the treatment of a given individual may depend on the route of administration, the extent of cancer, and/or one or more other factors deemed relevant in the judgment of a practitioner in light of patient's circumstances. In some embodiments, effective doses may be extrapolated from dose-response curves derived from in vitro or animal model test systems (e.g., as described by the U.S. Department of Health and Human Services, Food and Drug Administration, and Center for Drug Evaluation and Research in “Guidance for Industry: Estimating Maximum Safe Starting Dose in Initial Clinical Trials for Therapeutics in Adult Healthy Volunteers”, Pharmacology and Toxicology, July 2005.

In some embodiments, a therapeutically effective amount of an immune check point modulator can be, for example, more than about 0.01 mg/kg, more than about 0.05 mg/kg, more than about 0.1 mg/kg, more than about 0.5 mg/kg, more than about 1.0 mg/kg, more than about 1.5 mg/kg, more than about 2.0 mg/kg, more than about 2.5 mg/kg, more than about 5.0 mg/kg, more than about 7.5 mg/kg, more than about 10 mg/kg, more than about 12.5 mg/kg, more than about 15 mg/kg, more than about 17.5 mg/kg, more than about 20 mg/kg, more than about 22.5 mg/kg, or more than about 25 mg/kg body weight. In some embodiments, a therapeutically effective amount can be about 0.01-25 mg/kg, about 0.01-20 mg/kg, about 0.01-15 mg/kg, about 0.01-10 mg/kg, about 0.01-7.5 mg/kg, about 0.01-5 mg/kg, about 0.01-4 mg/kg, about 0.01-3 mg/kg, about 0.01-2 mg/kg, about 0.01-1.5 mg/kg, about 0.01-1.0 mg/kg, about 0.01-0.5 mg/kg, about 0.01-0.1 mg/kg, about 1-20 mg/kg, about 4-20 mg/kg, about 5-15 mg/kg, about 5-10 mg/kg body weight. In some embodiments, a therapeutically effective amount is about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg about 1.4 mg/kg, about 1.5 mg/kg, about 1.6 mg/kg, about 1.7 mg/kg, about 1.8 mg/kg, about 1.9 mg/kg, about 2.0 mg/kg, about 2.5 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, body weight, or more. In some embodiments, the therapeutically effective amount is no greater than about 30 mg/kg, no greater than about 20 mg/kg, no greater than about 15 mg/kg, no greater than about 10 mg/kg, no greater than about 7.5 mg/kg, no greater than about 5 mg/kg, no greater than about 4 mg/kg, no greater than about 3 mg/kg, no greater than about 2 mg/kg, or no greater than about 1 mg/kg body weight or less.

In some embodiments, the administered dose for a particular individual is varied (e.g., increased or decreased) over time, depending on the needs of the individual.

In yet another example, a loading dose (e.g., an initial higher dose) of a therapeutic composition may be given at the beginning of a course of treatment, followed by administration of a decreased maintenance dose (e.g., a subsequent lower dose) of the therapeutic composition.

Without wishing to be bound by any theories, it is contemplated that a loading dose may clear out an initial and, in some cases massive, accumulation of undesirable materials (e.g., fatty materials and/or tumor cells, etc) in tissues (e.g., in the liver), and maintenance dosing may delay, reduce, or prevent buildup of fatty materials after initial clearance.

It will be appreciated that a loading dose and maintenance dose amounts, intervals, and duration of treatment may be determined by any available method, such as those exemplified herein and those known in the art. In some embodiments, a loading dose amount is about 0.01-1 mg/kg, about 0.01-5 mg/kg, about 0.01-10 mg/kg, about 0.1-10 mg/kg, about 0.1-20 mg/kg, about 0.1-25 mg/kg, about 0.1-30 mg/kg, about 0.1-5 mg/kg, about 0.1-2 mg/kg, about 0.1-1 mg/kg, or about 0.1-0.5 mg/kg body weight. In some embodiments, a maintenance dose amount is about 0-10 mg/kg, about 0-5 mg/kg, about 0-2 mg/kg, about 0-1 mg/kg, about 0-0.5 mg/kg, about 0-0.4 mg/kg, about 0-0.3 mg/kg, about 0-0.2 mg/kg, about 0-0.1 mg/kg body weight. In some embodiments, a loading dose is administered to an individual at regular intervals for a given period of time (e.g., 1, 2, 3, 4, 5, 6, 7, 8, 9, 10, 11, 12 or more months) and/or a given number of doses (e.g., 1, 2, 3, 4, 5, 6, 7, 8, 9, 10, 15, 20, 25, 30 or more doses), followed by maintenance dosing. In some embodiments, a maintenance dose ranges from 0-2 mg/kg, about 0-1.5 mg/kg, about 0-1.0 mg/kg, about 0-0.75 mg/kg, about 0-0.5 mg/kg, about 0-0.4 mg/kg, about 0-0.3 mg/kg, about 0-0.2 mg/kg, or about 0-0.1 mg/kg body weight. In some embodiments, a maintenance dose is about 0.01, 0.02, 0.04, 0.06, 0.08, 0.1, 0.2, 0.3, 0.4, 0.5, 0.6, 0.7, 0.8, 0.9, 1.0, 1.2, 1.4, 1.6, 1.8, or 2.0 mg/kg body weight. In some embodiments, maintenance dosing is administered for 1, 2, 3, 4, 5, 6, 7, 8, 9, 10, 11, 12 or more months. In some embodiments, maintenance dosing is administered for 1, 2, 3, 4, 5, 6, 7, 8, 9, 10 or more years. In some embodiments, maintenance dosing is administered indefinitely (e.g., for life time).

A therapeutically effective amount of an immune checkpoint modulator may be administered as a one-time dose or administered at intervals, depending on the nature and extent of the cancer, and on an ongoing basis. Administration at an “interval,” as used herein indicates that the therapeutically effective amount is administered periodically (as distinguished from a one-time dose). The interval can be determined by standard clinical techniques. In some embodiments, an immune checkpoint modulator is administered bimonthly, monthly, twice monthly, triweekly, biweekly, weekly, twice weekly, thrice weekly, or daily. The administration interval for a single individual need not be a fixed interval, but can be varied over time, depending on the needs and rate of recovery of the individual.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

The invention additionally pertains to a pharmaceutical composition comprising an immune checkpoint modulator, as described herein, in a container (e.g., a vial, bottle, bag for intravenous administration, syringe, etc.) with a label containing instructions for administration of the composition for treatment of cancer.

### EXAMPLES

The following examples are provided so as to describe to those of ordinary skill in the art how to make and use methods and compositions of the invention, and are not intended to limit the scope of what the inventors regard as their invention.

### Overview

Immune checkpoint blockade is a new therapeutic paradigm that has led to durable anti-tumor effects in patients with metastatic melanoma, non-small cell lung cancer, and other tumor types, but what determines whether a patient will respond remains elusive.¹⁻⁵ This is one of the most critical unanswered questions in the field of cancer immunotherapy. The fully human monoclonal antibodies ipilimumab and tremelimumab block cytotoxic T-lymphocyte antigen 4 (CTLA-4), resulting in T cell activation.^{4,6} Pembrolizumab is drug that targets the programmed cell death 1 (PD-1) receptor as a treatment for metastatic melanoma. A number of studies have established correlations between outcomes to ipilimumab and peripheral blood lymphocyte count, antigen specific immunity, markers of T cell activation,^{7,8} an “inflammatory” microenvironment⁹⁻¹², and maintenance of high-frequency TCR clonotypes.⁶⁹

It is unknown, however, whether a tumor's genetic profile dictates response to CTLA-4 blockade (e.g., via ipilimumab). Relationships between and among tumor genetic landscape, mutation load, and benefit from treatment have been the subject of investigation. Immunogenicity resulting from nonsynonymous melanoma mutations has been illustrated in a mouse model,¹³ and the antigenic diversity of human melanoma tumors has been modeled in silico.¹⁴ Effector and helper T cell function and regulatory T-cell depletion are necessary for anti-CTLA-4 efficacy,¹⁵⁻¹⁷ as is depletion of regulatory T cells¹⁸ but no association between specific HLA type and clinical benefit has been observed.²⁶ Melanomas have the greatest mutational burden (0.5 to greater than 100 mutations per megabase) of any solid tumor.¹⁹⁻²⁰ Studies have shown that somatic mutations can give rise to neoepitopes^{21,22} and that these may serve as neoantigens in preclinical models and in patients.²³⁻²⁵ The hypothesis that ipilumimab response is dictated by the tumor cell genome is relevant. Previous research has demonstrated a lack of association between specific HLA type and ipilimumab response.²⁶ This study investigates whether a tumor's genetic landscape determines clinical response to CTLA-4 blockade (e.g., via treatement with agents such as ipilimumab or tremelimumab).¹⁸

To explore this hypothesis, for a discovery set, we conducted whole exome sequencing of DNA from tumor and matched normal blood of 25 ipilimumab-treated patients (Table 3), followed by an additional 39 tumors as validation, of whom five were treated with tremelimumab. We found that a higher mutational burden was correlated with, but alone was insufficient to predict, a strong clinical benefit from CTLA-4 blockade (e.g. via ipilimumab or tremelimumab). Instead, mutations in tumors from patients with clinical benefit from CTLA-4 blockade harbored shared somatic neoepitopes. Here, we demonstrate a genetic basis for clinical response to immune checkpoint inhibition and define a neoepitope landscape underlying response to therapy.

Those skilled in the art, reading the present disclosure will appreciate that particular examples included herein are representative and not limiting. For example, those skilled in the art, reviewing the data for ipilimumab response in melanoma, as provided in detail below, represent proof of concept and establish that neoepitope mutation signatures can be predictive of response to immune checkpoint modulators. Those of ordinary skill in the art, reading the present disclosure, will appreciate and understand that the approach is broadly applicable across cancers and immune checkpoint modulator therapies.

### EXAMPLE 1

### Mutational Landscape of Melanomas from Patients with Diverse Clinical Outcomes to Ipilimumab

This example illustrates analysis of the genetic landscape of cancer, and demonstrates its effectiveness in defining useful hallmarks of patients that respond favorably or poorly to an immune checkpoint modulator. The example particularly exemplifies analysis of melanoma patients treated with CTLA-4 blockade (e.g. ipilimumab), and defines exemplary genetic characteristics in such patients.

Melanoma patients treated with CTLA-4 blocking agents demonstrate an overall survival advantage and diverse responses.^{1,27-29} Baseline patient characteristics are described in Table 3.

TABLE 3
Clinical characteristics of patients in the discovery set and validation set

| | Discovery Set | | | | Validation Set | | | |
|---|---|---|---|---|---|---|---|---|
| | Long-Term | | Minimal or | | Long-Term | | Minimal or | |
| | Benefit | | No Benefit | | Benefit | | No Benefit | |
| Total | 11 | | 14 | | 25 | | 14 | |
| Age at start of treatment | 63 | (39-70) | 59.5 | (48-79) | 66 | (33-90) | 57 | (18-74) |
| (median, range) | | | | | | | | |
| Gender (n, %) | | | | | | | | |
| F (n, %) | 3 | (27) | 8 | (57) | 9 | (36) | 5 | (36) |
| M (n, %) | 8 | (73) | 6 | (43) | 16 | (64) | 9 | (64) |
| Disease origin (n, %) | | | | | | | | |
| Acral | 0 | (0) | 3 | (21) | 1 | (4) | 1 | (7) |
| Uveal | 0 | (0) | 0 | (0) | 1 | (4) | 0 | (0) |
| Cutaneous | 10 | (82) | 8 | (57) | 15 | (60) | 11 | (79) |
| Unknown primary | 1 | (9) | 3 | (21) | 3 | (0.12) | 0 | (0) |
| Not available | 0 | (0) | 0 | (0) | 5 | (20) | 2 | (14) |
| BRAF or NRAS mutation (n, %) | | | | | | | | |
| Absent | 1 | (9) | 6 | (43) | 17 | (68) | 11 | (79) |
| Present | 10 | (91) | 8 | (57) | 8 | (32) | 3 | (21) |
| LDH at start of therapy (n, %) | | | | | | | | |
| Normal | 8 | (73) | 8 | (57) | 8 | (32) | 9 | (64) |
| Above normal | 2 | (18) | 5 | (33) | 3 | (12) | 3 | (21) |
| Not available | 1 | (9) | 1 | (7) | 14 | (56) | 2 | (14) |
| Duration of response | 59 | (42-361+) | 14 | (11-23) | 130 | (64-376) | 11 | (3-29) |
| (median weeks, range) | | | | | | | | |
| Prior therapies | 1 | (0-3) | 1 | (0-2) | 0 | (0-2) | 0 | (0-3) |
| (median number, range)* | | | | | | | | |
| Stage at Diagnosis (n, %) | | | | | | | | |
| IIIC | 0 | (0) | 0 | (0) | 3 | (12) | 0 | (0) |
| M1a | 0 | (0) | 1 | (7) | 4 | (16) | 1 | (7) |
| M1b | 5 | (45) | 1 | (7) | 2 | (8) | 3 | (21) |
| M1c | 6 | (55) | 12 | (86) | 16 | (64) | 10 | (71) |
| Overall Survival | 4.4 | (2-6.9) | 0.9 | (0.4-2.7) | 3.3 | (1.6-7.2) | 0.8 | (0.2-2.1) |
| (median years, range) | | | | | | | | |

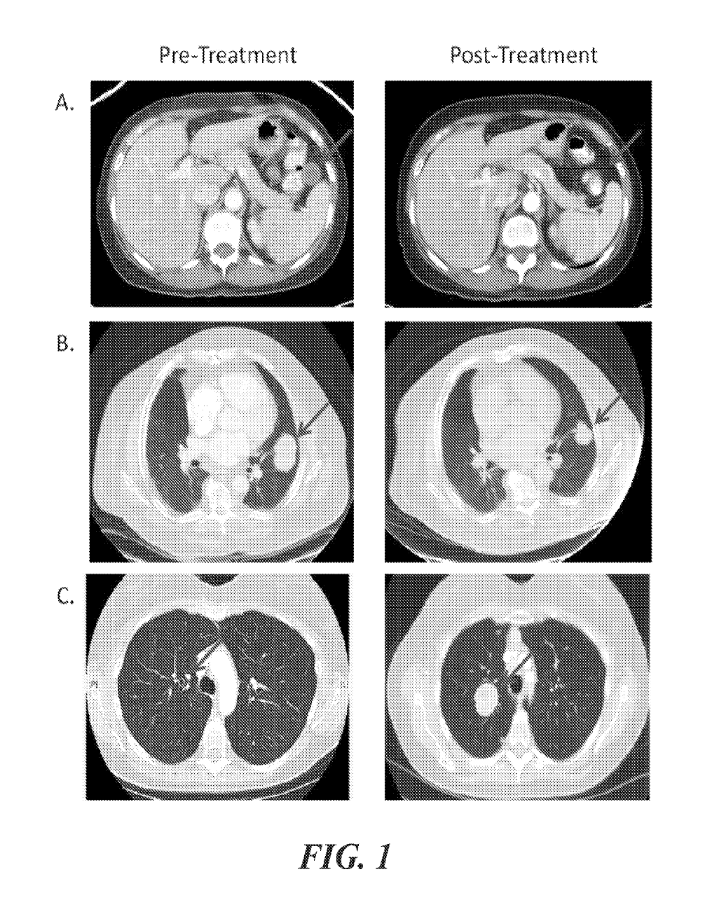

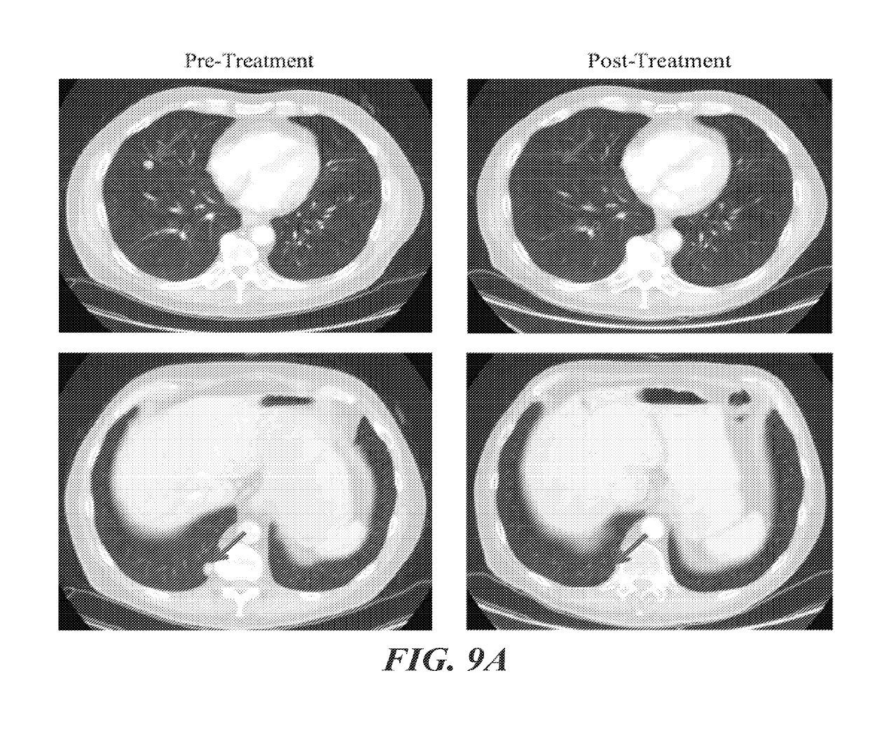

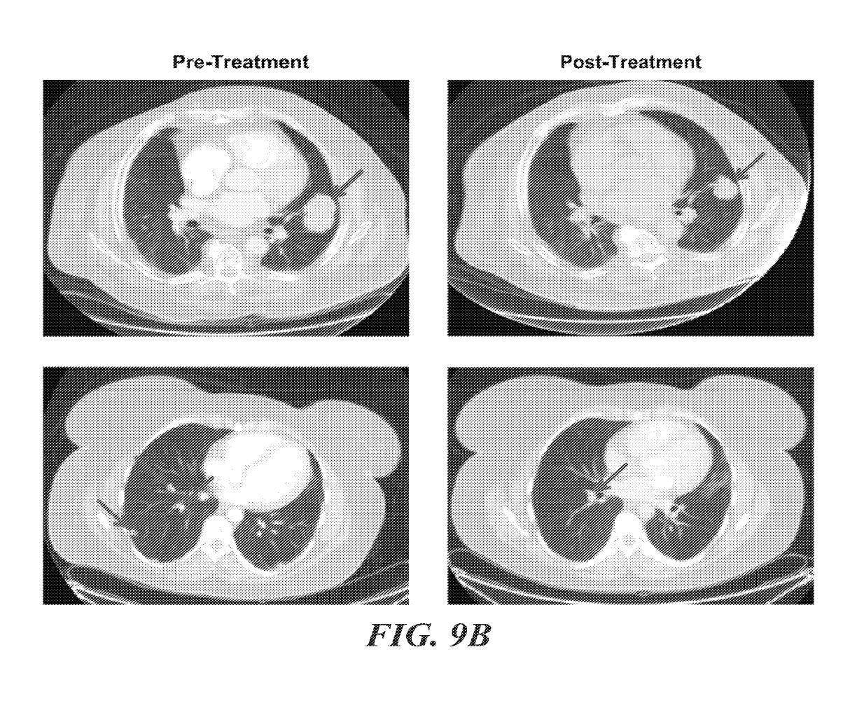

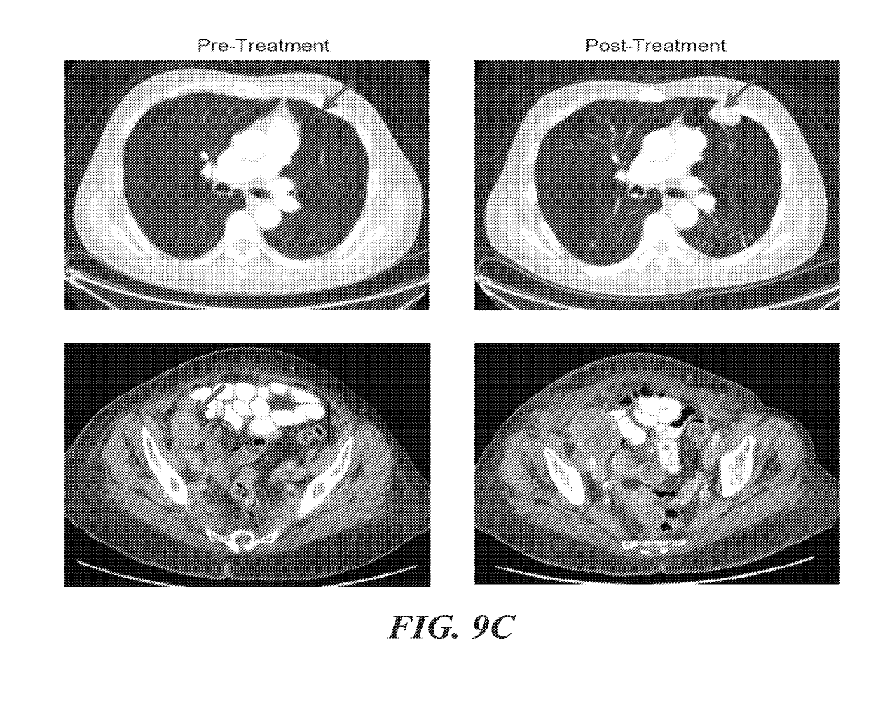

Included in this study were patients with or without long-term clinical benefit. Here, we define long-term clinical benefit as either (1) patients radiographically free of disease (NED) (from CTLA-4 blocking agents alone or with resection of an isolated stable or non-responding lesion); or (2) patients with evidence of stable or decreased volume of disease for >6 months. We define absence of clinical benefit as tumor growth at every scan after the initiation of treatment (no benefit or response), or temporary clinical benefit or response lasting <6 months (minimal benefit) (representative scans, FIG. 1A-C and FIGS. 9A-C).

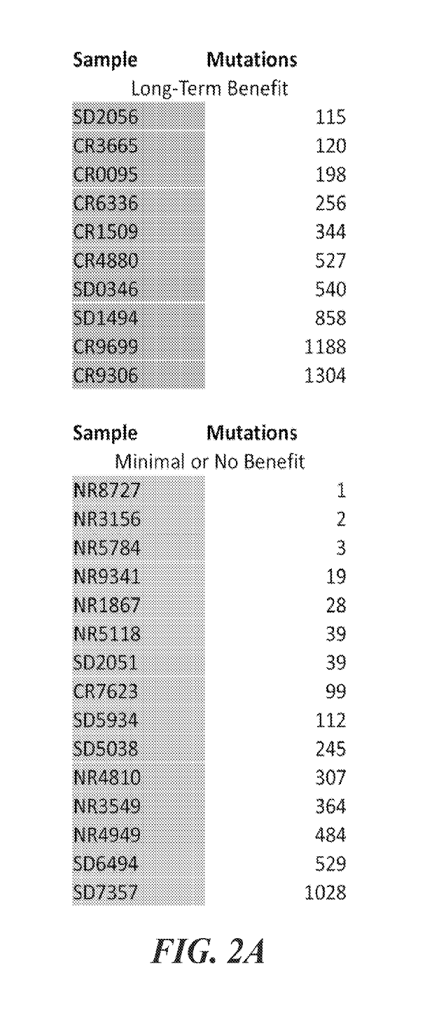

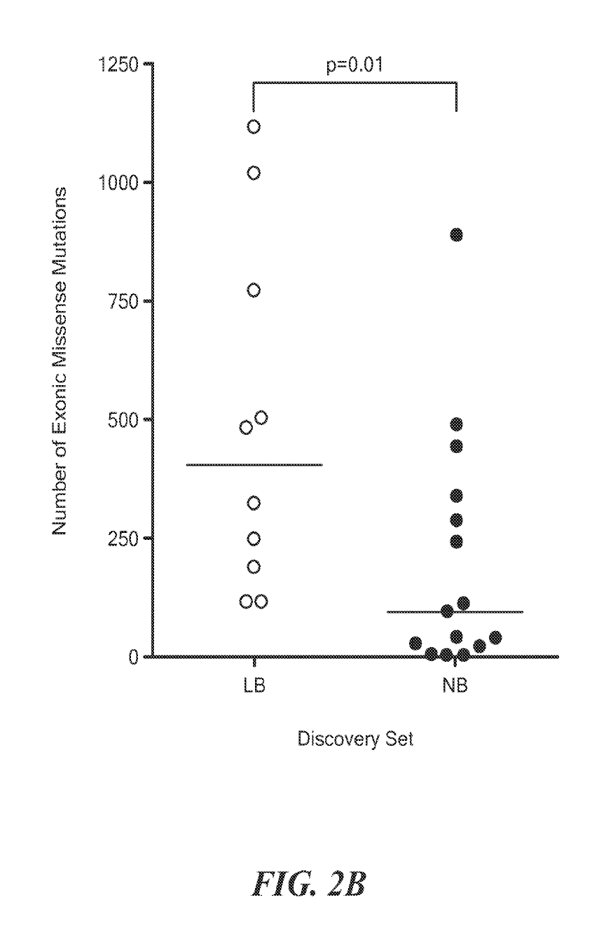

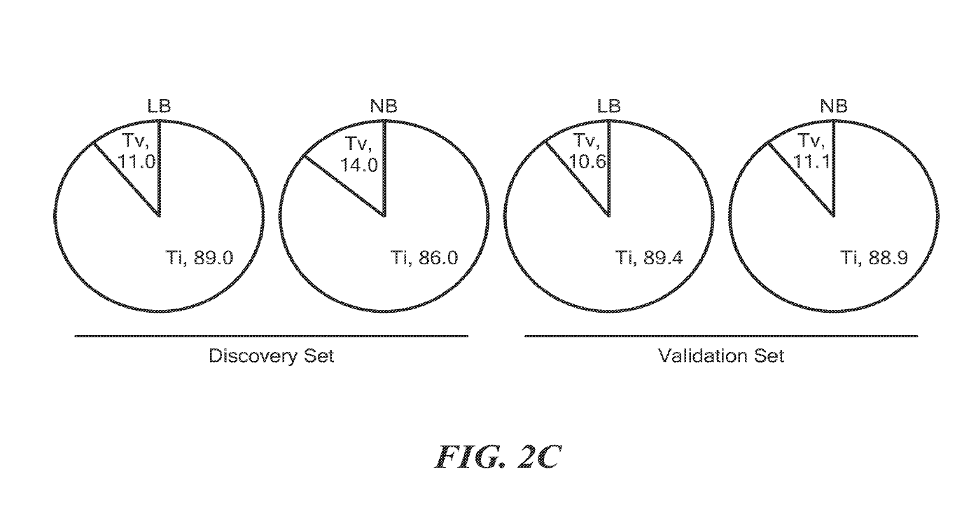

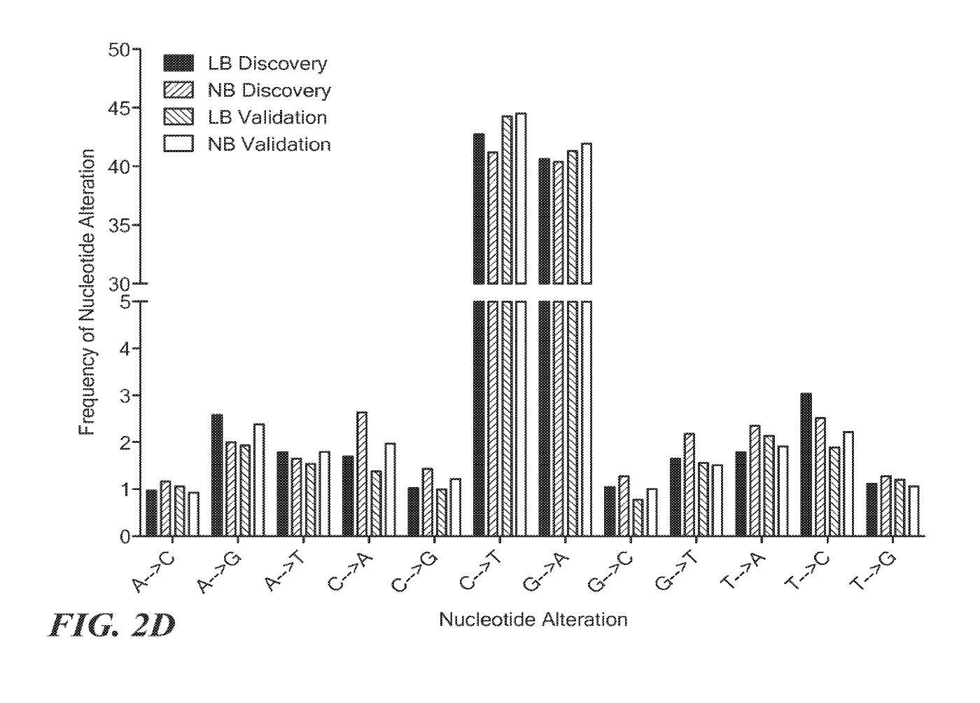

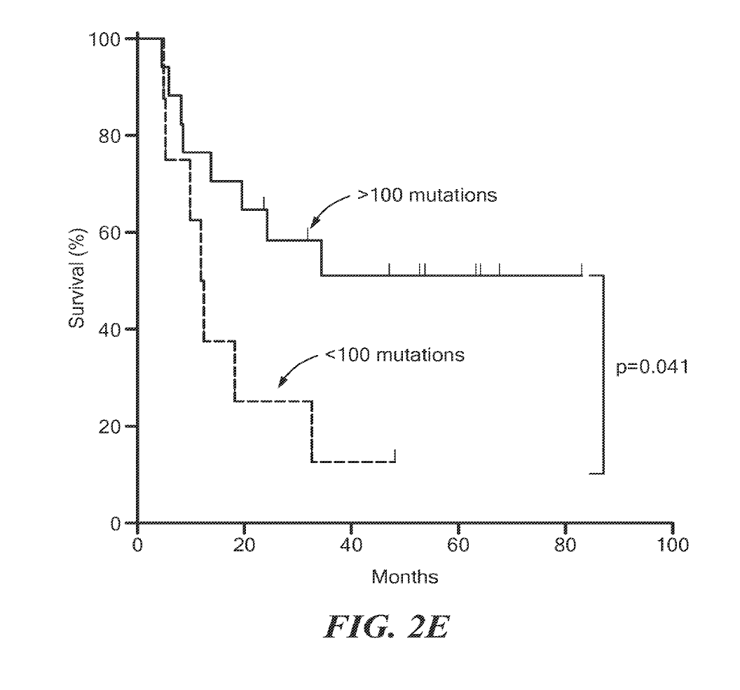

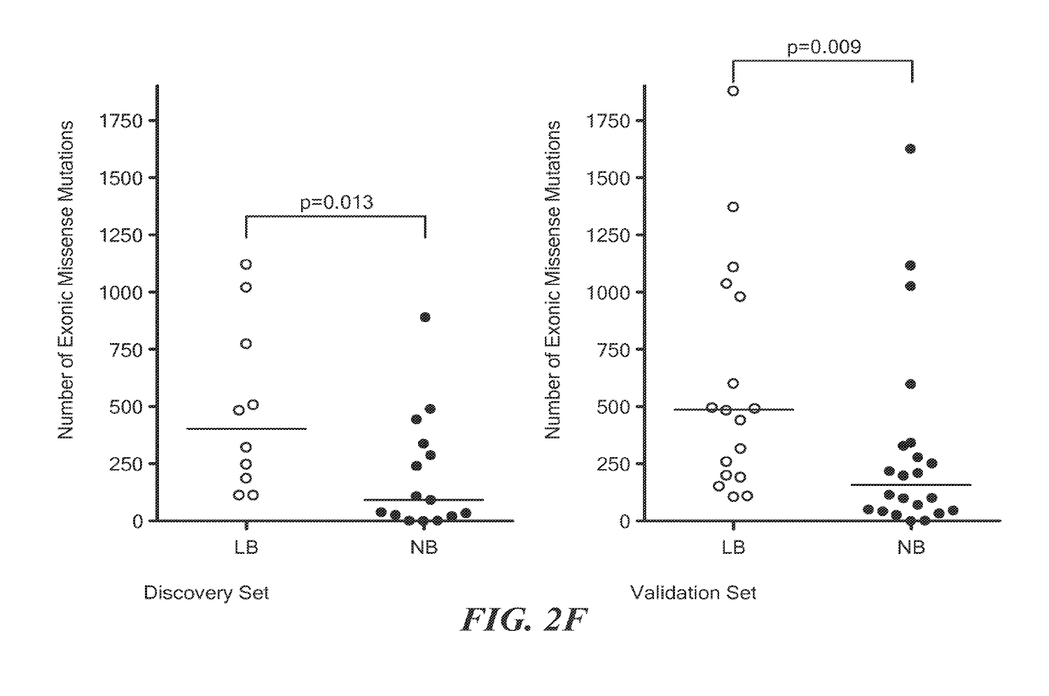

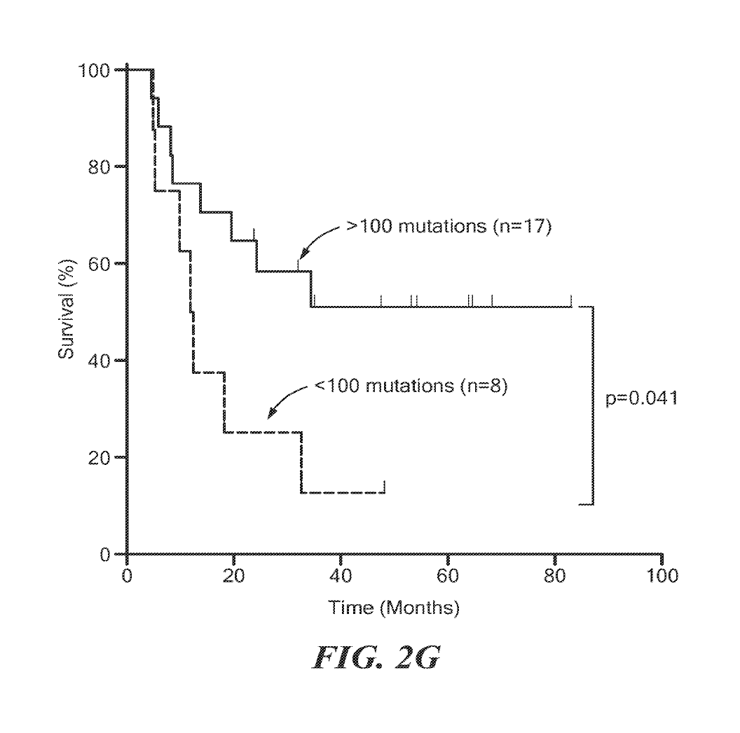

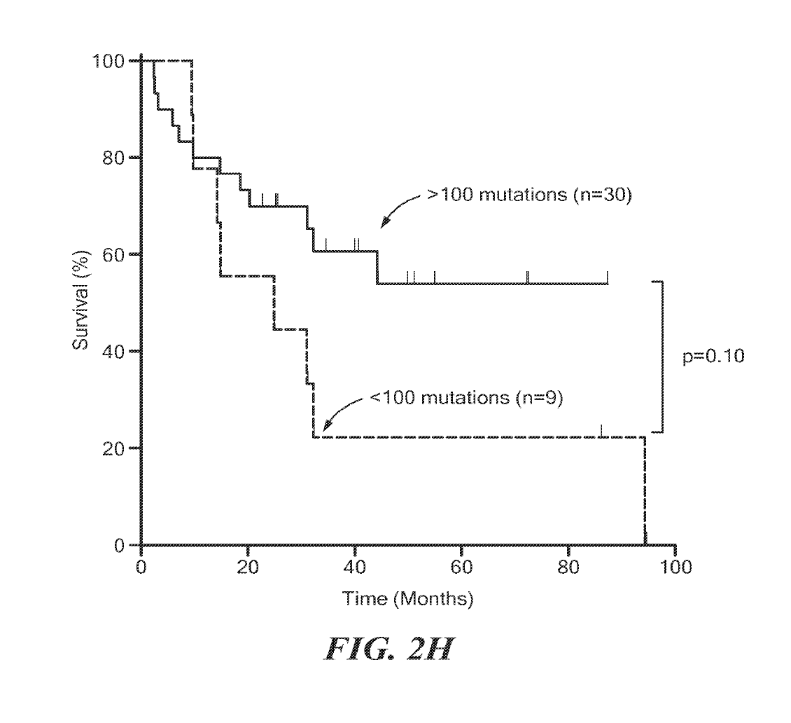

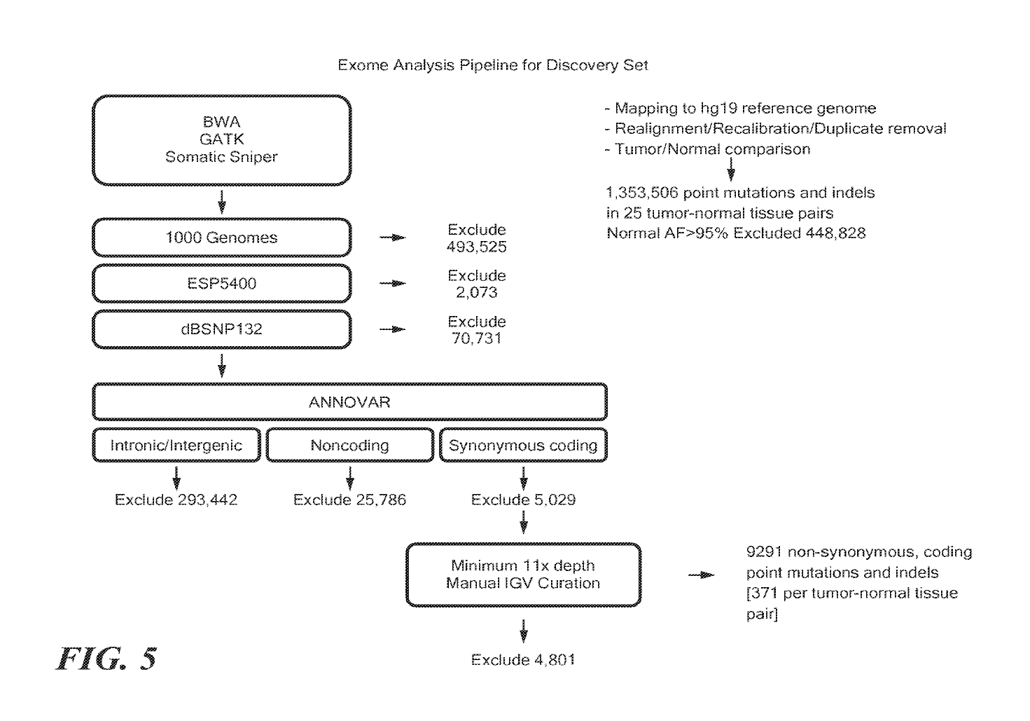

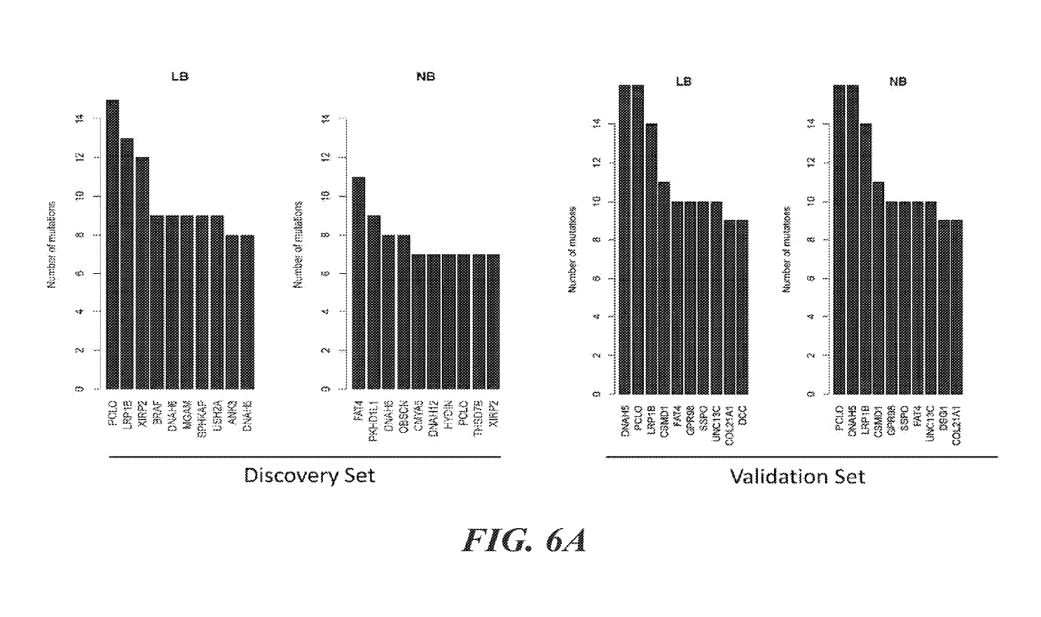

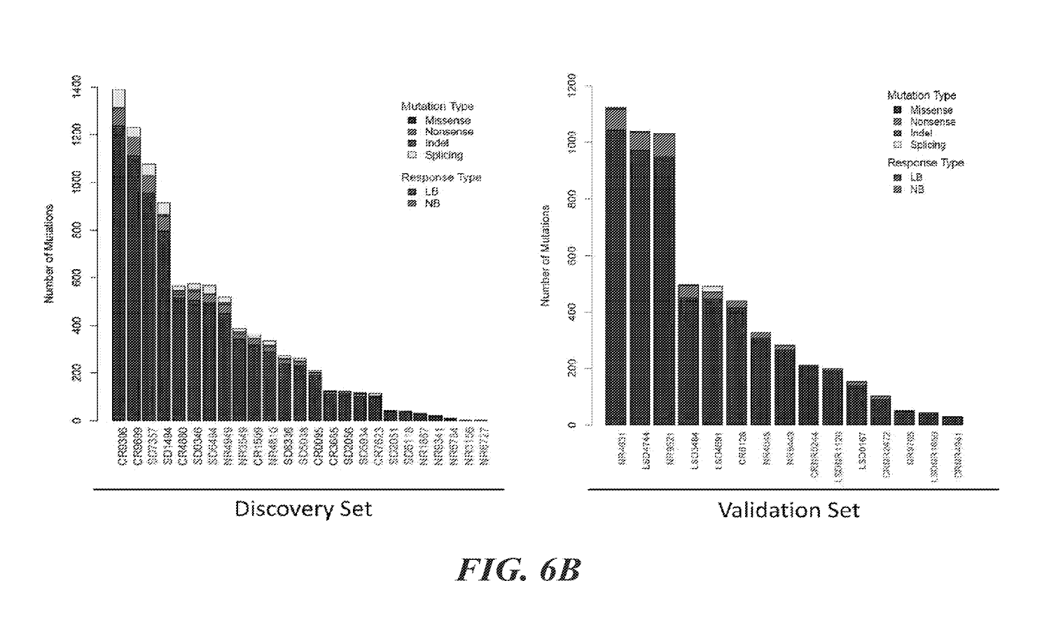

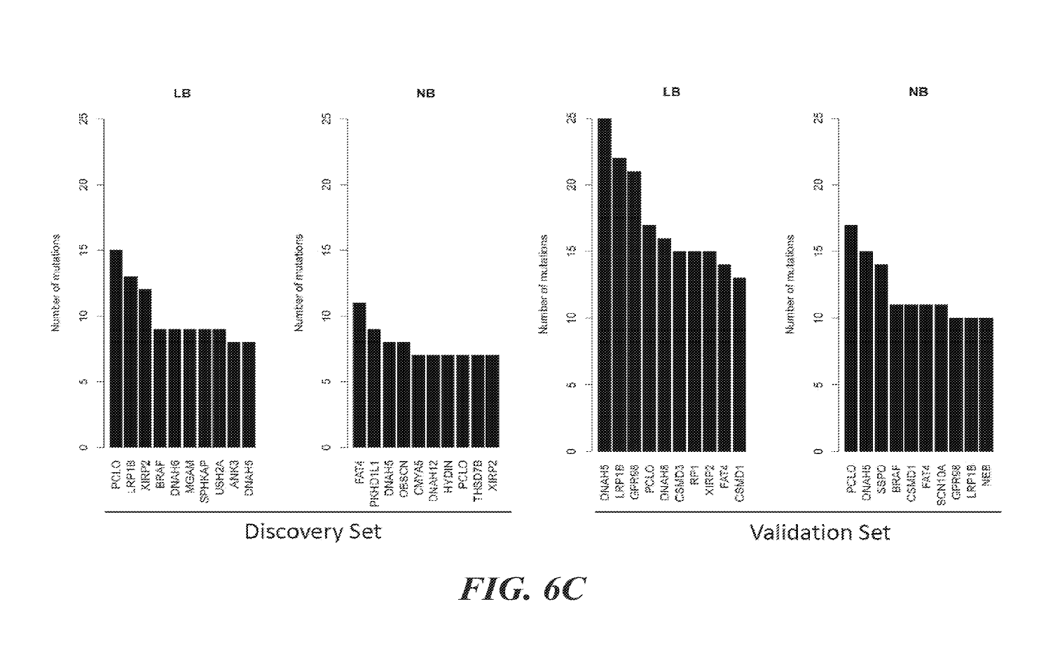

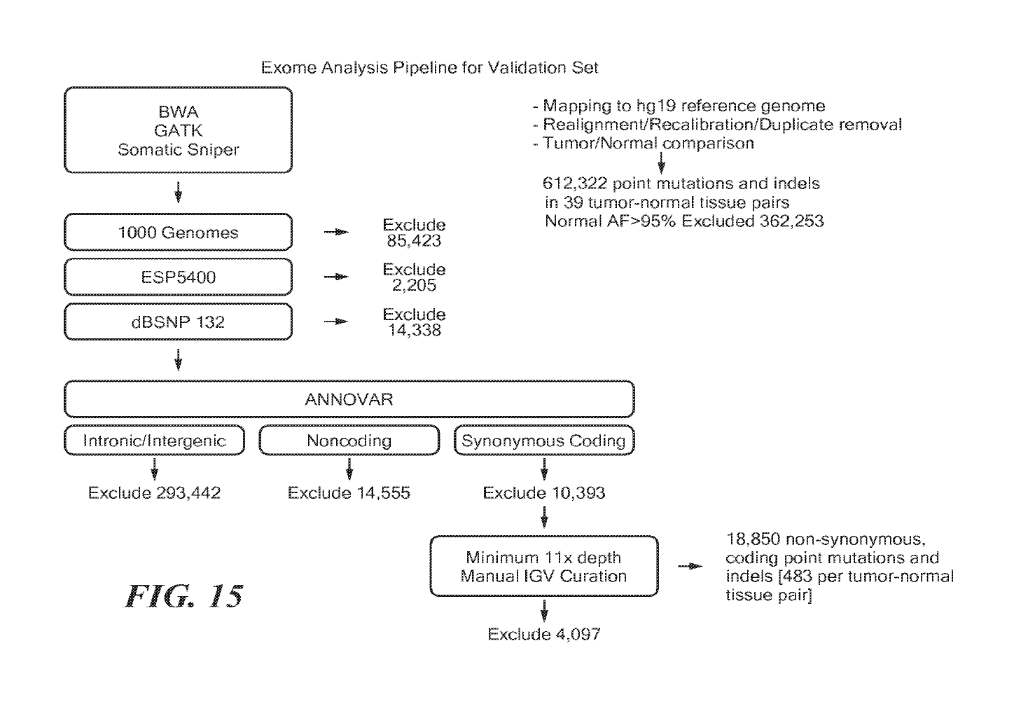

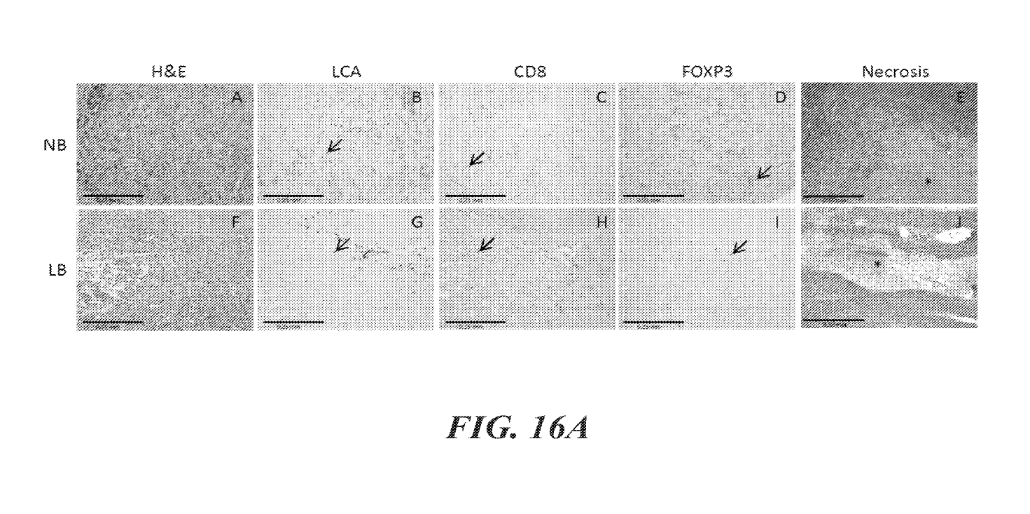

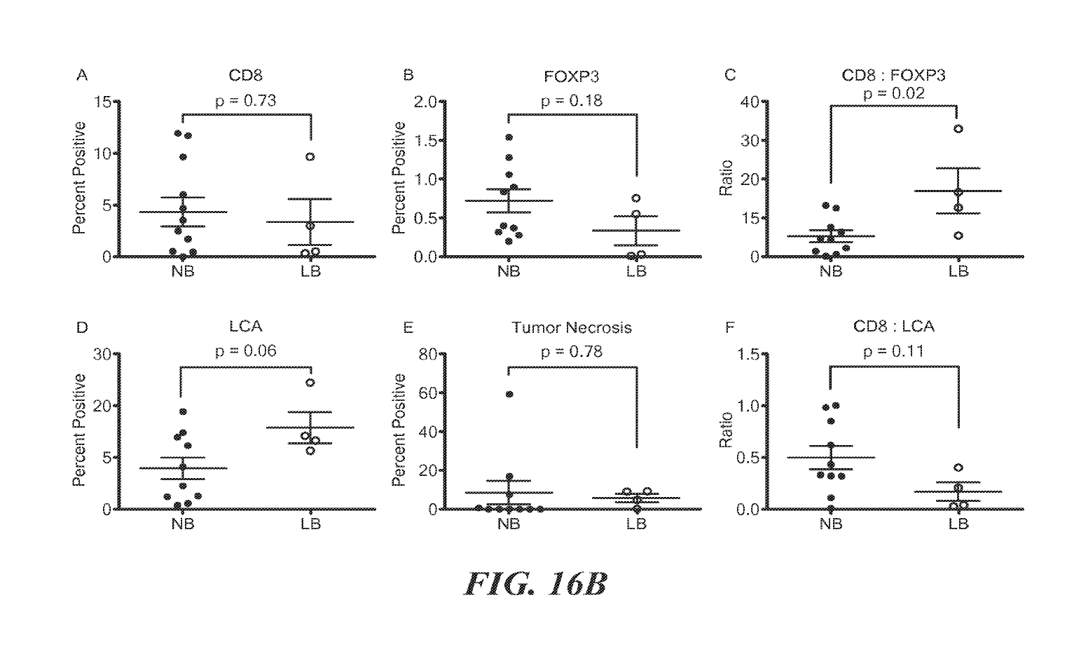

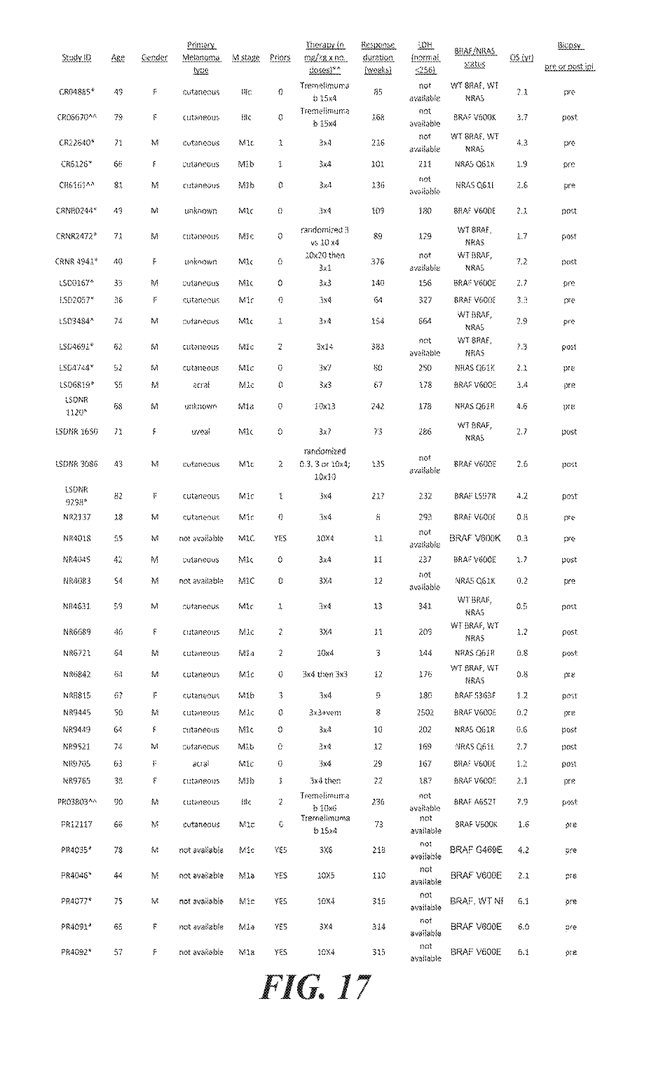

To determine the genetic landscape of response from CTLA-4 blocking agents, we analyzed tumor and matched blood DNA using whole exome sequencing. In the discovery set, we generated 6.4 GB of mapped sequence, with over 90% of the target sequence covered to at least 10× depth and mean exome coverage of 103× (FIG. 5). The results of a validation set are depicted in FIG. 15. The wide range of mutational burdens among samples (FIGS. 2A and 2B) and recurrent and driver mutations (FIGS. 6A and 6C), were consistent with the literature.³⁰⁻³⁴

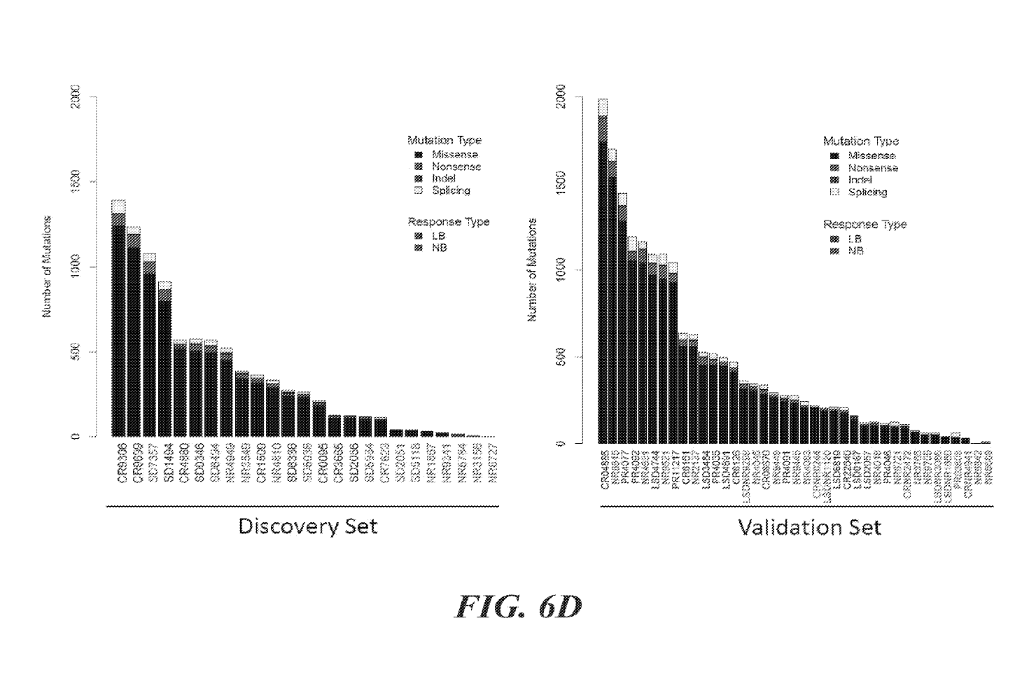

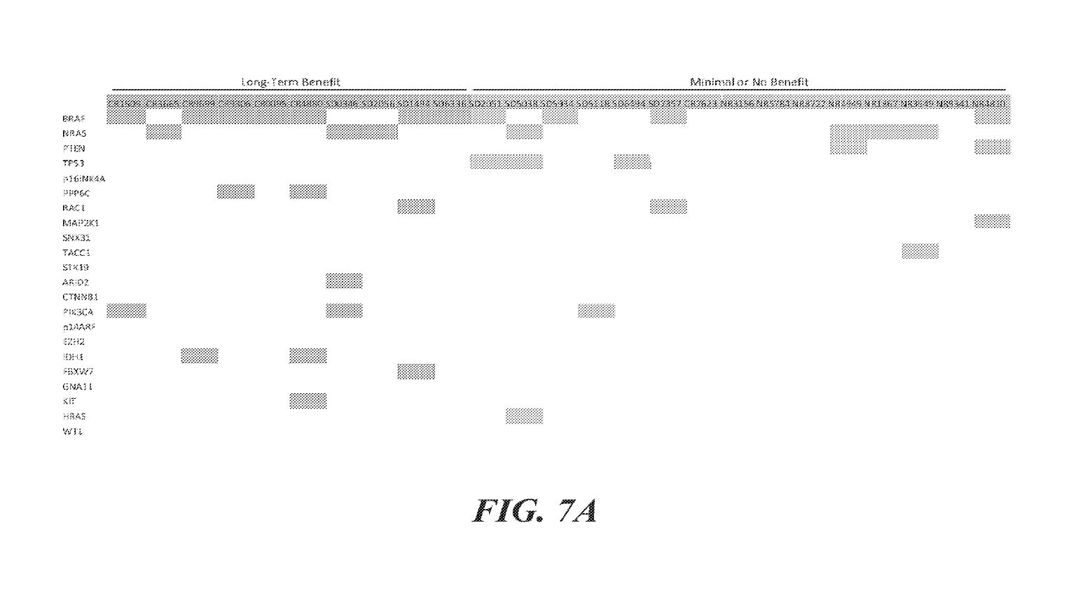

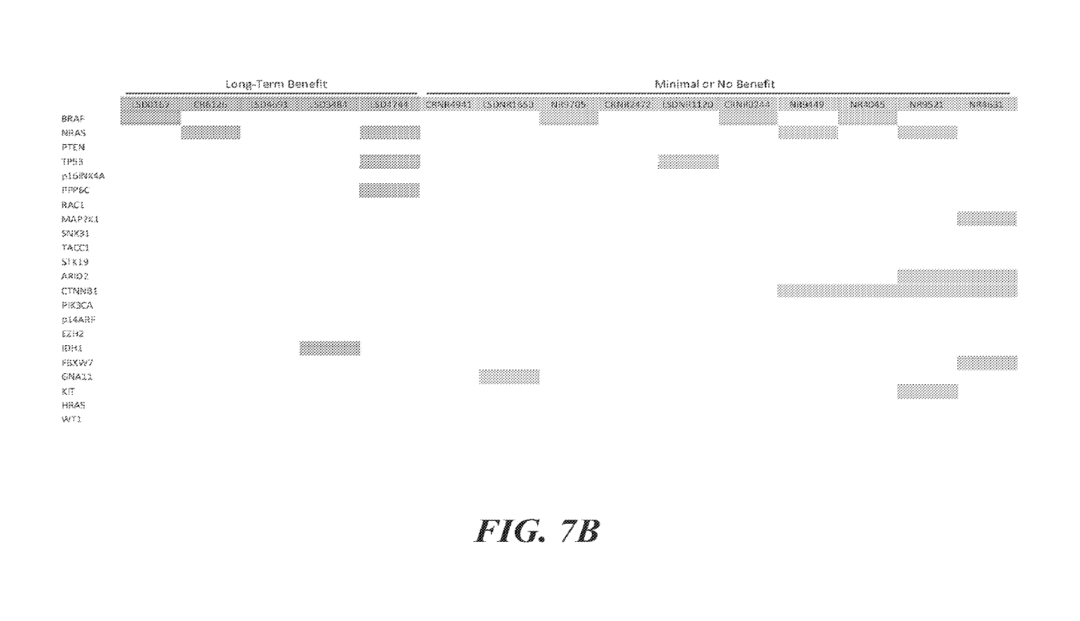

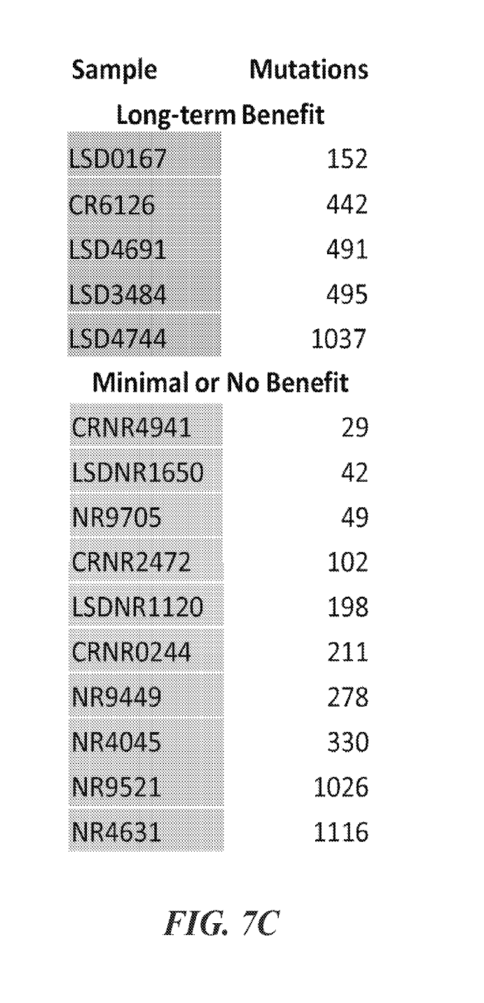

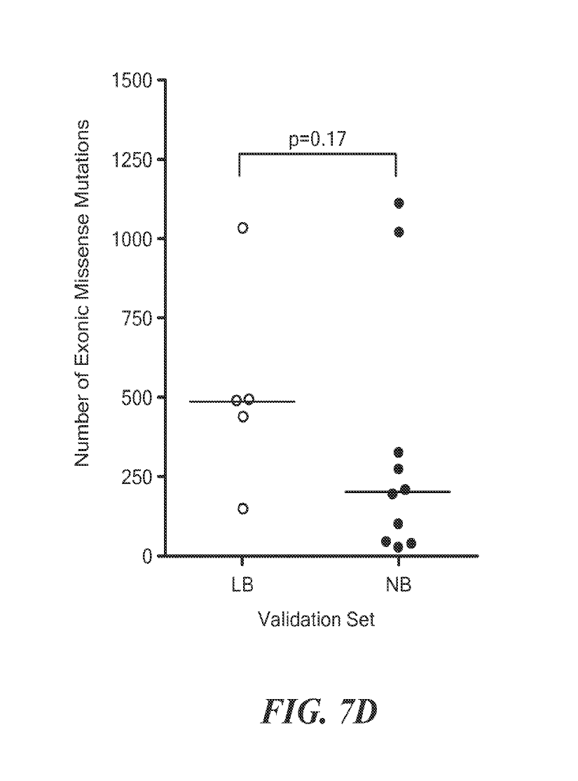

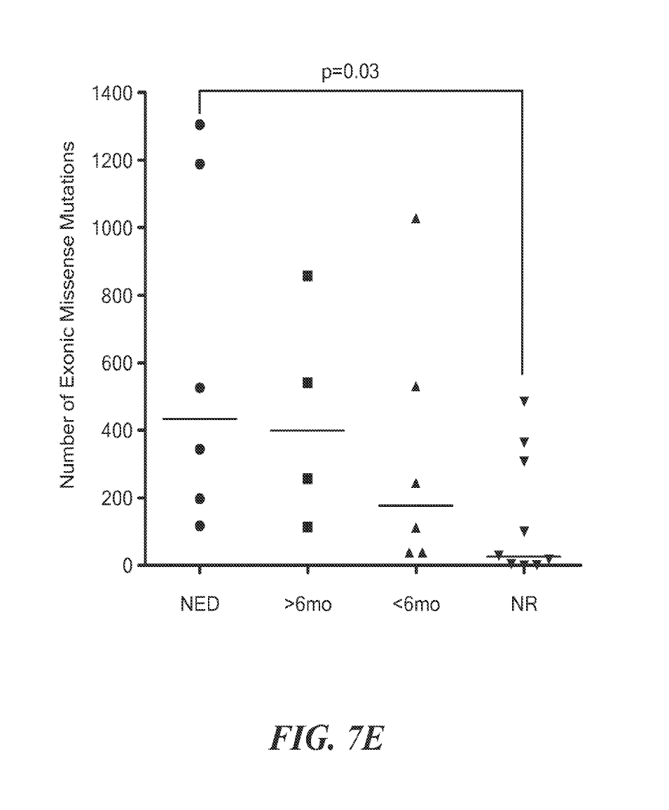

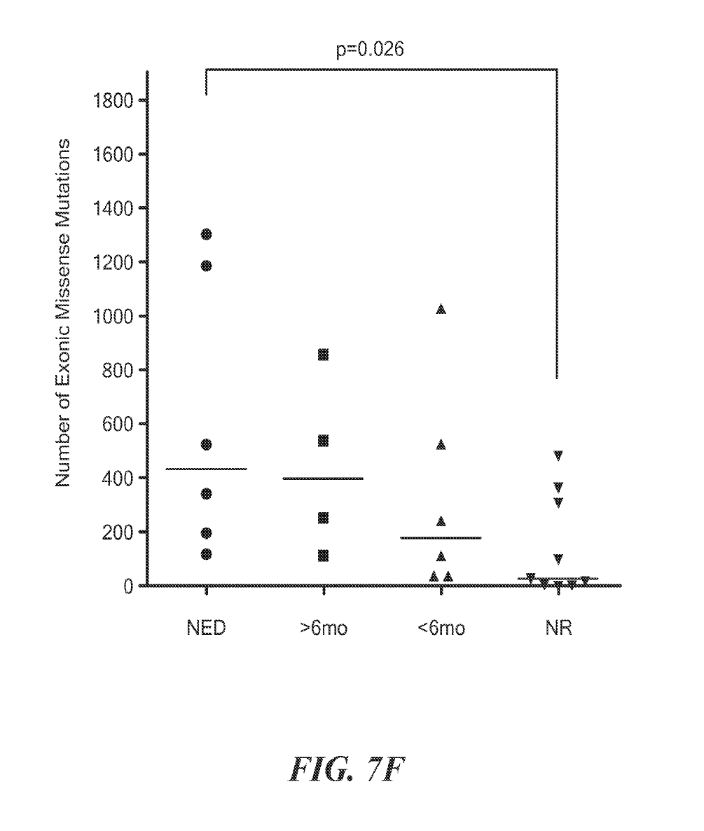

In discovery and validation sets, there was a similar ratio of transitions to transversions (FIGS. 2C, 2I), as well as mutation types and nucleotide changes (FIG. 2D and FIGS. 6B and 6D).¹⁹ No gene was universally mutated across responders or patients who derived benefit. Mutations in known, recurrent melanoma driver genes were observed in each group (FIGS. 7A and 7B) and responses were seen in melanomas with a diversity of driver mutations.³⁵

### EXAMPLE 2

### Somatic Neoepitopes Associated with Treatment Efficacy

This example demonstrates that somatic neoepitopes are associated with efficacy of treatment with an immune checkpoint modulator and, among other things, defines a neoepitope signature linked to response to a particular exemplary modulator (i.e., ipilimumab).

Mutational Burden Correlates with Clinical Benefit but Alone is not Sufficient to Predict Outcome

We hypothesized that increased mutational burden in metastatic melanoma samples might correlate with response to CTLA-4 blockage (e.g., to treatment with agents such as ipilimumab, tremelimumab, etc). There was a significant difference in mutational load between patients with long-term clinical benefit (LB) versus minimal or no clinical benefit (NB) from CTLA-4 blocking agents in the discovery set (FIG. 2B, Mann Whitney test, p=0.013), and in the validation set (FIGS. 7C and 7D, Mann Whitney test, p=0.009). In the discovery set, mutation load correlated with improved overall survival (FIG. 2E, Log-Rank test, p=0.041) and trended towards improved survival in the validation set (FIG. 2E, and FIG. 2H). The latter set included eight non-responding tumors resected from patients who otherwise achieved systemic disease control, which may confound the realtionshipo between mutational load and survival. Further subdivision into four clinical categories was suggestive of a dose-response in the discovery set (FIG. 7E). These data indicate that a high mutational load correlates with clinical benefit from CTLA-4 blocking agents (e.g. ipilimumab), but alone is not sufficient to impart a clinical response, as there are tumors with high mutational burden that did not respond.

Somatic Neoepitopes Common to Responding Tumors are Associated with Anti-CTLA-4 Efficacy

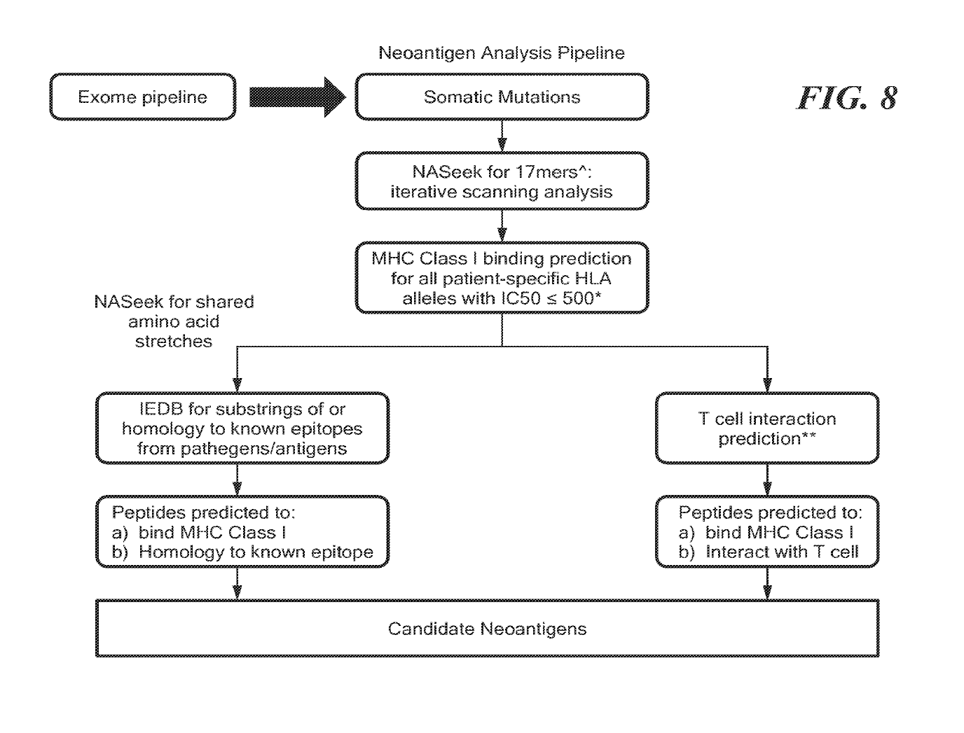

MHC class I presentation and cytotoxic T-cell recognition are required for ipilimumab activity.¹⁵ Since mutational load alone did not explain clinical response to ipilimumab, we hypothesized that the presence of specific tumor neoantigens might explain the varied therapeutic response. To identify such neoepitopes, a state-of-the-art bioinformatic pipeline was developed incorporating MHC class I binding prediction, modeling of T cell receptor binding, patient-specific HLA type and epitope homology analysis (FIG. 8 and Methods).

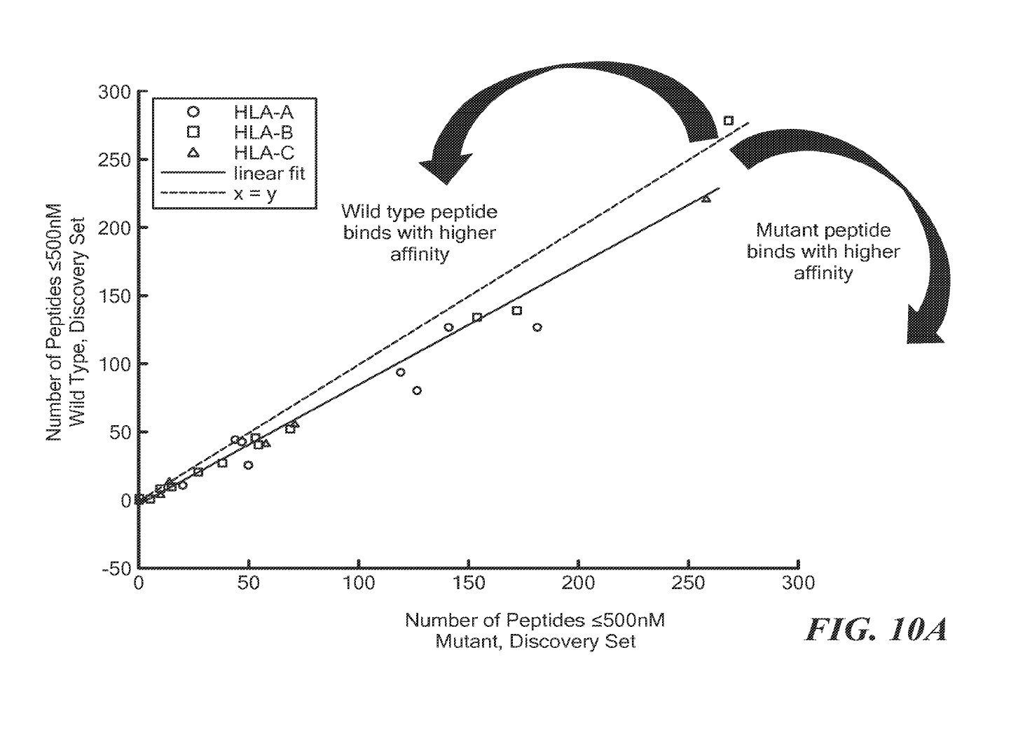

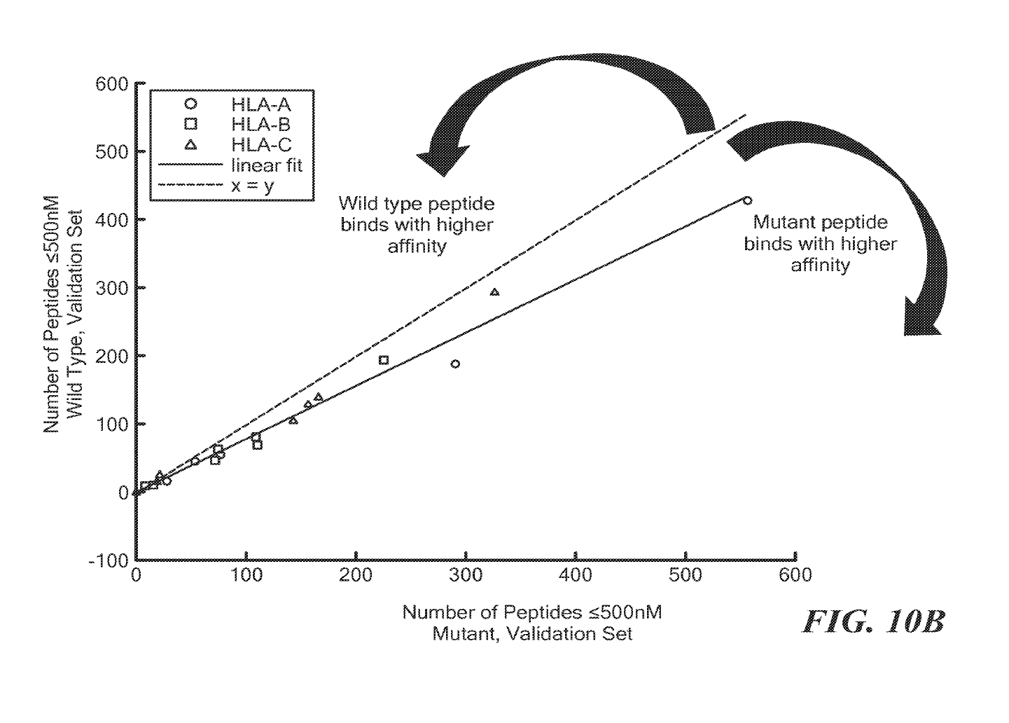

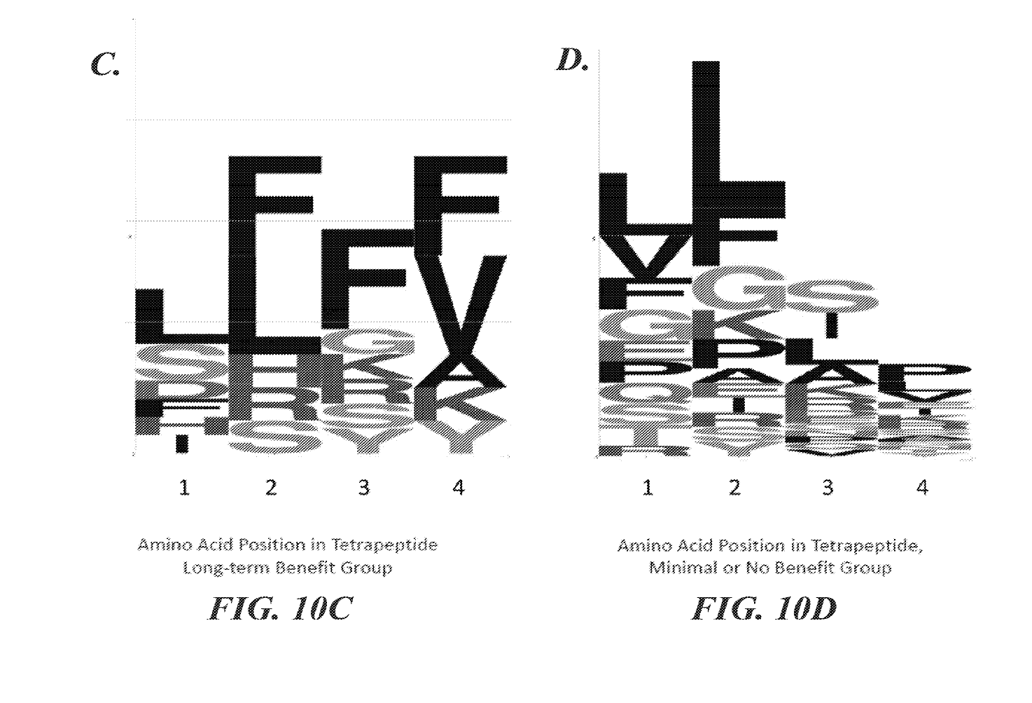

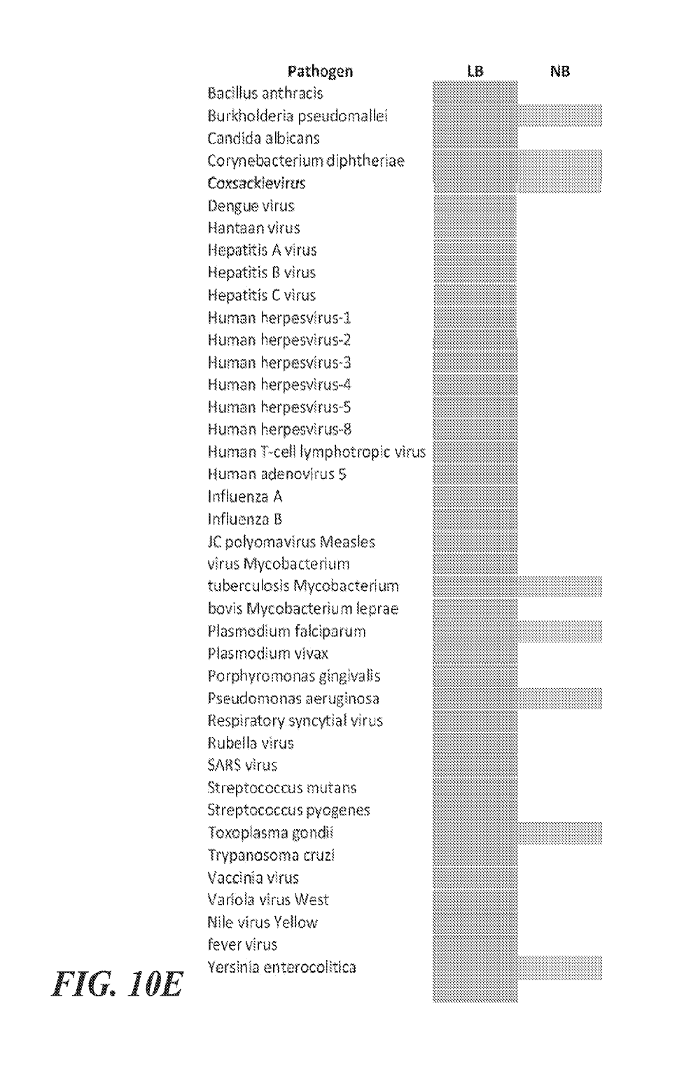

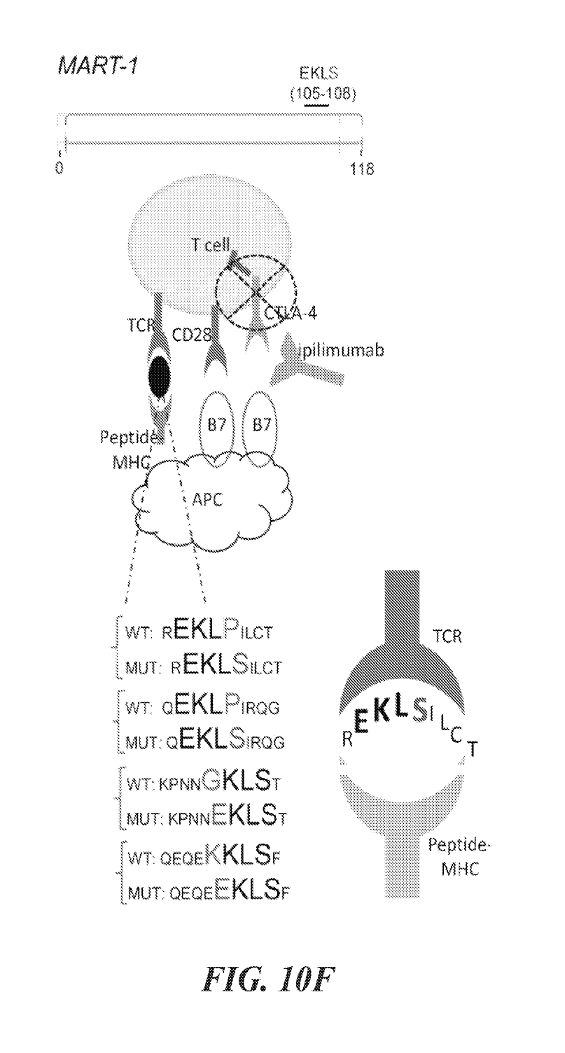

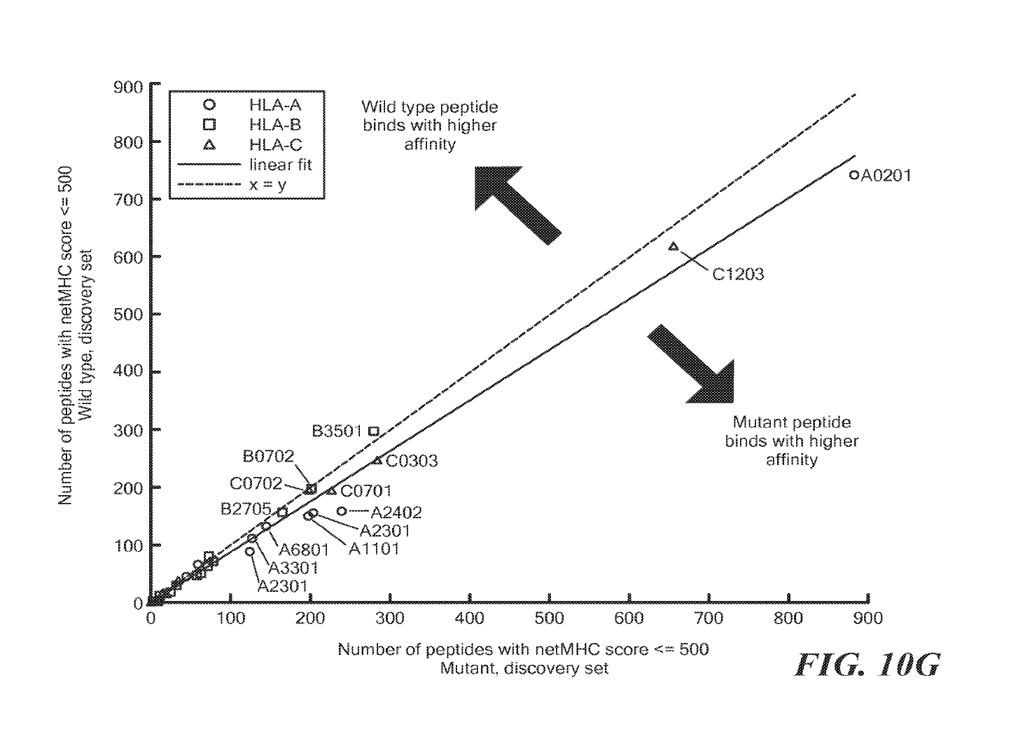

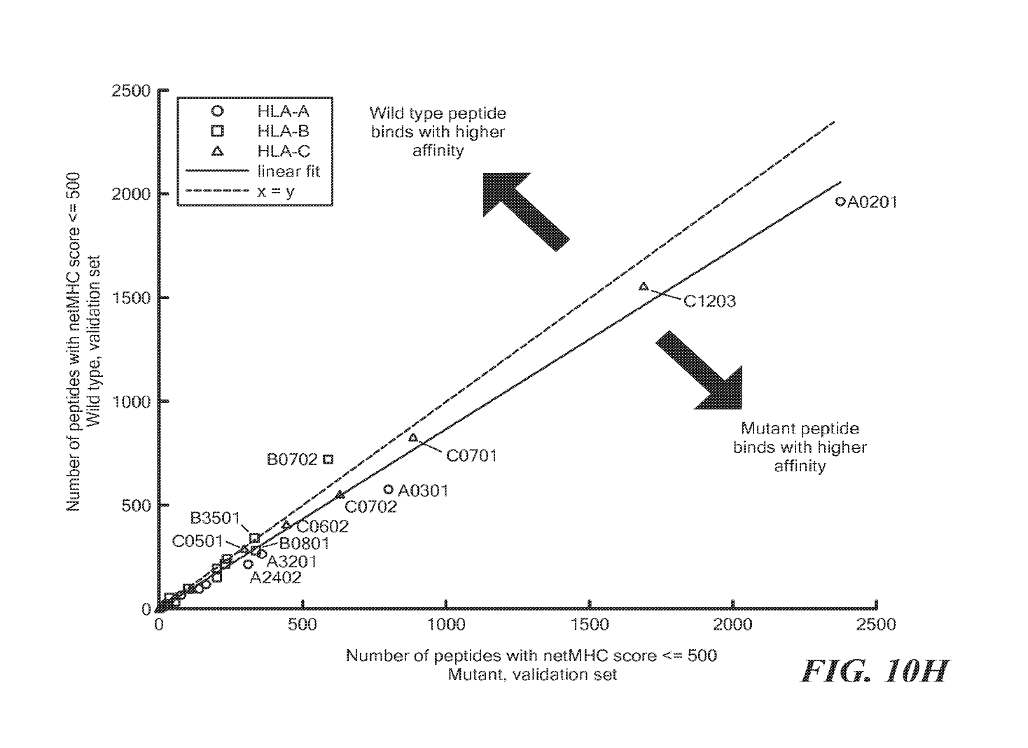

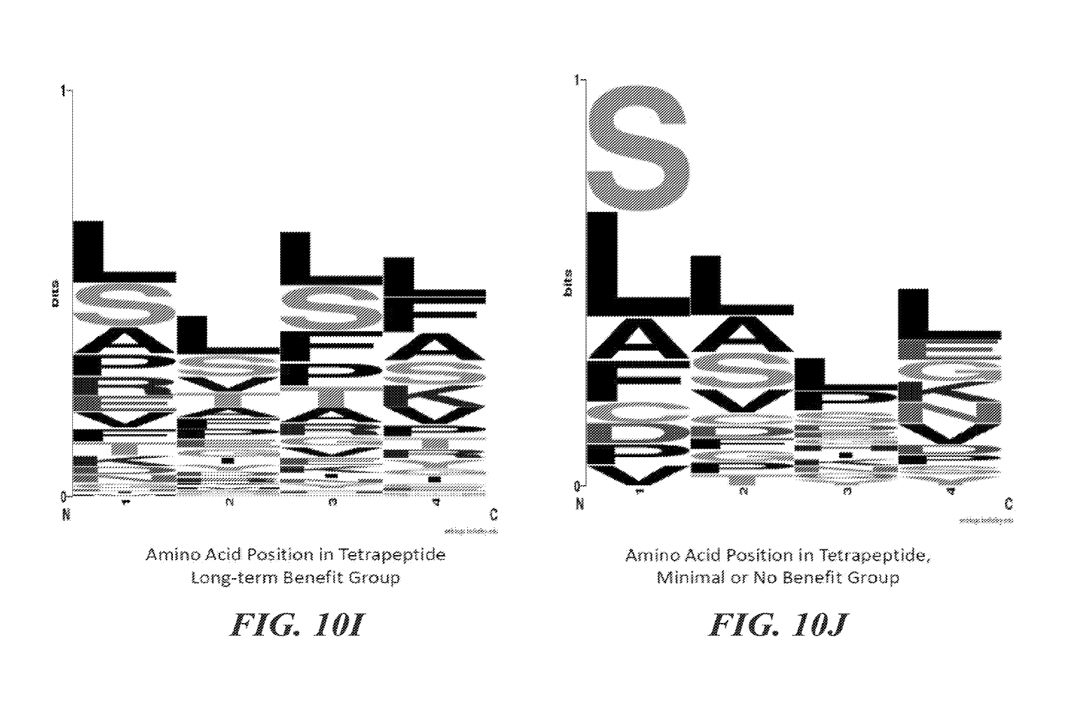

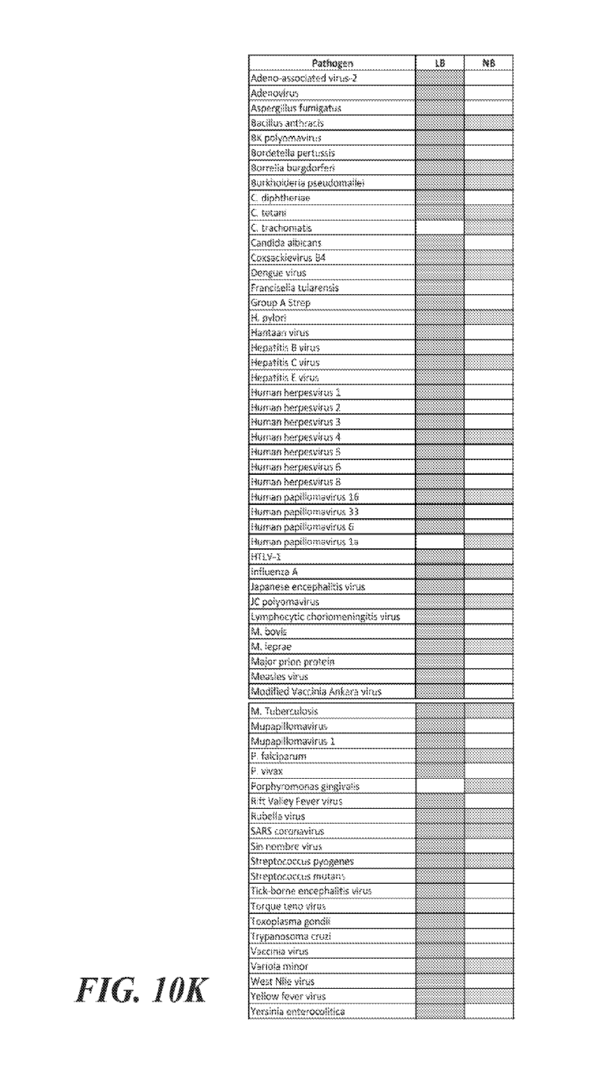

Tumor antigen presentation by MHC Class I is critical for recognition by T cells.^{36,37} We created a computational algorithm to translate all nonsynonymous missense mutations into mutant and wild type peptides (NASeek, Methods, and FIG. 8). We examined whether a subset of somatic neoepitopes would alter the strength of peptide-MHC binding, using patient-specific HLA types. We first compared the overall antigenicity trend of all mutant versus wild type peptides. Intriguingly, in aggregate, the mutant peptides were predicted to bind MHC Class I with higher affinity than the corresponding wild type peptides (FIGS. 10A and 10B, FIGS. 10F and 10G).

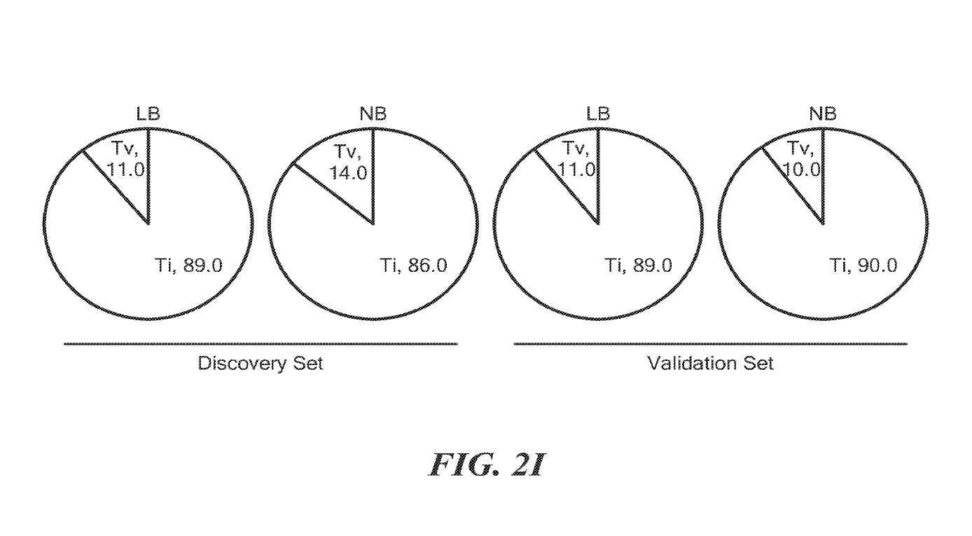

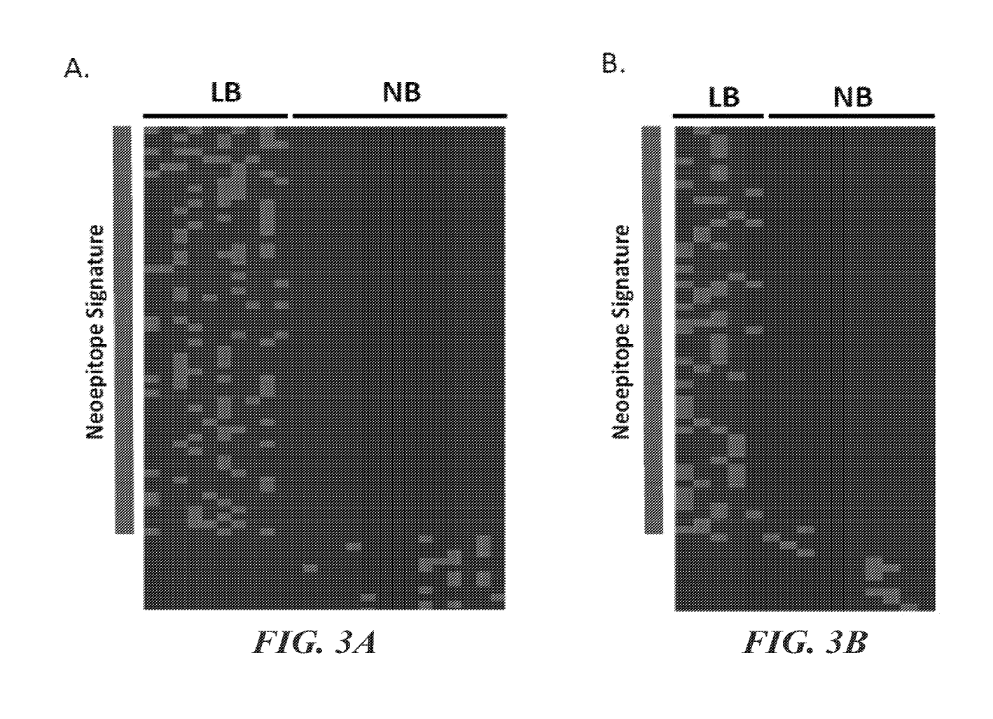

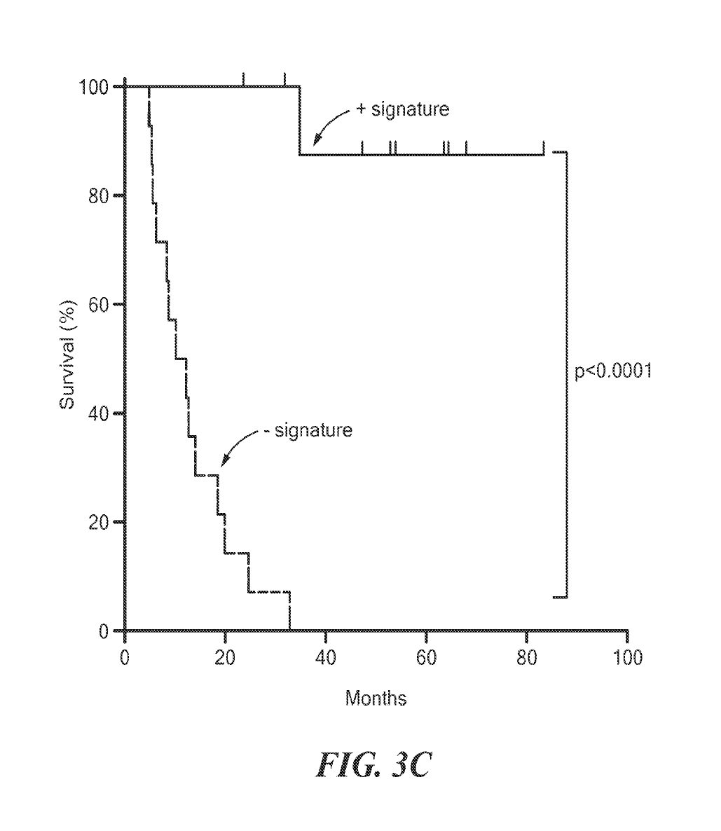

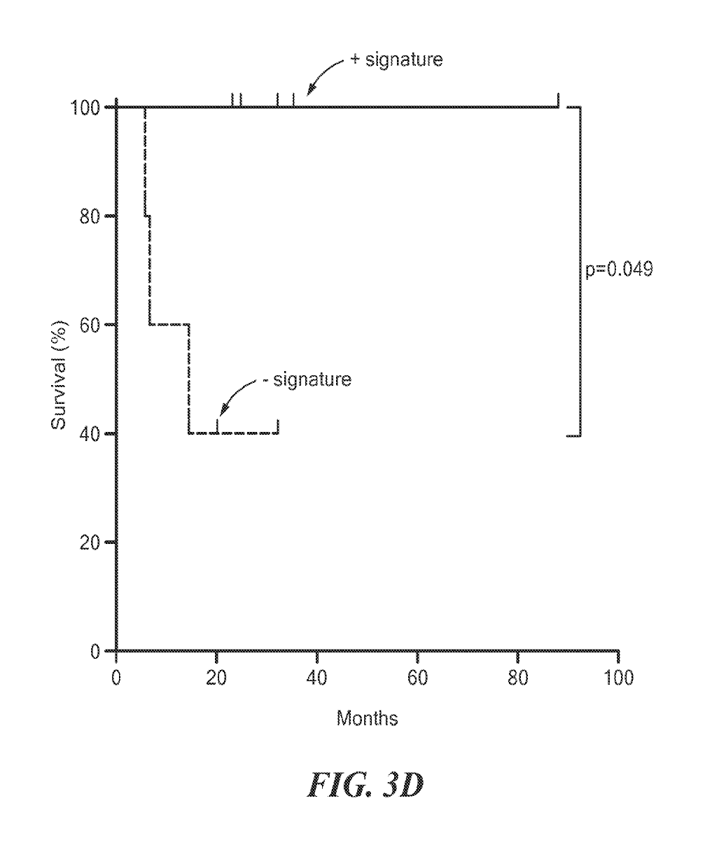

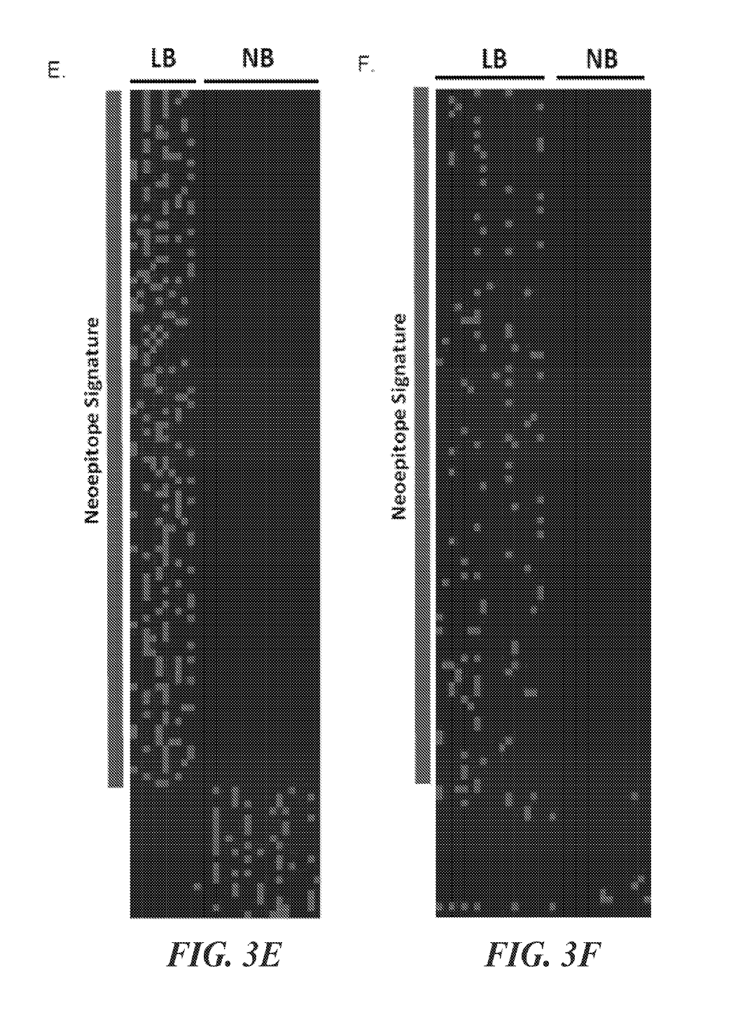

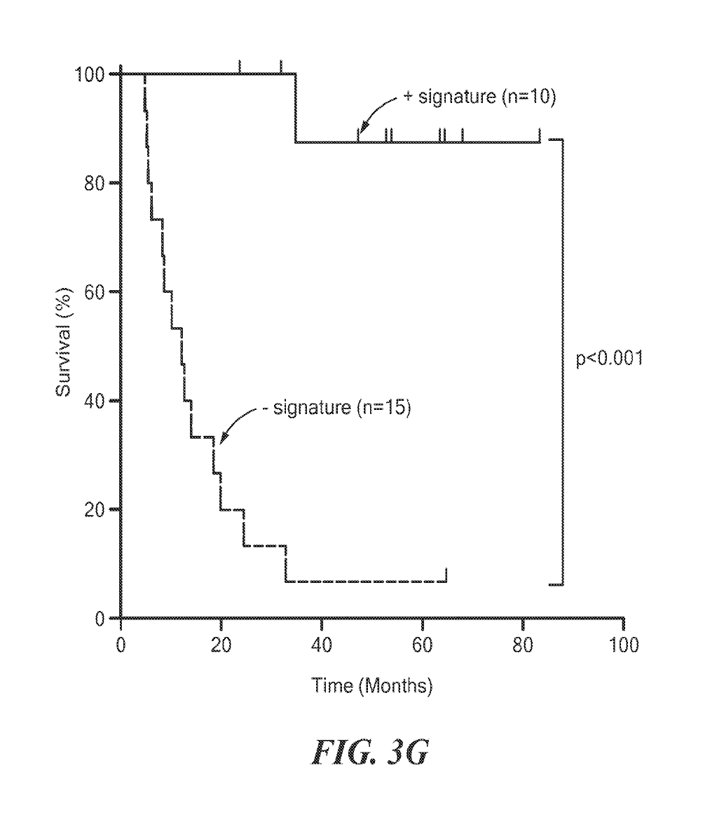

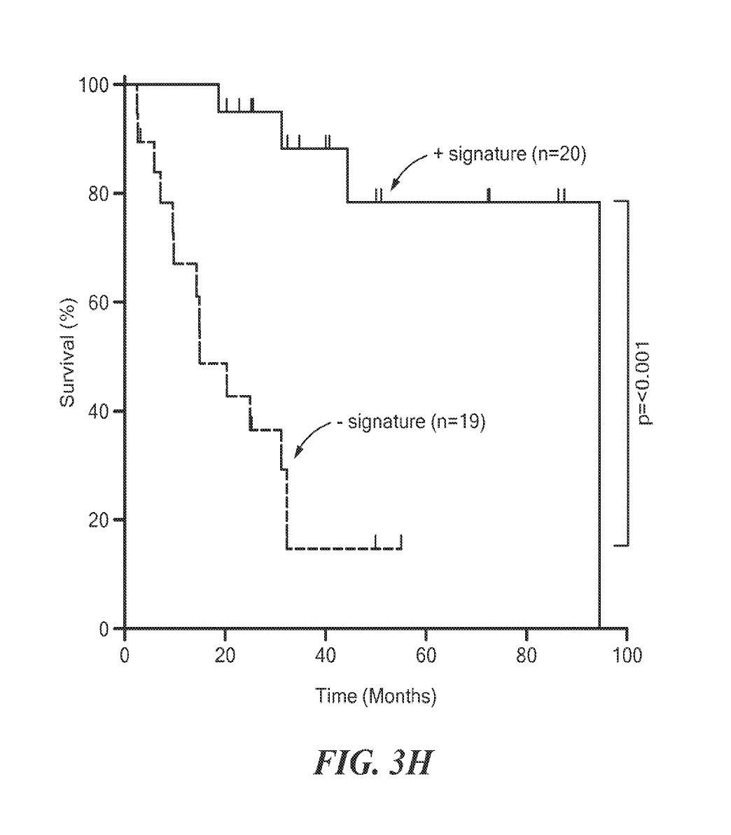

Using only peptide strings predicted to bind to MHC Class I (IC50≦500 nM), we searched for conserved stretches of amino acids shared by multiple tumors, focusing on tetrapeptides. These are used in modeling genome phylogeny because they occur relatively infrequently in proteins and typically reflect function.³⁸ We used standard machine learning, hierarchical clustering, and signature derivation approaches to identify consensus sequences. We identified a number of tetrapeptide sequences shared by responders but completely absent from nonresponders. (FIGS. 3A and 3B). In a recently published landmark paper, short amino acid substrings were shown to comprise conserved regions across antigens recognized by a T-cell receptor (TCR).³⁹ TCR recognition of epitopes was driven by consensus tetrapeptides, and tetrapeptides within cross-reacting TCR epitopes were necessary and sufficient to drive antigenicity and T-cell proliferation. There is strong evidence that this polypeptide length is sufficient to drive recognition by TCRs.⁴⁰⁻⁴²

Tetrapeptides can form the core of nonapeptides presented by MHC class I molecules to T cells, or may be located laterally.⁴³ Tetrapeptides are used in modeling genome phylogeny because they occur relatively infrequently in proteins and typically reflect function. We used the discovery set to generate a predictive signature from the candidate neoepitopes. The tetrapeptides common to each group (candidate neoepitopes) included 101 shared exclusively among patients with clinical benefit in the discovery set. This was also independently observed in the validation set (FIGS. 3A, 3B, 3E and 3F and FIG. 12). This set defines a neoepitope signature linked to benefit from CTLA-4 blockade (e.g., via ipilimumab) (FIGS. 3A and 3B, red line) that was highly statistically significant (p<0.001, Fisher's Exact test).

Importantly, shared tetrapeptide neoepitopes did not simply result from a higher mutational load. For example, in the discovery set, the NB patient (nonresponder) with the greatest number of mutations (SD7357 with 1028 mutations) did not share any of the tetrapeptide signature (FIG. 3A). This concept was illustrated again in the validation set in which even tumors with greater than 1000 mutations (NR9521 and NR4631) did not respond (FIG. 3B and FIG. 7C). Simulation testing using five different models demonstrated that our signature was highly statistically significant and unlikely to have resulted by chance alone (p<0.001 for methods a-d and p+0.002 for method e) (FIG. 12). A high mutational load appeared to increase the probability but not guarantee formation of a neoepitope associated with benefit. Consensus analysis revealed that the neoepitopes were not random. Frequencies of amino acids that make up the tetrapeptides in the benefitting group were different from those observed in the nonbenefitting group (FIGS. 10C, 10D, 10I and 10J).

Neoepitope signatures derived from the discovery set correlated strongly with survival in the validation set (FIGS. 3C and 3D, p<0.0001) and was more efficient at discriminating outcome than mutational load (FIGS. 2D, 2B, 2E, 2H). We analyzed an independent cohort of melanoma patients treated with ipilimumab (n=15) for which we had tissue and matching blood and the signature was validated in this independent set (FIG. 3D).

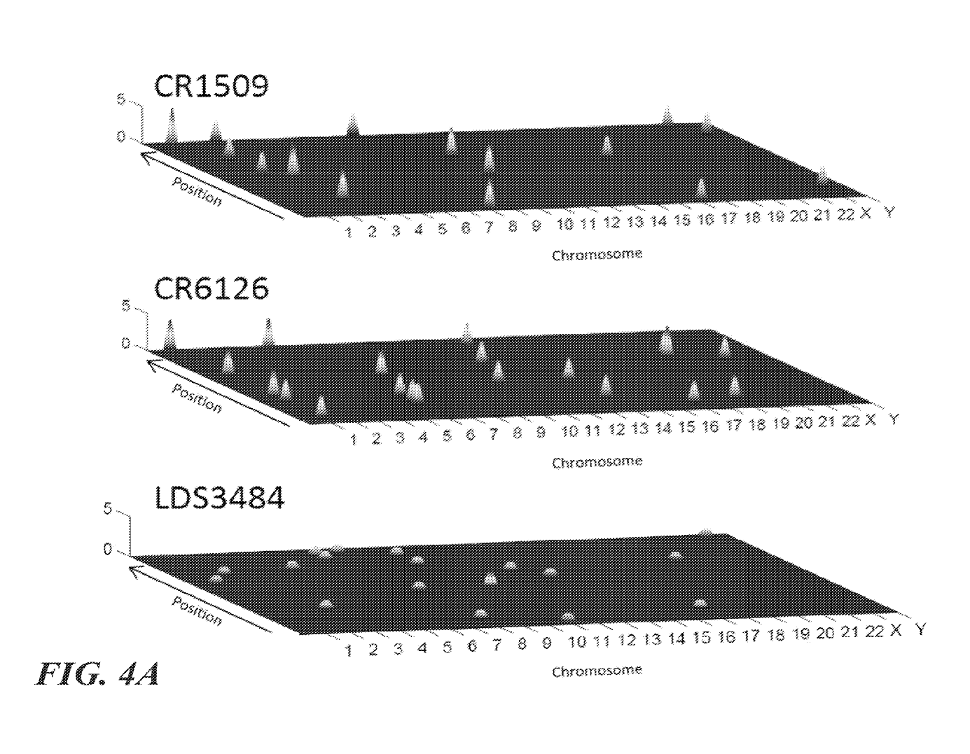

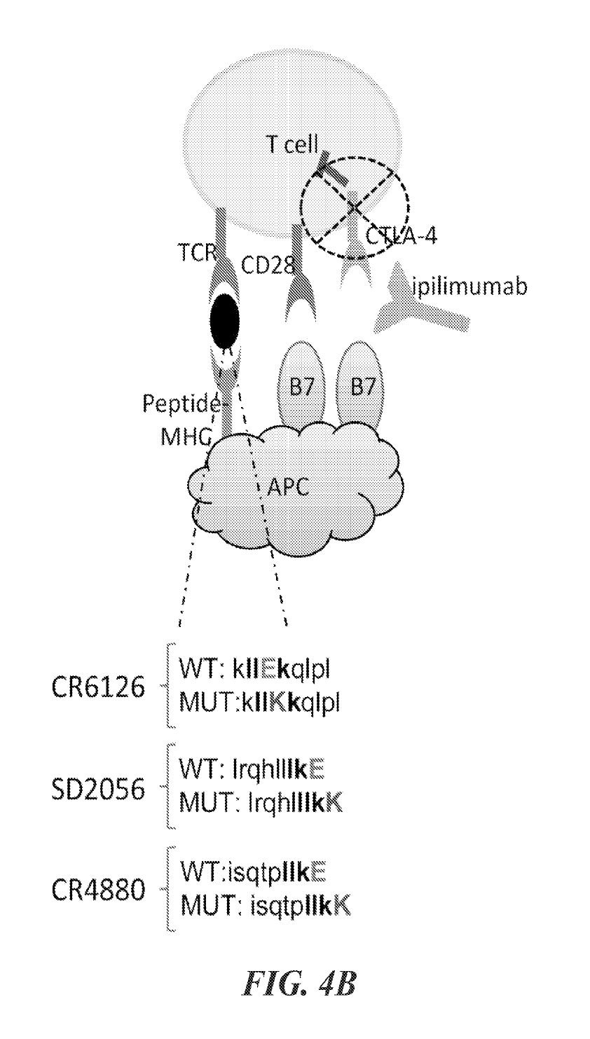

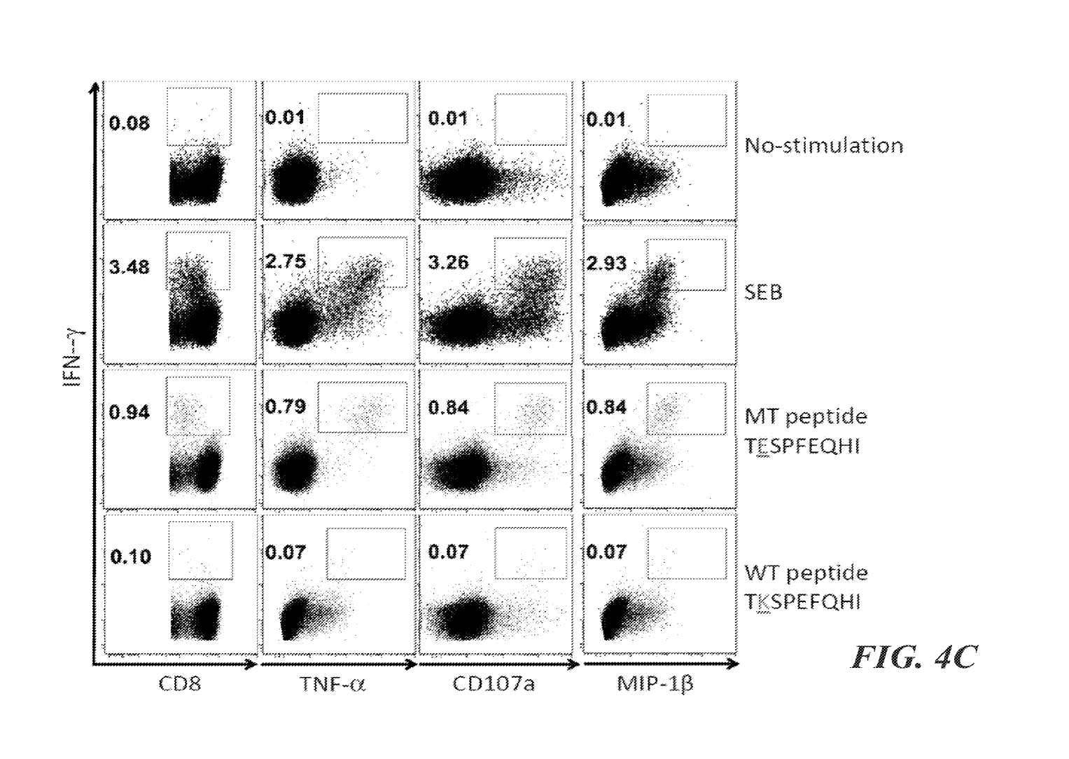

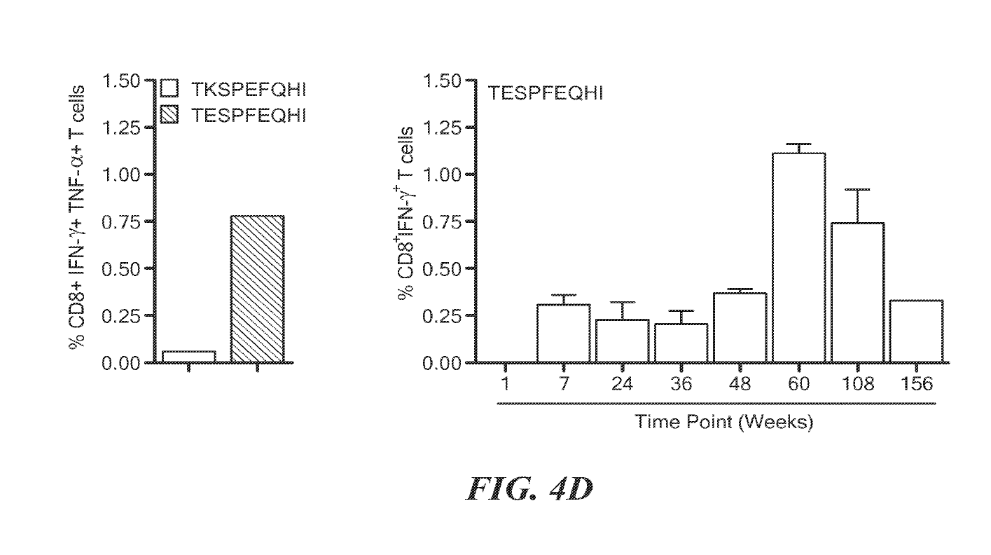

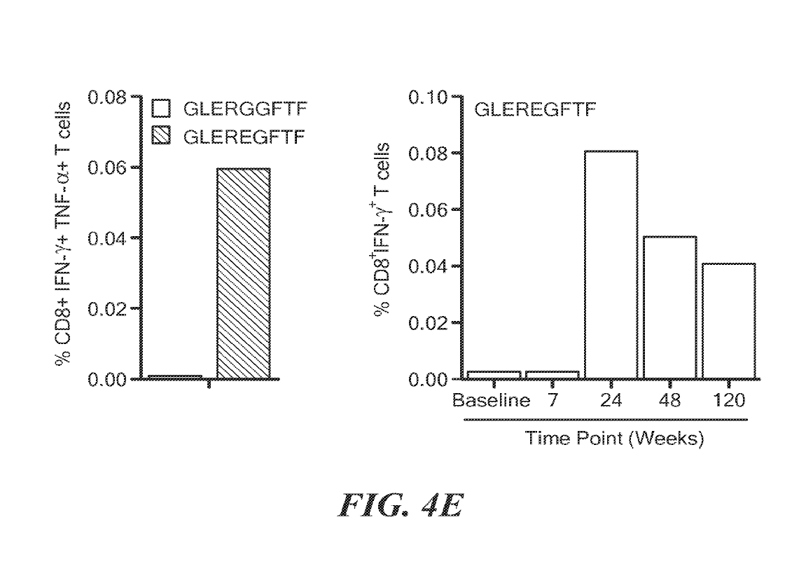

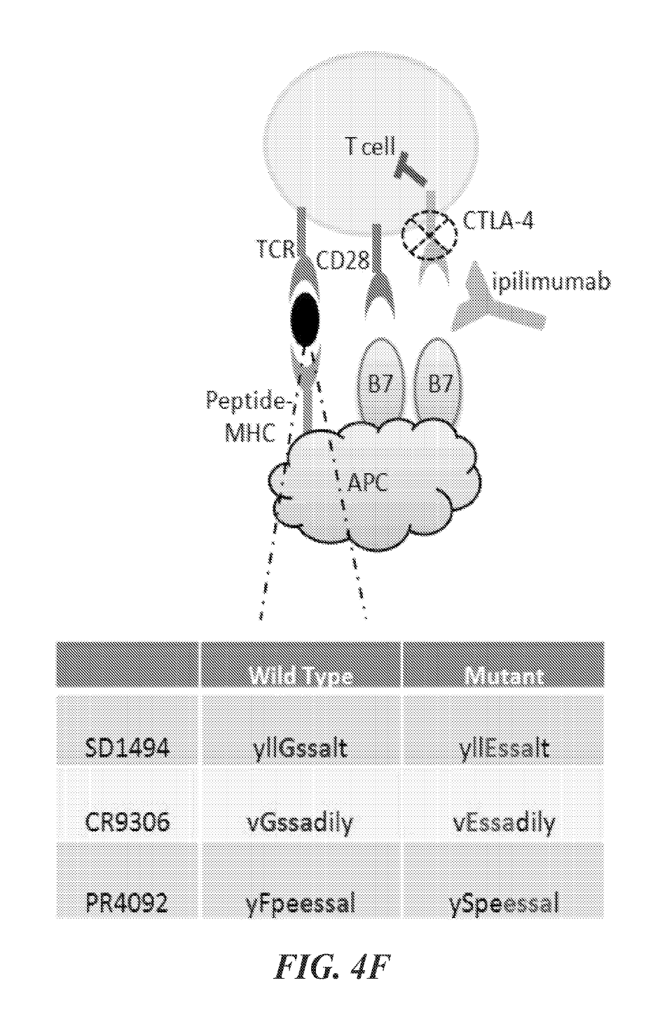

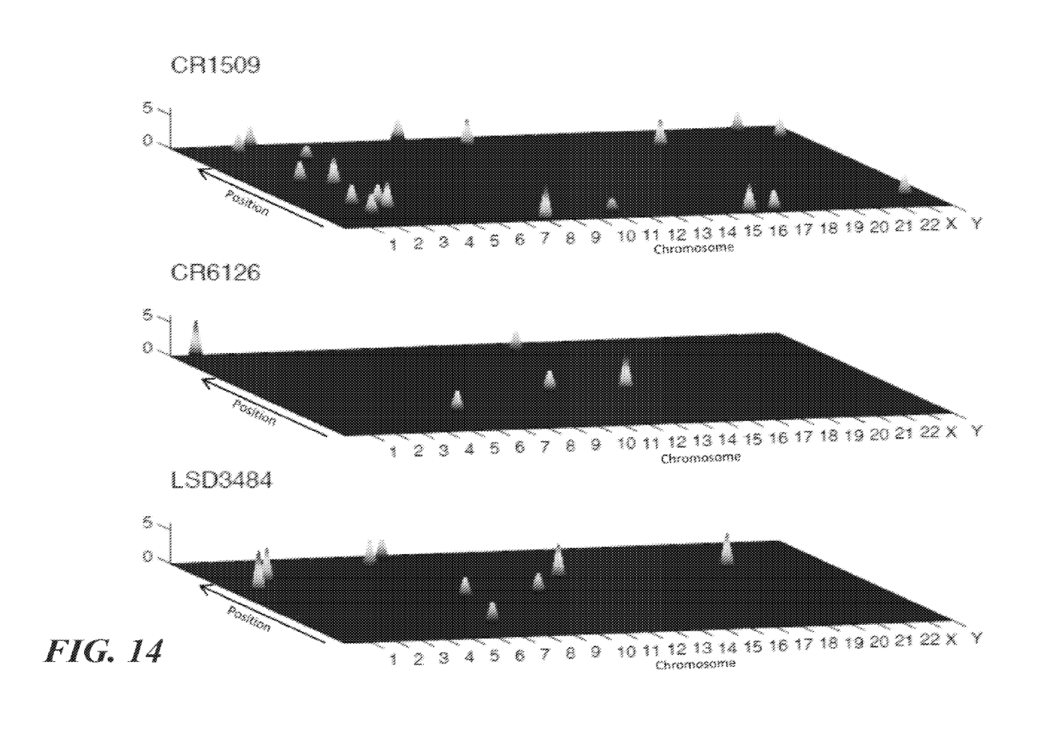

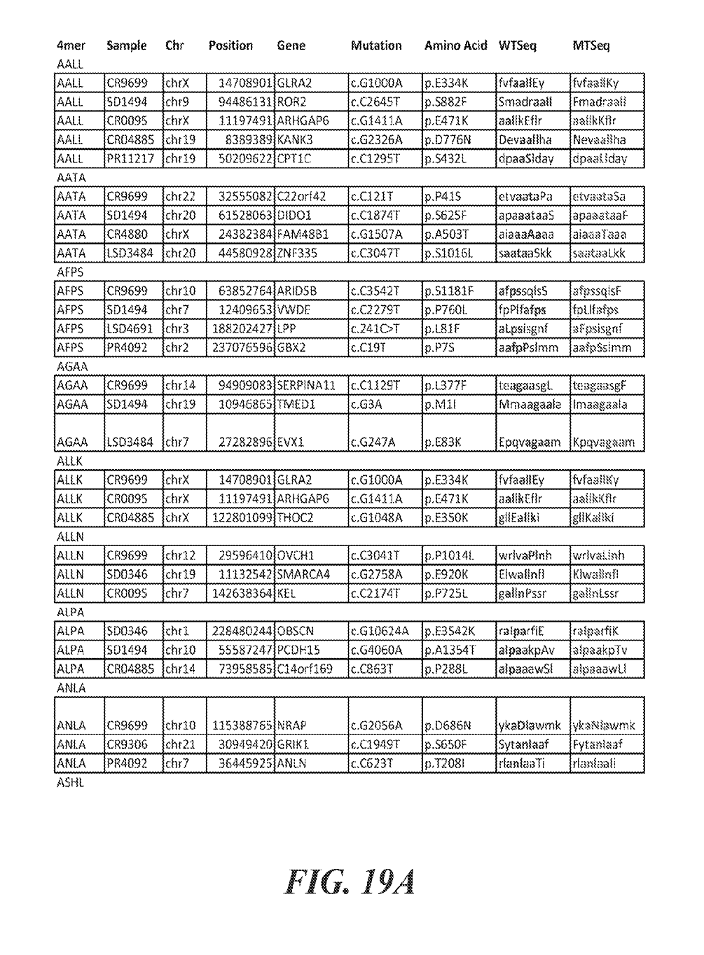

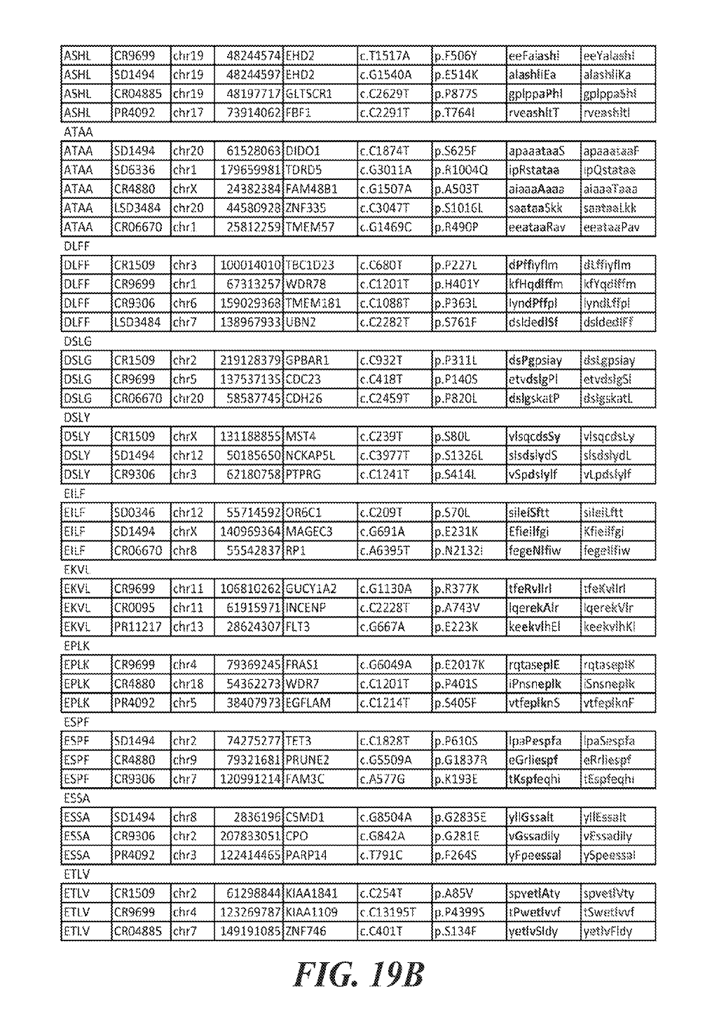

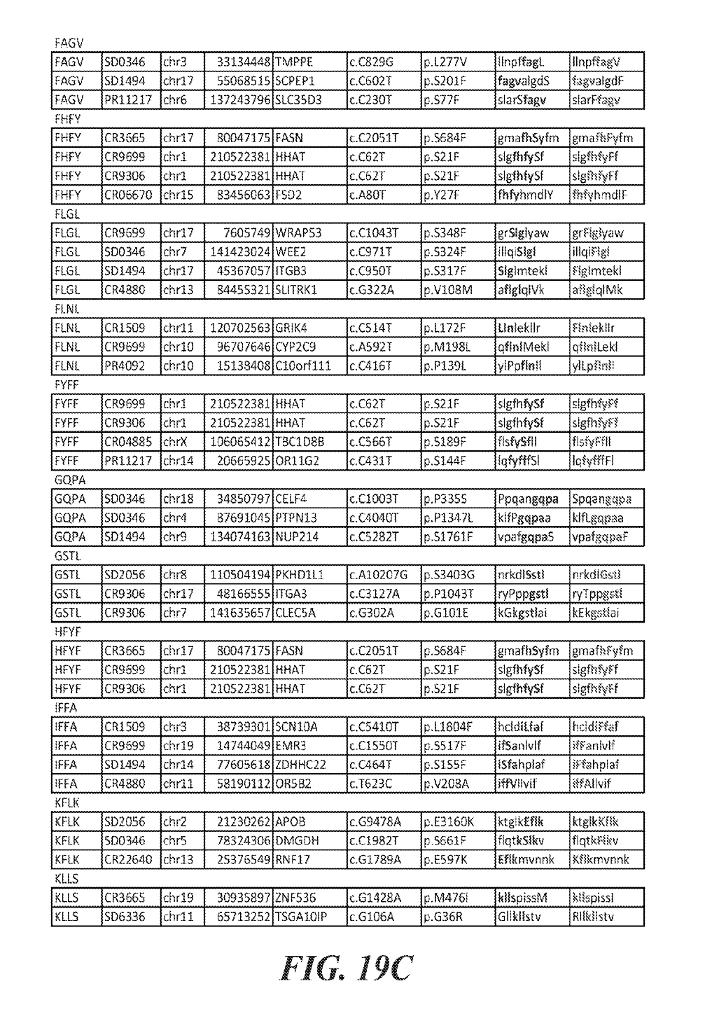

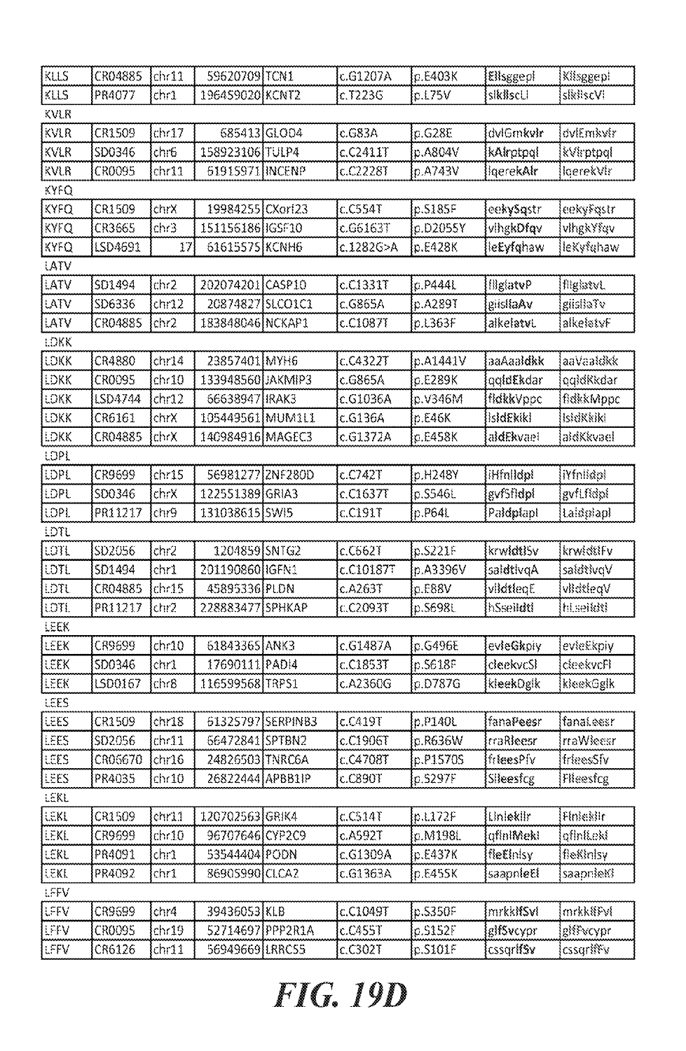

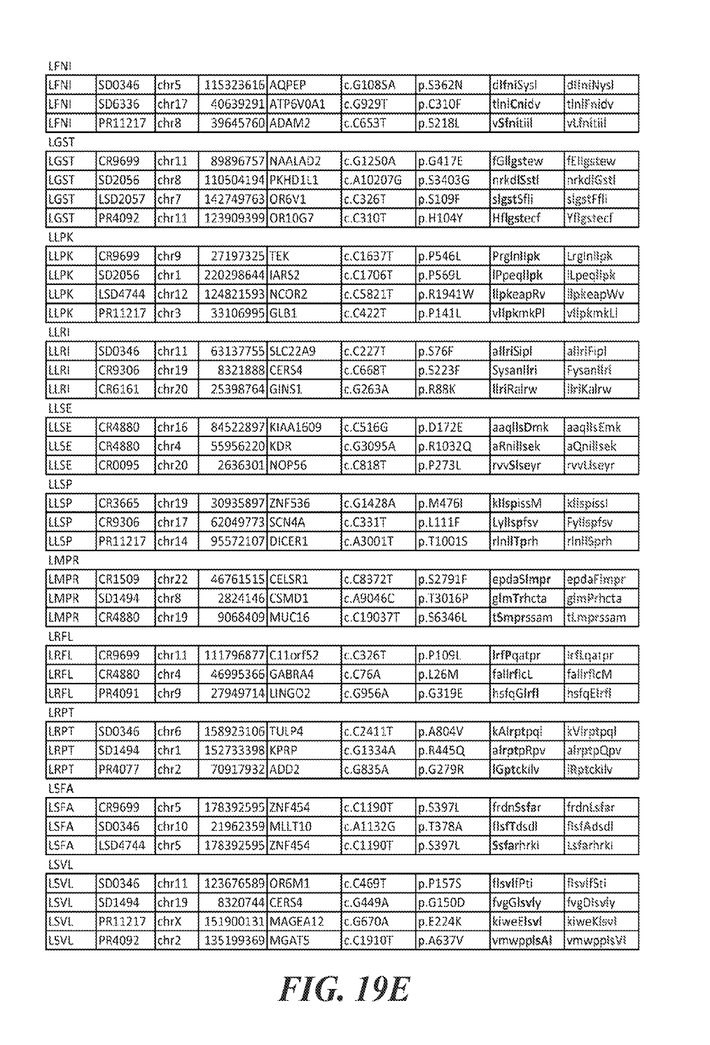

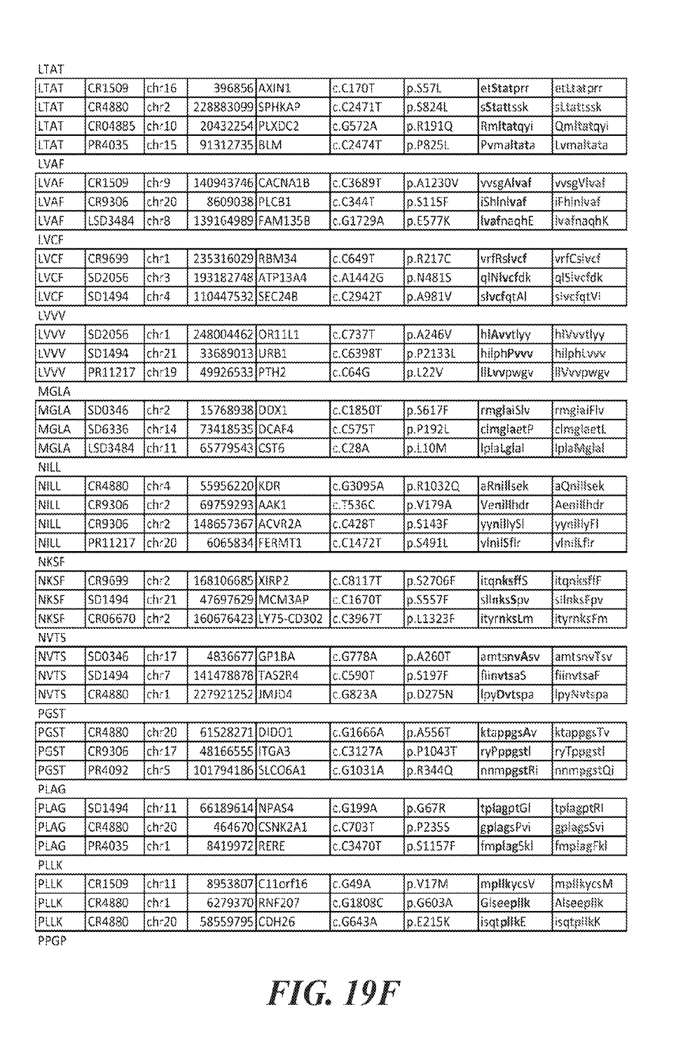

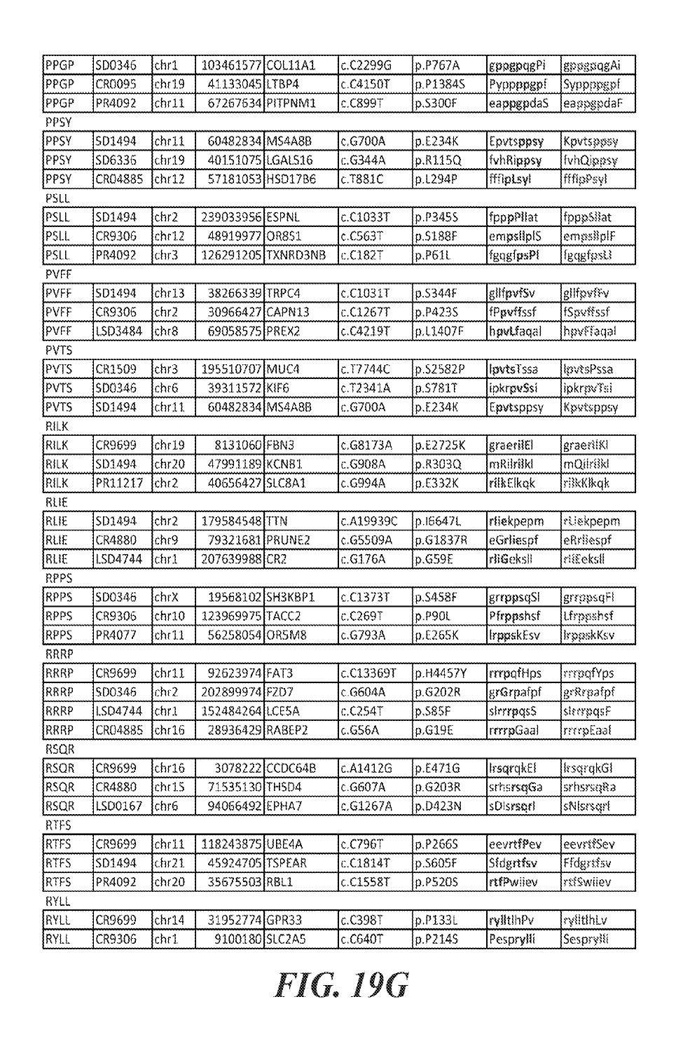

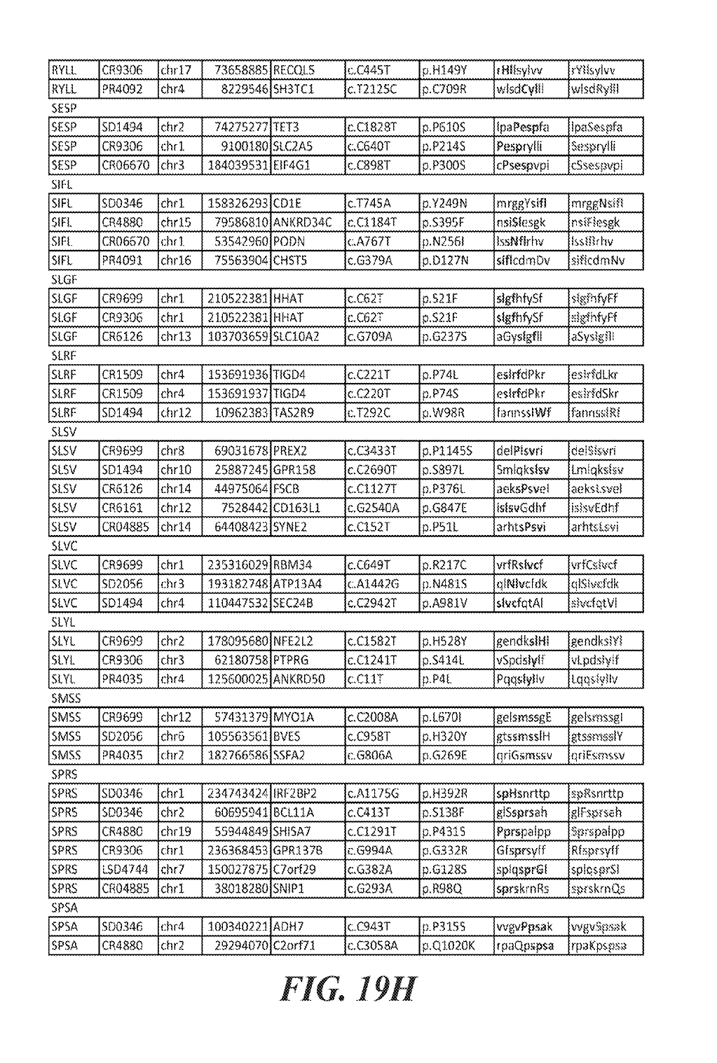

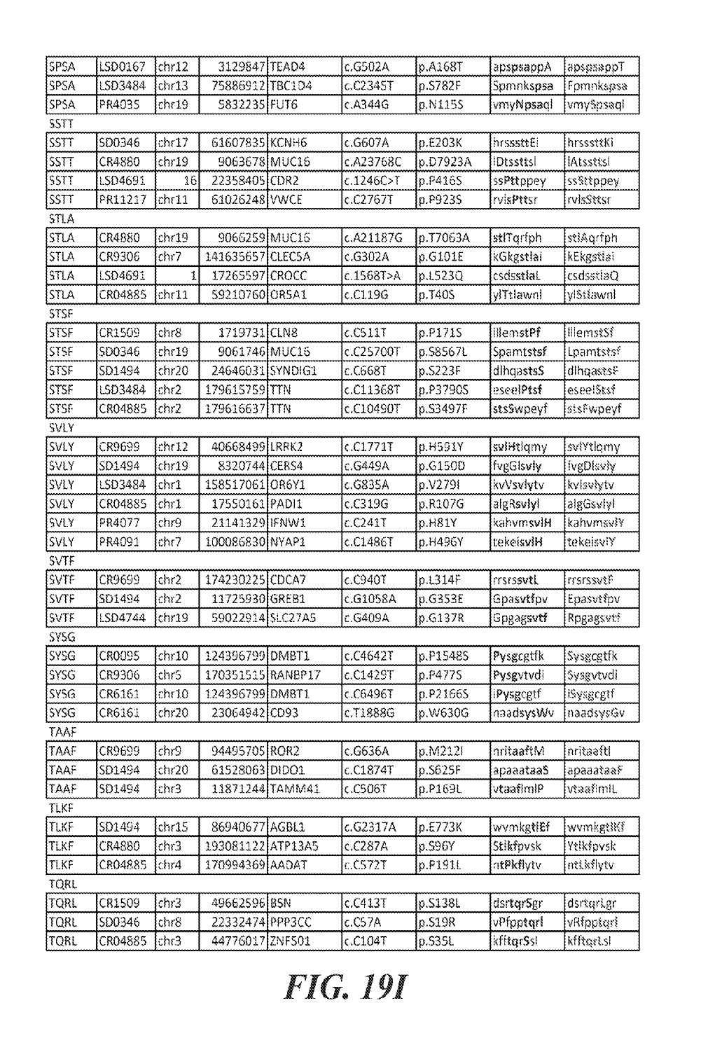

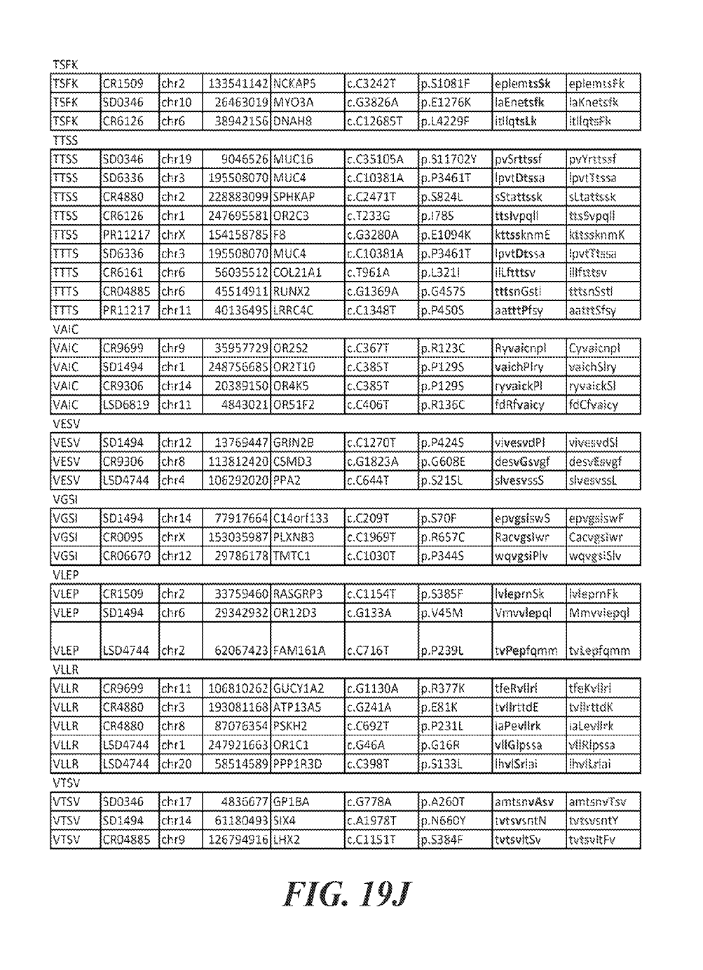

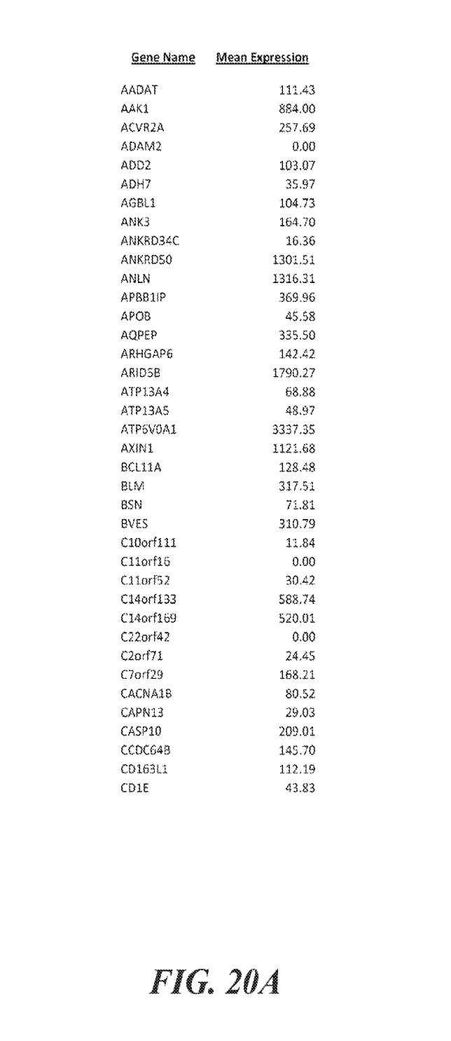

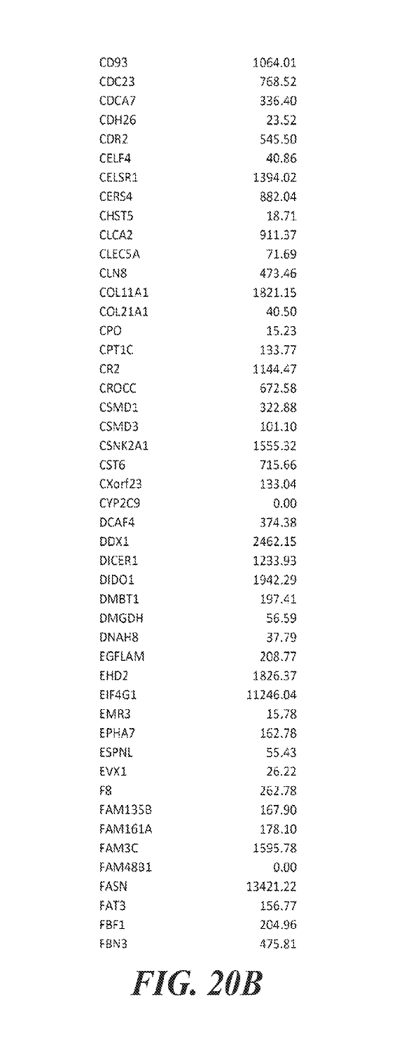

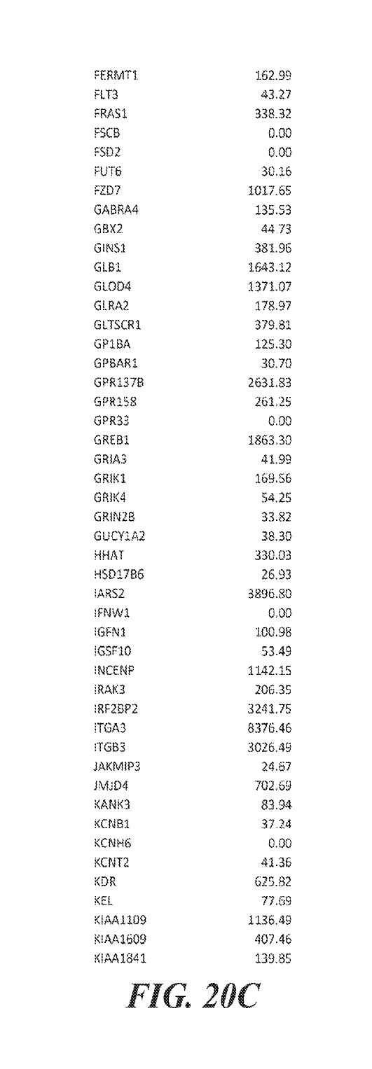

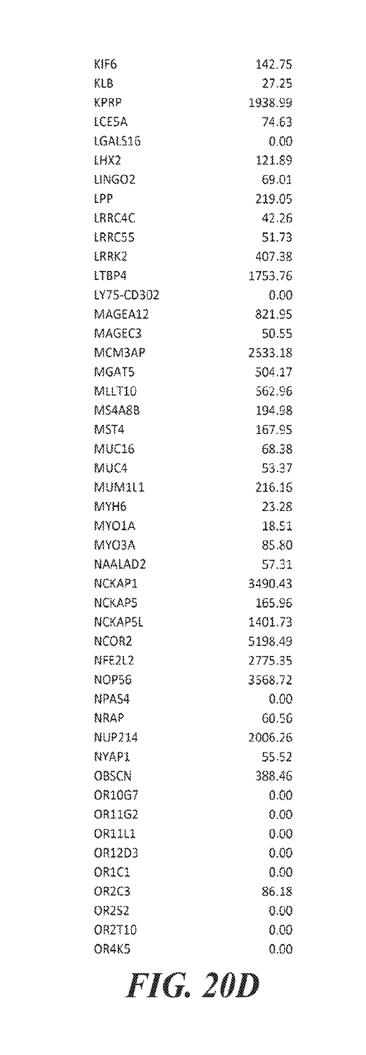

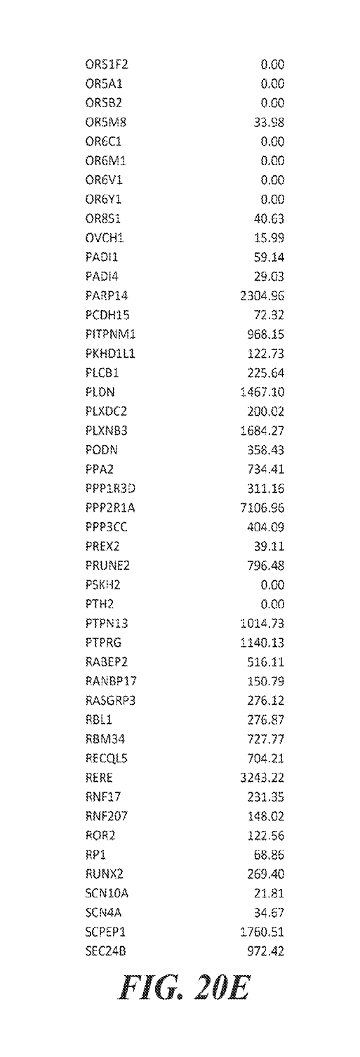

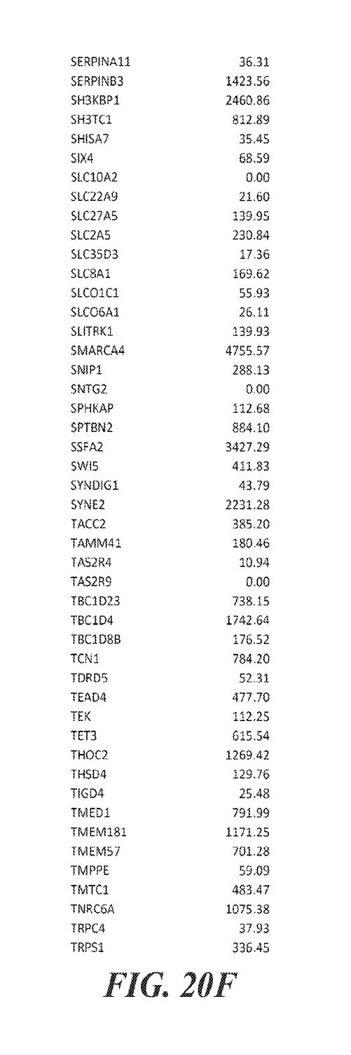

These tetrapeptides were encoded by mutations in diverse genes across the genome (FIG. 4A, FIG. 14, FIG. 19, and Table 4). Using RNASeq data from The Cancer Genome Atlas (TCGA) we confirmed that the genes harboring our somatic neoepitopes were widely expressed in melanoma. In some cases, the amino acid change resulting from the somatic mutation led to a change in the tetrapeptide itself. In others, the mutant amino acid was separate from the tetrapeptide and altered MHC binding, as has been described.^{38, 40, 44-46}

In addition, candidate neoepitopes common to each clinical group were analyzed using the Immune Epitope Database (IEDB). This is the most comprehensive database of experimentally validated, published, and curated antigens and has been used to develop algorithms to identify antigens with high accuracy.²³ We found that the candidate neoepitopes common to benefiters corresponded to many more viral and bacterial antigens in IEDB than the other clinical groups (FIG. 10E, FIG. 10K).

TABLE 4
Context, Genes and Loci for Tetrapeptides in the Response Signature
4mer = common tetrapeptide amino acid sequence. Mut = location
of mutation. WTSeq = predicted wild type 9 amino acid peptide.
MTSeq = predicted mutant 9 amino acid peptide.

| 4mer | Sample | Gene | Mut | WTSeq | MTSeq | Chr | Pos |
|---|---|---|---|---|---|---|---|
| AATA | CR4880 | FAM48B1 | c.G1507A | aiaaaAaaa | aiaaaTaaa | chrX | 24382384 |
| AATA | CR9699 | C22orf42 | c.C121T | etvaataPa | etvaataSa | chr22 | 32555082 |
| AATA | LSD3484 | ZNF335 | c.C3047T | saataaSkk | saataaLkk | chr20 | 44580928 |
| AATA | SD1494 | DIDO1 | c.C1874T | apaaataaS | apaaataaF | chr20 | 61528063 |
| AFPS | LSD4691 | LPP | c.241C > T | aLpsisgnf | aFpsisgnf | chr3 | 188202427 |
| AFPS | CR9699 | ARID5B | c.C3542T | afpssqlsS | afpssqlsF | chr10 | 63852764 |
| AFPS | SD1494 | VWDE | c.C2279T | fpPlfafps | fpLlfafps | chr7 | 12409653 |
| ATAA | CR4880 | FAM48B1 | c.G1507A | aiaaaAaaa | aiaaaTaaa | chrX | 24382384 |
| ATAA | LSD3484 | ZNF335 | c.C3047T | saataaSkk | saataaLkk | chr20 | 44580928 |
| ATAA | SD1494 | DIDO1 | c.C1874T | apaaataaS | apaaataaF | chr20 | 61528063 |
| ATAA | SD6336 | TDRD5 | C.G3011A | ipRstataa | ipQstataa | chr1 | 179659981 |
| DLFF | CR1509 | TBC1D23 | c.C680T | dPffiyflm | dLffiyflm | chr3 | 100014010 |
| DLFF | LSD3484 | UBN2 | c.C2282T | dsldedlSf | dsldedlFf | chr7 | 138967933 |
| DLFF | CR9306 | TMEM181 | c.C1088T | lyndPffpl | lyndLffpl | chr6 | 159029368 |
| DLFF | CR9699 | WDR78 | c.C1201T | kfHqdlffm | kfYqdlffm | chr1 | 67313257 |
| DSAS | SD1494 | UBQLN3 | c.A1358G | glgdsaNrv | glgdsaSrv | chr11 | 5529431 |
| DSAS | CR4880 | FAT1 | c.A4985G | tiadNaspk | tiadSaspk | chr4 | 187542755 |
| DSAS | LSD3484 | CNTNAP2 | c.C3730T | dsasadfPy | dsasadfSy | chr7 | 148106497 |
| DSAS | LSD4744 | KIAA1244 | c.C872T | eSdsaspgv | eLdsaspgv | chr6 | 138576674 |
| ESPF | CR9306 | FAM3C | c.A577G | tKspfeqhi | tEspfeqhi | chr7 | 120991214 |
| ESPF | SD1494 | TET3 | c.C1828T | lpaPespfa | lpaSespfa | chr2 | 74275277 |
| ESPF | CR4880 | PRUNE2 | c.G5509A | eGrliespf | eRrliespf | chr9 | 79321681 |
| ESSF | LSD0167 | EGF | c.C1880T | npriessSl | npriessFl | chr4 | 110897218 |
| ESSF | CR9306 | KIR2DL4 | c.C691T | tePsfktgi | teSsfktgi | chr19 | 55320323 |
| ESSF | SD1494 | RLF | c.C5297T | Pmgfessfl | Lmgfessfl | chr1 | 40705671 |
| FFYV | CR9699 | AP2M1 | c.C169T | artsffHvk | artsffYvk | chr3 | 183896739 |
| FFYV | LSD0167 | OR9A4 | c.C772T | clfLyvkpk | clffyvkpk | chr7 | 141619447 |
| FFYV | SD1494 | GJB5 | c.C436T | svdiafLyv | svdiaffyv | chr1 | 35223367 |
| FLGL | CR9699 | WRAP53 | c.C1043T | grSlglyaw | grFlglyaw | chr17 | 7605749 |
| FLGL | SD0346 | WEE2 | c.C971T | illqiSlgl | illqiFlgl | chr7 | 141423024 |
| FLGL | SD1494 | ITGB3 | c.C950T | Slglmtekl | Flglmtekl | chr17 | 45367057 |
| FLGL | CR4880 | SLITRK1 | c.G322A | aflglqlVk | aflglqlMk | chr13 | 84455321 |
| FPGP | CR9699 | CCBE1 | c.C733T | tyLpgppgl | tyFpgppgl | chr18 | 57115257 |
| FPGP | CR6126 | WDR46 | c.C289T | dPfpgpapv | dSfpgpapv | chr6 | 33256462 |
| FPGP | LSD3484 | MSR1 | c.C902T | fpgpigPpg | fpgpigLpg | chr8 | 16007817 |
| IFFA | CR1509 | SCN10A | c.C5410T | hcldiLfaf | hcldiFfaf | chr3 | 38739301 |
| IFFA | CR9699 | EMR3 | c.C1550T | ifSanlvlf | ifFanlvlf | chr19 | 14744049 |
| IFFA | SD1494 | ZDHHC22 | c.C464T | iSfahplaf | iFfahplaf | chr14 | 77605618 |
| IFFA | CR4880 | OR5B2 | c.T623C | iffVllvif | iffAllvif | chr11 | 58190112 |
| KLLK | CR6126 | SPTA1 | c.G6097A | kllEkqlpl | kllKkqlpl | chr1 | 158592796 |
| KLLK | SD1494 | CEACAM3 | c.G694A | Ellkhdtni | Kllkhdtni | chr19 | 42315210 |
| KLLK | SD0346 | LRRIQ3 | c.G811A | kllkDlffk | kllkNlffk | chr1 | 74575134 |
| KTPF | CR9699 | EYS | c.G4169A | rraRtpfim | rraKtpfim | chr6 | 65301591 |
| KTPF | SD0346 | HMHA1 | c.C41T | lmktpSisk | lmktpFisk | chr19 | 1067445 |
| KTPF | CR6126 | SLC13A5 | c.C1180T | ktpfypPpl | ktpfypSpl | chr17 | 6596458 |
| KYFQ | CR1509 | CXorf23 | c.C554T | eekySqstr | eekyFqstr | chrX | 19984255 |
| KYFQ | CR3665 | IGSF10 | c.G6163T | vlhgkDfqv | vlhgkYfqv | chr3 | 151156186 |
| KYFQ | LSD4691 | KCNH6 | c.1282G > A | leEyfqhaw | laKyfqhaw | chr17 | 61615575 |
| LAIF | CR6126 | JPH2 | c.C2068T | laiLfvhll | laiFfvhll | chr20 | 42743459 |
| LAIF | LSD3484 | OR51S1 | c.C470T | kislaiSfr | kislaiFfr | chr11 | 4869969 |
| LAIF | LSD4744 | KRIT1 | c.C1585T | iedplaiLi | iedplaiFl | chr7 | 91844070 |
| LAIF | SD0346 | DDX1 | c.C1850T | rmglaiSlv | rmglaiFlv | chr2 | 15768938 |
| LATL | LSD0167 | PIGO | c.C1630T | rplatlfSi | rplatlfSi | chr9 | 35092254 |
| LATL | L5D3484 | EIF3A | c.C967T | Llatlsipi | Flatlsipi | chr10 | 120825066 |
| LATL | LSD6336 | EVC | c.C1964T | nAlatltqm | nVlatltqm | chr4 | 5798826 |
| LEEK | CR9699 | ANK3 | c.G1487A | evleGkpiy | evleEkply | chr10 | 61843365 |
| LEEK | LSD0167 | TRPS1 | c.A2360G | kleekDglk | kleekGglk | chr8 | 116599568 |
| LEEK | SD0346 | PADI4 | c.C1853T | cleekvcSl | cleekvcFl | chr1 | 17690111 |
| LFFV | CR6126 | LRRC55 | c.C302T | cssqrlfSv | cssqrlfFv | chr11 | 56949669 |
| LFFV | CR9699 | KLB | c.C1049T | mrkklfSvl | mrkklfFvl | chr4 | 39436053 |
| LFFV | CR0095 | PPP2R1A | c.C455T | glfSvcypr | glfFvcypr | chr19 | 52714697 |
| LLKK | CR6126 | SPTA1 | c.G6097A | kllEkqlpl | kllKkqlpl | chr1 | 158592796 |
| LLKK | SD2056 | FANCB | c.G1246A | lrqhlllkE | lrqhlllkK | chrX | 14871241 |
| LLKK | CR4880 | CDH26 | c.G643A | isqtpllkE | isqtpllkK | chr20 | 58559795 |
| LLKK | CR0095 | ARHGAP6 | c.G1411A | aallkEflr | aallkKflr | chrX | 11197491 |
| LPLA | LSD3484 | CST6 | c.C28A | lplaLglal | lplaMglal | chr11 | 65779543 |
| LPLA | SD2056 | ACSL6 | c.C986T | Sflplahmf | Fflplahmf | chr5 | 131310626 |
| LPLA | SD6336 | ALAD | c.C836T | lplavyhvS | lplavyhvF | chr9 | 116151352 |
| LSRS | CR6126 | SRSF11 | c.C1109T | klsrspSpr | klsrspFpr | chr1 | 70715721 |
| LSRS | LSD0167 | EPHA7 | c.G1267A | sDlsrsqrl | sNlsrsqrl | chr6 | 94066492 |
| LSRS | LSD3484 | MYH3 | c.A3899T | ivSqlsrsk | ivLqlsrsk | chr17 | 10539128 |
| LSSV | CR9699 | ZFHX4 | c.C1532T | plSssvlkf | plLssvlkf | chr8 | 77617855 |
| LSSV | LSD4744 | AMBN | c.C1126T | glPsvtpaa | glSsvtpaa | chr4 | 71472229 |
| LSSV | CR1509 | FAT3 | c.C3074T | rpvslssvS | rpvslssvF | chr11 | 92088352 |
| LSSV | CR6126 | C7orf63 | c.T1271C | halatlssv | haTatlssv | chr7 | 89909106 |
| LVAF | CR1509 | CACNA1B | c.C3689T | vvsgAlvaf | vvsgVlvaf | chr9 | 140943746 |
| LVAF | LSD3484 | FAM135B | c.G1729A | lvafnaqhE | lvafnaqhK | chr8 | 139164989 |
| LVAF | CR9306 | PLCB1 | c.C344T | iShlnlvaf | iFhlnlvaf | chr20 | 8609038 |
| LVAL | CR9699 | OVCH1 | c.C3041T | wrlvaPlnh | wrlvaLlnh | chr12 | 29596410 |
| LVAL | LSD3484 | MNAT1 | c.C578T | ssdlPvall | ssdiLvall | chr14 | 61285456 |
| LVAL | CR3665 | MAP4K1 | c.G119A | kvsGdlval | kvsEdlval | chr19 | 39108246 |
| LVAL | SD0346 | ABCA12 | c.T5954A | slldilval | slNdilval | chr2 | 215823164 |
| MGLA | LSD3484 | CST6 | c.C28A | lplaLglal | lplaMglal | chr11 | 65779543 |
| MGLA | SD0346 | DDX1 | c.C1850T | rmglaiSlv | rmglaiFlv | chr2 | 15768938 |
| MGLA | SD6336 | DCAF4 | c.C575T | clmglaetP | clmglaetL | chr14 | 73418535 |
| PVFF | LSD3484 | PREX2 | c.C4219T | hpvLfaqal | hpvFfaqal | chr8 | 69058575 |
| PVFF | SD1494 | TRPC4 | c.C1031T | gllfpvfSv | gllfpvfSv | chr13 | 38266339 |
| PVFF | CR9306 | CAPN13 | c.C1267T | fPpvffssf | fSpvffssf | chr2 | 30966427 |
| QKGV | CR6126 | SEMG2 | c.G1270A | gekDvqkgv | gekNvqkgv | chr20 | 43851543 |
| QKGV | LSD3484 | FAM116A | c.A1272C | qlqkgvQqk | qlqkgvHgk | chr3 | 57619073 |
| QKGV | CR4880 | SELRC1 | c.A656G | lhKeqqkgv | lhReqqkgv | chr1 | 53153432 |
| RSQR | CR4880 | THSD4 | c.G607A | srhsrsqGa | srhsrsqRa | chr15 | 71535130 |
| RSQR | CR9699 | CCDC64B | c.A1412G | lrsqrqkEl | lrsqrqkGl | chr16 | 3078222 |
| RSQR | LSD0167 | EPHA7 | c.G1267A | sDlsrsqrl | sNlsrsqrl | chr6 | 94066492 |
| SAPS | CR9306 | ATP10D | c.C3478T | lftsapPvi | lftsapSvi | chr4 | 47578901 |
| SAPS | CR1509 | RYR2 | c.C2300G | lsapsiSfr | lsapsiWfr | chr1 | 237664107 |
| SAPS | LSD0167 | DOCK3 | c.G5265A | thsapsqMi | thsapsqli | chr3 | 51400077 |
| SAPS | SD0346 | CTBP2 | c.C1217T | rPssapsqh | rLssapsqh | chr10 | 126715112 |
| SAPS | SD1494 | T | c.C1184T | hpvsapsSs | hpvsapsFs | chr6 | 166571927 |
| SDSY | LSD4691 | SRRT | c.271C > T | ssdPyhsgy | ssdSyhsgy | chr7 | 100479299 |
| SDSY | CR4880 | UNC13D | c.C400T | fsdPycllg | fsdSycllg | chr17 | 73838683 |
| SDSY | CR9699 | TBC1D8 | c.G952A | Grmfasdsy | Rrmfasdsy | chr2 | 101656723 |
| SLGF | CR6126 | SLC10A2 | c.G709A | aGyslgfll | aSyslgfll | chr13 | 103703659 |
| SLGF | CR9699 | HHAT | c.C62T | slgfhfySf | slgfhfyFf | chr1 | 210522381 |
| SLGF | CR9306 | HHAT | c.C62T | slgfhfySf | slgfhfyFf | chr1 | 210522381 |
| SLSV | CR6126 | FSCB | c.C1127T | aeksPsvel | aeksLsvel | chr14 | 44975064 |
| SLSV | CR9699 | PREX2 | c.C3433T | delSlsvri | delSlsvri | chr8 | 69031678 |
| SLSV | SD1494 | GPR158 | c.C2690T | Smlqkslsv | Lmlqkslsv | chr10 | 25887245 |
| SPLY | CR1509 | NEUROD1 | c.C689T | lpspPygtm | lpspLygtm | chr2 | 182542899 |
| SPLY | CR6126 | OR4L1 | c.C158T | rStlhsply | rLtlhsply | chr14 | 20528361 |
| SPLY | SD1494 | ANGEL1 | c.C1312T | nsvPdsply | nsvSdsply | chr14 | 77272827 |
| SPRS | LSD4744 | C7orf29 | c.G382A | splqsprGl | splqsprSl | chr7 | 150027875 |
| SPRS | SD0346 | IRF2BP2 | c.A1175G | spHsnrttp | spRsnrttp | chr1 | 234743424 |
| SPRS | CR4880 | SHISA7 | c.C1291T | Pprspalpp | Sprspalpp | chr19 | 55944849 |
| SPRS | SD0346 | BCL11A | c.C413T | glSsprsah | glFsprsah | chr2 | 60695941 |
| SPRS | CR9306 | GPR137B | c.G994A | Gfsprsyff | Rfsprsyff | chr1 | 236368453 |
| SPSA | SD0346 | ADH7 | c.C943T | vvgvPpsak | vvgvSpsak | chr4 | 100340221 |
| SPSA | LSD0167 | TEAD4 | c.G502A | apspsappA | apspsappT | chr12 | 3129847 |
| SPSA | LSD3484 | TBC1D4 | c.C2345T | Spmnkspsa | Fpmnkspsa | chr13 | 75886912 |
| SPSA | CR4880 | C2orf71 | c.C3058A | rpaQpspsa | rpaKpspsa | chr2 | 29294070 |
| SRLK | SD2056 | PCDHGA4 | c.G2266A | rrwhksrlL | rrwhksrlK | chr5 | 140737033 |
| SRLK | CR4880 | LRRC37B | c.C448T | alvqlPrlk | alvqlSrlk | chr17 | 30348613 |
| SRLK | CR6126 | MCM3 | c.T2375A | esrlkaFkv | esrlkaKkv | chr6 | 52129438 |
| SRSQ | CR9306 | PTK6 | c.G1150A | hemfsrGqv | hemfsrSqv | chr20 | 62161449 |
| SRSQ | LSD0167 | EPHA7 | c.G1267A | sDlsrsqrl | sNlsrsqrl | chr6 | 94066492 |
| SRSQ | CR4880 | THSD4 | c.G607A | srhsrsqGa | srhsrsqRa | chr15 | 71535130 |
| SRSQ | CR4880 | BCLAF1 | c.C56T | srsksrsqS | srsksrsqF | chr6 | 136600949 |
| SSPL | CR6126 | CLCNKA | c.C1130T | mtqnsspP | mtqnsspL | chr1 | 16355697 |
| | | | | w | w | | |
| SSPL | CR4880 | LINS | c.G2040T | sleppsRpl | sleppsSpl | chr15 | 101109677 |
| SSPL | LSD3484 | C10orf26 | c.C521T | Sqgaqsspl | Fqgaqsspl | chr10 | 104572517 |
| SSTL | SD1494 | OR10K2 | c.C685T | ailqfPstl | ailqfSstl | chr1 | 158389972 |
| SSTL | LSD4691 | CROCC | c.1568T > A | csdsstlaL | csdsstlaQ | chr1 | 17265597 |
| SSTL | CR0095 | MUC16 | c.C27467T | sSspvsstl | sFspvsstl | chr19 | 9059979 |
| SSTT | LSD4691 | CDR2 | c.1246C > T | ssPttppey | ssSttppey | chr16 | 22358405 |
| SSTT | SD0346 | KCNH6 | c.G607A | hrsssttEl | hrsssttKl | chr17 | 61607835 |
| SSTT | CR4880 | MUC16 | c.A23768C | lDtssttsl | lAtssttsl | chr19 | 9063678 |
| STLA | CR4880 | MUC16 | c.A21187G | stlTqrfph | stlAqrfph | chr19 | 9066259 |
| STLA | LSD4691 | CROCC | c.1568T > A | csdsstlaL | csdsstlaQ | chr1 | 17265597 |
| STLA | CR9306 | CLEC5A | c.G302A | kGkgstlai | kEkgstlai | chr7 | 141635657 |
| STSF | CR1509 | CLN8 | c.C511T | lllemstPf | lllemstSf | chr8 | 1719731 |
| STSF | LSD3484 | TTN | c.C11368T | eseelPtsf | eseelStsf | chr2 | 179615759 |
| STSF | SD1494 | SYNDIG1 | c.C668T | dlhqastsS | dlhqastsF | chr20 | 24646031 |
| STSF | SD0346 | MUC16 | c.C25700T | Spamtstsf | Lpamtstsf | chr19 | 9061746 |
| SVLY | CR9699 | LRRK2 | c.C1771T | svlHtlqmy | svlYtlqmy | chr12 | 40668499 |
| SVLY | LSD3484 | OR6Y1 | c.G835A | kvVsvlytv | kvlsvlytv | chr1 | 158517061 |
| SVLY | SD1494 | CERS4 | c.G449A | fvgGlsvly | fvgDlsvly | chr19 | 8320744 |
| TKSF | CR6126 | KITLG | c.C544T | vsvtkPfml | vsvtkSfml | chr12 | 88909371 |
| TKSF | CR9306 | RGR | c.T539A | lftMsffnf | lftKsffnf | chr10 | 86014108 |
| TKSF | LSD4691 | IL18R1 | c.446G > A | tGgtdtksf | tEgtdtksf | chr2 | 103003422 |
| TLAQ | LSD4691 | CROCC | c.1568T > A | csdsstlaL | csdsstlaQ | chr1 | 17265597 |
| TLAQ | CR4880 | MUC16 | c.A21187G | stlTqrfph | stlAqrfph | chr19 | 9066259 |
| TLAQ | CR6126 | GATSL3 | c.C403T | viHtlaqef | viYtlaqef | chr22 | 30683246 |
| TQSA | LSD0167 | RNPEPL1 | c.G707A | lmsatRsay | lmsatQsay | chr2 | 241512564 |
| TQSA | LSD4691 | SDK1 | c.6559A > C | Ttqsaggvy | Ptqsaggvy | chr7 | 4304933 |
| TQSA | CR4880 | ZNF536 | c.C2378T | gtqsaSlky | gtqsaFlky | chr19 | 31038904 |
| TSFK | CR1509 | NCKAP5 | c.C3242T | eplemtsSk | eplemtsFk | chr2 | 133541142 |
| TSFK | CR6126 | DNAH8 | c.C12685T | itllqtsLk | itllqrsFk | chr6 | 38942156 |
| TSFK | SD0346 | MYO3A | c.G3826A | laEnetsfk | laKnetsfk | chr10 | 26463019 |
| TTSS | CR6126 | OR2C3 | c.T233G | ttslvpqll | ttsSvpqll | chr1 | 247695581 |
| TTSS | SD6336 | MUC4 | c.C10381A | lpvtDtssa | lpvtTtssa | chr3 | 195508070 |
| TTSS | SD0346 | MUC16 | c.C35105A | pvSrttssf | pvYrttssf | chr19 | 9046526 |
| TTSS | CR4880 | SPHKAP | c.C2471T | sStattssk | sLtattssk | chr2 | 228883099 |
| VDSL | CR6126 | GPRIN1 | c.C655T | kvdPlcssk | kvdSlcssk | chr5 | 176026181 |
| VDSL | SD1494 | GRIN2B | c.C1270T | vivesvdPl | vivesvdSl | chr12 | 13769447 |
| VDSL | CR6126 | PKN2 | c.G2092A | Evdslmcek | Kvdslmcek | chr1 | 89273448 |
| VDSL | CR9699 | CDC23 | c.C418T | etvdslgPl | etvdslgSl | chr5 | 137537135 |
| VILS | CR6126 | CLEC4G | c.G136A | vlwAvilsi | vlwTvilsi | chr19 | 7796577 |
| VILS | LSD3484 | PCDHB1 | c.T2099A | vilsFlfll | vilsYlfll | chr5 | 140433154 |
| VILS | SD1494 | TMEM74 | c.A754T | vilscllmM | vilscllmL | chr8 | 109796574 |
| VVLL | CR1509 | ZP1 | c.C41T | ypvAllllv | ypvVllllv | chr11 | 60635075 |
| VVLL | LSD3484 | PRRG3 | c.C254T | yvvvPllgv | yvvvLllgv | chrX | 150869063 |
| VVLL | LSD4744 | ANK3 | c.C518T | ghdqvvSll | ghdqvvLll | chrl0 | 62023723 |
| VVLL | CR0095 | SLC17A4 | c.C491T | gvAllivlr | gvVllivlr | chr6 | 25770488 |
| VVLL | CR0095 | NOP56 | c.C818T | rvvSlseyr | rvvLlseyr | chr20 | 2636301 |
| YPSS | CR1509 | HOXB1 | c.C334T | Hpssygaql | Ypssygaql | chr17 | 46607933 |
| YPSS | CR4880 | POU2F3 | c.C1169A | Rpsspgsgl | Ypsspgsgl | chr11 | 120187971 |
| YPSS | LSD0167 | ATG13 | c.C655T | rPypssspm | rSypssspm | chr11 | 46679132 |

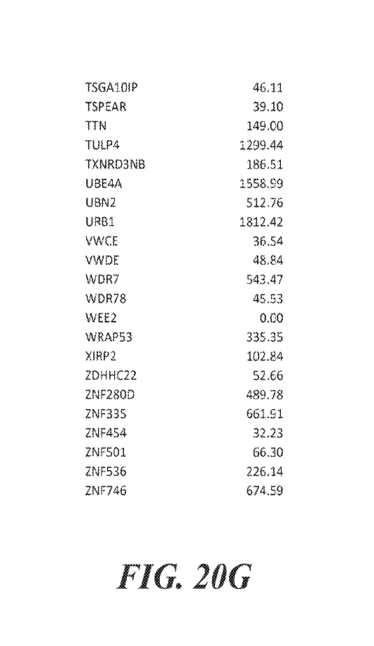

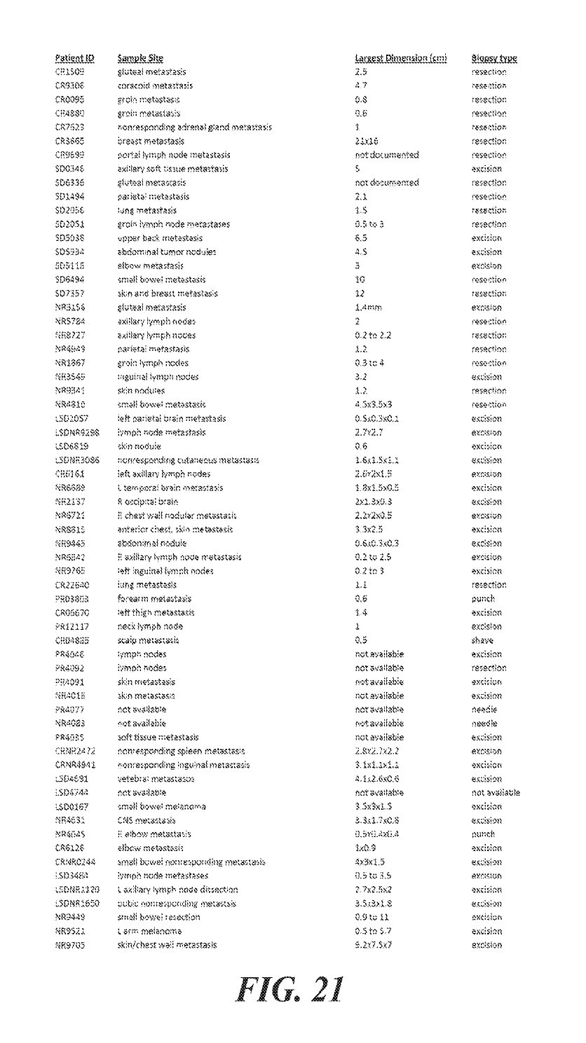

For example, the analysis presented in Table 5 and FIG. 21 demonstrates that a tetrapeptide substring ESSA is shared by patients in the benefitting group (see also FIG. 4F) and corresponds to the human cytomegalovirus immediate earlyt epitope (MESSAKRKMDPDNPD). Additionally, the tetrapeptide substring LLKK may be shared by patients in the LB group; this substring corresponds to the precise antigenic portion of Toxoplasma gondii granule antigen (RSFKDLLKK, FIG. 4B).^{47,48} These data suggest that the neoepitopes in patients with strong clinical benefit from CTLA-4 blockage (e.g., patients with strong responses to ipilimumab and tremelimumab) may resemble epitopes from pathogens which T cells are geared to recognize.

Using a whole exome sequencing approach, we characterized the entire predicted antigenic peptide space (see Methods). As further validation of our study, we “rediscovered” melanoma antigen recognized by T cells (MART-1, also known as MelanA), an experimentally validated melanocytic antigen (FIG. 1 OF).^{37,49-51} EKLS was shared by complete and long-term responders, comprises the core amino acids of the MART-1 MHC Class II epitope, and the phospho-serine moiety is critical to T-cell receptor (TCR) recognition.^{51,52}

TABLE 5
Sample Site, Size and Type

| Patient | | Largest | | Biopsy |
|---|---|---|---|---|
| ID | Sample Site | Dimension | | Type |
| CR1509 | gluteal lesion | 2.5 | cm | resection |
| CR9306 | coracoid lesion | 4.7 | cm | resection |
| CR0095 | groin lesion | 0.8 | cm | resection |
| CR4880 | groin lesion | 0.6 | cm | resection |
| CR7623 | adrenal gland | 1 | cm | resection |
| CR3665 | breast lesion | 21 × 16 | cm | resection |
| CR9699 | portal lymph node | not documented | | resection |
| SD0346 | axillary soft | 5 | cm | excisional |
| | tissue | | | biopsy |
| SD6336 | gluteal lesion | not documented | | resection |
| SD1494 | parietal mass | 2.1 | cm | resection |
| SD2056 | lung metastasis | 1.5 | cm | resection |
| SD2051 | groin lymph nodes | 0.5 to 3 | cm | resection |
| SD5038 | upper back lesion | 6.5 | cm | excisional |
| | | | | biopsy |
| SD5934 | abdominal tumor | 4.5 | cm | excisional |
| | nodules | | | biopsy |
| SD5118 | elbow lesion | 3 | cm | excisional |
| | | | | biopsy |
| SD6494 | small bowel | 10 | cm | resection |
| | metastasis | | | |
| SD7357 | skin and breast | 12 | cm | resection |
| | metastasis | | | |
| NR3156 | gluteal lesion | 1.4 | mm | excisional |
| | | | | biopsy |
| NR5784 | axillary lymph | 2 | cm | resection |
| | nodes | | | |
| NR8727 | axillary lymph | 0.2 to 2.2 | cm | resection |
| | nodes | | | |
| NR4949 | parietal metastasis | 1.2 | cm | resection |
| NR1867 | groin lymph nodes | 0.3 to 4 | cm | resection |
| NR3549 | inguinal lymph nodes | 3.2 | cm | excisional |
| | | | | biopsy |
| NR9341 | skin nodules | 1.2 | cm | resection |
| NR4810 | small bowel | 4.5 × 3.5 × 3 | cm | resection |
| | metastasis | | | |

### EXAMPLE 3

### In Vitro Analyses of Immunogenic Peptides

This example demonstrates the in vitro validation of immunogenic peptides.

Translation of next generation sequencing into in vitro validation of peptide predictions has proven challenging even in expert hands, with very low published validation rates.²⁴ In vitro assays are hampered by the paucity of patient material, the sensitivity of preserved cells to the freeze/thaw process, the low frequency of anti-neoantigen T cells within patient material, and the very low sensitivity of T cells in vitro in the absence of the complex in vivo immunogenic microenvironment.

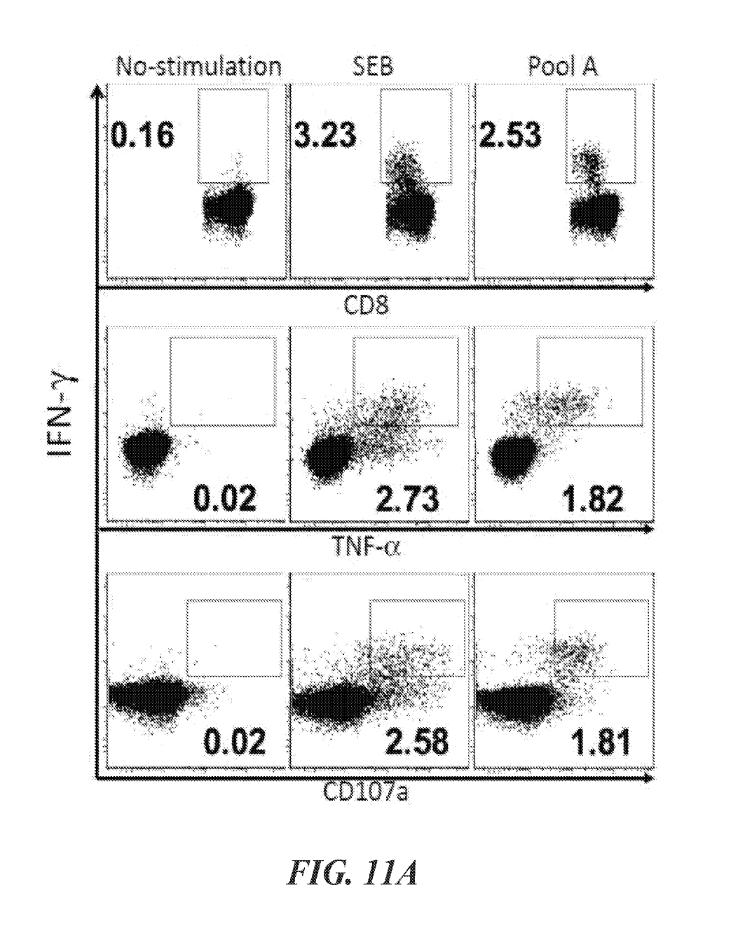

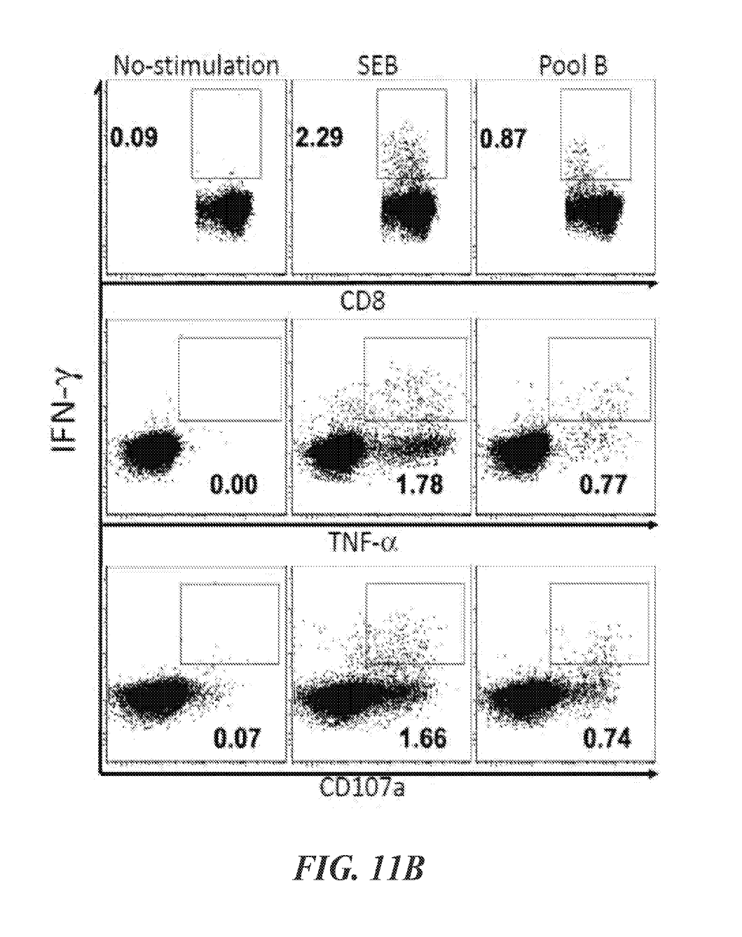

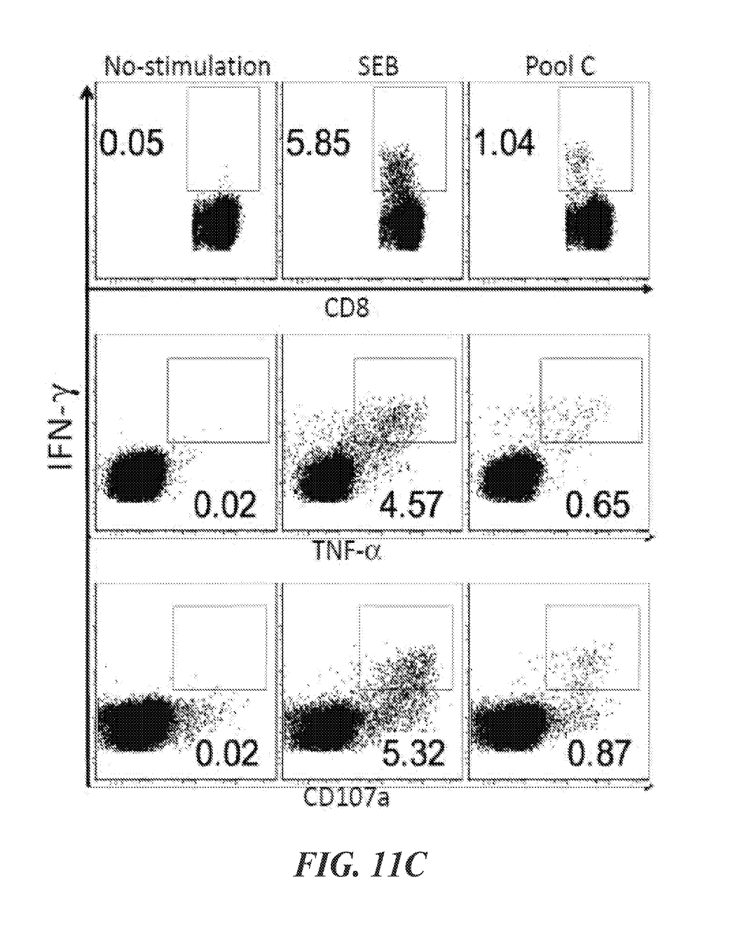

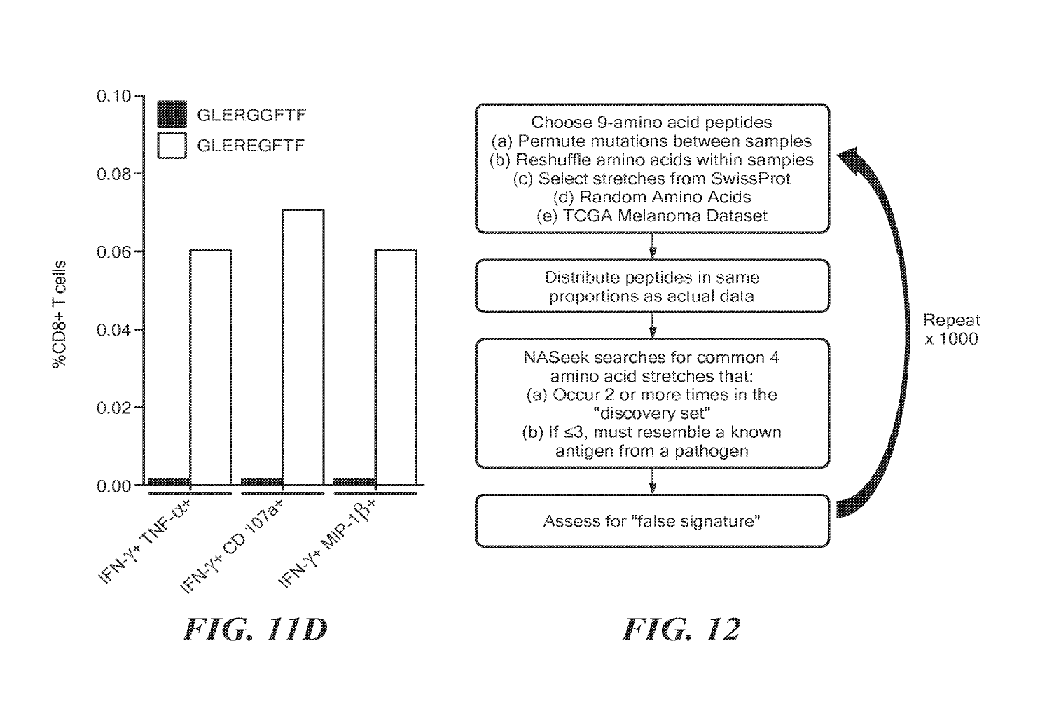

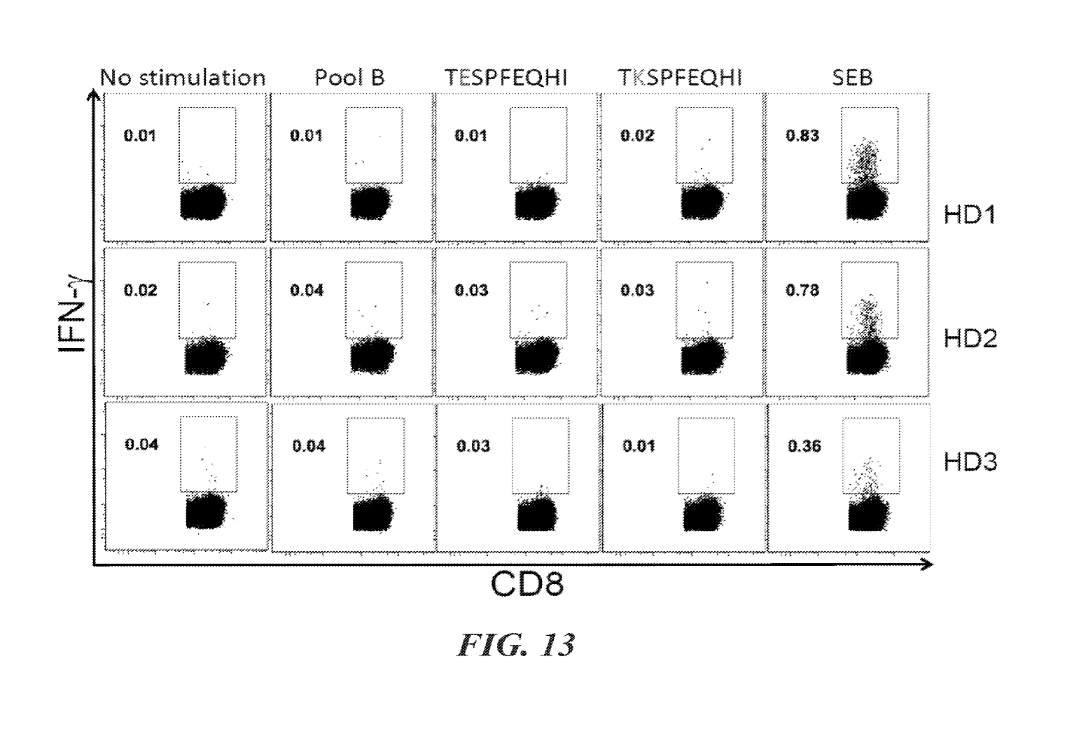

Our system attempted to optimize prediction by integrating multiple high-throughput approaches (FIG. 8). Based on our prediction algorithm, we generated pools of peptides and performed T-cell activation assays for patients for whom we had sufficient lymphocytes (see Methods). Positives pools were observed for 3 of 5 patients (FIG. 11A-C). We identified the exact peptides for patients with adequate peripheral blood mononuclear cells (PBMCs). We found a polyfunctional T cell response to the peptide TESPFEQHI by patient CR9306 (FIG. 4C) as compared to its wild type counterpart TKSPFEQHI. This response peaked at 60 weeks after initiating treatment (FIG. 4D). T-cell responses were absent from healthy donors (FIG. 13). This peptide had a predicted MHC Class I affinity for B4402 of 472 nM, as compared to 18323 nM for TKSPFEQHI. ESPF is a common tetrapeptide found in the response signature, and is a substring (positions 176-179) of the Hepatitis D virus large delta epitope p27 (PESPFA and ESPFAR).^{53,54} TESPFEQHI results from a mutation in FAM3C (c.A577G; p.K193E), a gene highly expressed in melanoma.

We also found that peptide GLEREGFTF elicited a polyfunctional T cell response in patient CR0095 (FIG. 4E and FIG. 11D), as compared to wild type GLERGGFTF. This response peaked at 24 weeks post treatment (FIG. 4E). GLEREGFTF arises from a mutation in CSMD1 (c.G10337A; p.G3446E), which is also highly expressed in melanoma and has 80% homology to a known Burkholderhia pseudomallei antigen (IEDB Reference ID: 1027043). Importantly, the lack of T cell activation may not rule out a given neoantigen as in vitro assays are all limited in sensitivity as described above.

### EXAMPLE 4

### Materials and Methods for Examples 1-3

The present example provides detailed Materials & Methods for the work presented herein in examples 1-3.

We obtained tumor tissue from melanoma patients who were treated with ipilimumab. These samples were from ipilimumab-treated patients who experienced a long term benefit (LB), or minimal/no benefit (NB). Whole exome sequencing was performed on these tumors and matching normal blood. Somatic mutations and candidate somatic neoantigens generated from these mutations were identified and characterized.

### Patient Data

Charts were reviewed independently by two investigators to assign the clinical subgroup and other parameters for discovery and validation sets. Overall survival was calculated as the difference between date of death or censure and first dose of anti-CTLA4 therapy (ipilimumab in the discovery set or ipilimumab or tremelimumab in the validation set). All patients in the discovery set had stage IV melanoma and were treated between 2006 and 2012; samples were collected between 2007 and 2012. Patients in the validation set were treated from 2006 to 2013, and samples were collected between 2005 and 2013. Patients were treated either with commercial ipilimumab (Yervoy) or on clinical trials, including NCT00796991, NCT00495066, NCT00920907, NCT00324155, NCT00162123, NCT0140045, NCT00289640; NCT00495066, NCT00636168, NCT01515189, NCT00086489, and NCT00471887. Patients received varied doses and regimens of ipilimumab, at 3 or 10 mg/kg, and 2 patients were co-treated with dacarbazine or vemurafenib (see FIG. 17). Four patients in the validation set were treated with tremelimumab at a dose of 10 mg/kg×6 (1 patient) or 15 mg/kg×4 (3 patients). Three out of these 4 patients had stage IIIC disease; all other patients included had stage M1a-c. Patients were included who had DNA isolated from frozen tissue for analysis, received at least 2 doses of ipilimumab and had one radiographic assessment at least 12 weeks after first treatment. Two patients in the LB group had an isolated lesion resected in order to render them disease-free. One progressing lesion (CR7623) was sequenced in the training set. In the validation set, 8 tumors represent the non-responding lesions from patients who otherwise had long-term benefit. These include CRNR4941, LSDNR1650, CRNR2472, LSDNR1120, CRNR0244, LSDNR9298, LSDNR3086, and PRO3803. All tumors that progressed undergo molecular analysis as “no benefit” tumors.

Patient data generated in the study has been assembled into a series of tables detailing the following: clinical characteristics of patients in the validation set; detailed clinical characteristics of patients in the discovery set; the discovery set mutation list; loci for which predicted peptide resulting from mutation has a binding affinity of less than 500 nm by NetMHCv3.4; TCGA RNASeq for signature; context, genes and loci for tetrapeptides in the response signature; validation set mutation list; HLA types, discovery and validation sets; and sample site, size, and type.

### DNA Isolation and Whole Exome Sequencing

Primary tumor samples and matched normal specimens (peripheral blood) were obtained with written informed consent per approved institutional review board (IRB) protocols. All specimens were excisional biopsies or resections of clearly visible lesions. All specimens contained high tumor cellularity. Specimens were snap frozen in liquid nitrogen after surgical resection or biopsy and stored at −80° C. Sections stained with hematoxylin and eosin were prepared, and diagnosis was confirmed by a dermatopathologist. DNA was extracted using QIAamp DNA mini kit and QIAamp DNA blood mini kit (Qiagen).

Exon capture was performed using the SureSelect Human All Exon 50MB kit (Agilent). Enriched exome libraries were sequenced on the HiSeq 2000 platform (Illumina) to >100× coverage (MSKCC Genomics Core and Broad Institute, Cambridge, Mass.). Alignment, base-quality score recalibration and duplicate-read removal were performed, germline variants were excluded, mutations annotated and indels evaluated as previously described (FIG. 9A).⁷⁰ Samples with tumor coverage ≦10× were excluded. Medium-confidence reads (11-34×) were manually reviewed using the Integrated Genomics Viewer (IGV) v2.1.⁷¹ Validation rate for sequencing of candidate mutations was 97% for coverage of 10× and above.⁷⁰ Median number of mutations between clinical groups were compared using the Fisher's test.

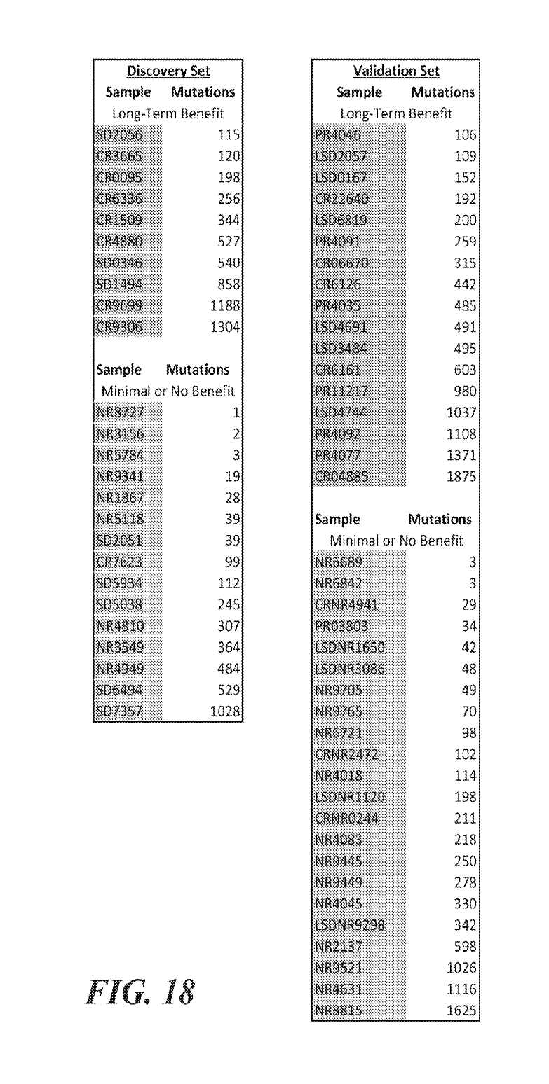

TCGA RNASeq gene expression was normalized by RSEM and mean expression calculated for tumors expressing that gene (see FIG. 18).

### HLA Typing

HLA typing was performed at MSKCC HLA typing lab or New York Blood Center by either low to intermediate resolution polymerase chain reaction-sequence-specific primer (PCR-SSP) method or by high-resolution SeCore HLA sequence-based typing method (HLA-SBT) (Invitrogen). ATHLATES (http://www.braodinstitute.org/scientific-community/science/projects/viral-genomics/athlates)⁷² was also used for HLA typing and confirmation.

### Immunogenicity Analysis

A bioinformatic tool, called NAseek, was created. This program performs two functions: translation of stretches surrounding each mutation, and comparison between the resulting peptides for homology. First, NAseek translated all mutations in exomes so strings of 17 amino acids were generated for the predicted wild type and mutant, with the amino acid resulting from the mutation situated centrally. To evaluate MHC Class I binding, wild type and mutant nonamers containing the tetrapeptides common to the complete responders were input into NetMHC v3.4 (http://www.cbs.dtu.dk/services/NetMHC/) or RANKPEP (http://imed.med.ucm.es/Tools/rankpep.html) for patient-specific HLA types, using a sliding window method. We used a sliding window method as well as locations of altered amino acids in nonapeptides. These programs generated a predicted MHC Class I binding strength. The nonamers that were predicted to be presented by patient-specific MHC Class I were then assessed for similarity to each other. The logo plot of the amino acid frequencies was executed using Weblogo (http://weblogo.berkeley.edu/logo.cgi) with default parameters. The height of letters reflects the relative frequency of the corresponding amino acid at that position. In order to further narrow down the predicted nonamers for testing in vitro, nonamers were also evaluated for putative binding to the T cell receptor using the IEDB immunogenicity predictor with patient-specific HLA types (http://tools.immuneepitope.org/immunogenicity/) or CTLPred (http://www.imtech.res.in/raghava/ctlpred/).

To evaluate T cell activation and homology to known pathogens' antigens, conserved tetrapeptides were analyzed using Immune Epitope Database (www.iedb.org) and assessed as substrings of immunogens in the database for a positive T cell response in Homo sapiens host. We excluded peptides with no predicted T cell response or exclusively anti-self or allergen properties. “Neoantigen signatures” were generated from the nonamers containing the peptides common to patients with long-term benefit (see Table 4 and FIG. 19). A chi-squared test for the total number of shared tetrapeptides was conducted for the LB group relative to the NB group. Standard methods for signature derivation using unsupervised hierarchical clustering followed by logistic regression were used to determine predictive models based solely on the discovery set data. The models were based on the core rule that all tetrapeptides must be present at least twice in the discovery set, and any tetrapeptide present fewer than three times must comprise a common substring of a known antigen shown in vitro to elicit a T cell response. The best fit signature was then applied to the validation set.

We performed rigorous simulation/permutation testing to demonstrate that the neoantigen signature was highly unlikely to result from chance. To assess the null hypothesis that the signature found was due to chance, 5 distinct simulation models were evaluated, three with new datasets and two using permutations of our dataset. The simulations were executed using (a) nonamers drawn from the SwissProt database (b) mutations from the TCGA melanoma dataset (c) randomly generated nonamers (d) redistribution of the mutations found in our data and (e) reordering of the 9 amino acids within each nonamer predicted to be presented in our dataset. In each simulation, the nonamers were distributed randomly, and in proportion to our data (for example, if an actual sample harbored 150 nonamers predicted to bind MHC Class I, then the “virtual” sample was assigned 150 nonamers). Simulation testing was then conducted by applying the same iterative model used on the actual data applied to this virtual dataset, and repeating this process 1,000 times, recording the frequency of signatures greater than the actual signature to determine the p value. P value was calculated as the proportion of iterations with a signature greater that correctly classified segregation of the clinical cohorts, divided by the 1,000 iterations.

Intracellular Cytokine Staining (ICS)

Peripheral blood mononuclear cells (PBMCs) from 5 melanoma patients treated with ipilimumab were collected at multiple time points under IRB-approved institutional protocols. Candidate neoantigen peptides for these patients identified from whole exome/transcriptome analysis were synthesized (GenScript Piscataway, N.J.). 2.5×10⁶ patient PBMC samples were cultured with 2.5×10⁶ irradiated autologous PBMCs pulsed with pools of 30 to 50 peptides per pool in 10% pool human serum (PHS) RPMI 1640 media supplemented with cytokines IL-15 (10 ng/ml) and IL-2 (10 IU/ml). Media was replaced every other day and cells were harvested at day 10.⁷³ The cells were restimulated with the addition of neoantigen peptides in the presence of Brefeldin A and monensin (BD Bioscience) for 6 hours. Cells were then stained with the following antibodies: Pacific Blue-CD3 (clone OKT3), APC-AF750-CD8 (clone SK1, eBioscience) and ECD-CD4 (clone SFC12T4D11, Beckman Coulter). Upon subsequent washing and permeabilizing, the cells were stained with the following antibodies: PE-Cy5-CD107a (clone H4A3), APC-IL-2 (clone MQ1-17H12) PE-MIP-1β (clone D21-1351), FITC-IFN-γ (clone B27) (BD Pharmingen) and PE-Cy7-TNF-α (clone MAB11 eBioscience). Data was acquired using a CYAN flow cytometer and Summit software (Dako Cytomation California Inc., Carpinteria, Calif.). Flow analysis was performed using FlowJo software v9.7.5 (TreeStar, Inc.). When feasible, pools that led to the induction of a cytokine response relative to the no stimulation control were deconvoluted into their component individual peptides. The above process was repeated for the individual peptides and compared to the corresponding predicted wild type nonamer. Staphylococcal enterotoxin B (SEB) served as a positive control for T cell responses.

Immunohistochemistry

Immunohistochemical and hematoxylin and eosin stained slides were scanned using an Aperio slide scanner. Following identification of all necrotic areas contained on the slide, the percent tumor necrosis was determined using Aperio imaging software. Immunostained slides were blindly quantitated by a dermatopathologist using Aperio image analysis algorithms (nuclear and cytoplasmic v9) manually calibrated and verified for each case. A minimum of 3000 cells were counted per case representing the sum of three representative regions with results reported as immunostain positive cells per total cells counted with counting limited to areas of tumor. Sections were stained with the antibodies to the following: LCA (1 ng/μl, DAKO, Clone2B11+PD7/26), CD8 (0.5 ng/μl, DAKO, Clone C8/144B) and Foxp3 (2.5 ng/μl, Abcam, Clone 236A/E7).

Statistical Methods

Mann-Whitney test was used to compare nonsynonymous exonic mutational burden between clinical groups (LB and NB in the discovery and validation sets, respectively). Log-Rank test was used to compare the Kaplan-Meier curves for overall survival in the discovery and validation sets. As described above, simulation testing was used with the null hypothesis that all tetrapeptides contribute equally to clinical benefit to determine if a signature of the size we found happened by chance.

### EXAMPLE 5

### Treatment with Ipilumimab

This example provides instructions treatment of a cancer (melanoma) with an antibody immunotherapy (ipilumimab), as approved by the U.S. Food & Drug Administration for the treatment of metastatic melanoma. In some embodiments, long term clinical benefit is observed after ipilumimab treatment. In accordance with the present invention, the protocol set forth in this example may, in some embodiments, desirably be administered to one or more subjects identified as having a somatic mutation.

YERVOY™ (ipilimumab) Injection, for intravenous infusion Initial United States Approval: 2011

Warning: Immune-Mediated Adverse Reactions

See Full Prescribing Information for Complete Boxed Warning.

YERVOY can result in severe and fatal immune-mediated adverse reactions due to T-cell activation and proliferation. These immune-mediated reactions may involve any organ system; however, the most common severe immune-mediated adverse reactions are enterocolitis, hepatitis, dermatitis (including toxic epidermal necrolysis), neuropathy, and endocrinopathy. The majority of these immune-mediated reactions initially manifested during treatment; however, a minority occurred weeks to months after discontinuation of YERVOY.

Permanently discontinue YERVOY and initiate systemic high-dose corticosteroid therapy for severe immune-mediated reactions. (2.2)

Assess patients for signs and symptoms of enterocolitis, dermatitis, neuropathy, and endocrinopathy and evaluate clinical chemistries including liver function tests and thyroid function tests at baseline and before each dose. (5.1, 5.2, 5.3, 5.4, 5.5)

### Indications and Usage

YERVOY is a human cytotoxic T-lymphocyte antigen 4 (CTLA-4)-blocking antibody indicated for the treatment of unresectable or metastatic melanoma. (1)

### Dosage and Administration

YERVOY 3 mg/kg administered intravenously over 90 minutes every 3 weeks for a total of four doses. (2.1)
Permanently discontinue for severe adverse reactions. (2.2)

Full Prescribing Information

Warning: Immune-Mediated Adverse Reactions

YERVOY can result in severe and fatal immune-mediated adverse reactions due to T-cell activation and proliferation. These immune-mediated reactions may involve any organ system; however, the most common severe immune-mediated adverse reactions are enterocolitis, hepatitis, dermatitis (including toxic epidermal necrolysis), neuropathy, and endocrinopathy. The majority of these immune-mediated reactions initially manifested during treatment; however, a minority occurred weeks to months after discontinuation of YERVOY.

Permanently discontinue YERVOY and initiate systemic high-dose corticosteroid therapy for severe immune-mediated reactions. [See Dosage and Administration (2.2)]

Assess patients for signs and symptoms of enterocolitis, dermatitis, neuropathy, and endocrinopathy and evaluate clinical chemistries including liver function tests and thyroid function tests at baseline and before each dose. [See Warnings and Precautions (5.1, 5.2, 5.3, 5.4, 5.5)]

1 Indications and Usage

YERVOY (ipilimumab) is indicated for the treatment of unresectable or metastatic melanoma.

2 Dosage and Administration

2.1 Recommended Dosing

The recommended dose of YERVOY is 3 mg/kg administered intravenously over 90 minutes every 3 weeks for a total of four doses.

2.2 Recommended Dose Modifications
Withhold scheduled dose of YERVOY for any moderate immune-mediated adverse reactions or for symptomatic endocrinopathy. For patients with complete or partial resolution of adverse reactions (Grade 0-1), and who are receiving less than 7.5 mg prednisone or equivalent per day, resume YERVOY at a dose of 3 mg/kg every 3 weeks until administration of all 4 planned doses or 16 weeks from first dose, whichever occurs earlier.

Permanently discontinue YERVOY for any of the following:
Persistent moderate adverse reactions or inability to reduce corticosteroid dose to 7.5 mg prednisone or equivalent per day.
Failure to complete full treatment course within 16 weeks from administration of first dose.
Severe or life-threatening adverse reactions, including any of the following:

Colitis with abdominal pain, fever, ileus, or peritoneal signs; increase in stool frequency (7 or more over baseline), stool incontinence, need for intravenous hydration for more than 24 hours, gastrointestinal hemorrhage, and gastrointestinal perforation

Aspartate aminotransferase (AST) or alanine aminotransferase (ALT) >5 times the upper limit of normal or total bilirubin >3 times the upper limit of normal

Stevens-Johnson syndrome, toxic epidermal necrolysis, or rash complicated by full thickness dermal ulceration, or necrotic, bullous, or hemorrhagic manifestations

Severe motor or sensory neuropathy, Guillain-Barré syndrome, or myasthenia gravis

Severe immune-mediated reactions involving any organ system (eg, nephritis, pneumonitis, pancreatitis, non-infectious myocarditis)

Immune-mediated ocular disease that is unresponsive to topical immunosuppressive therapy

2.3 Preparation and Administration
Do not shake product.
Inspect parenteral drug products visually for particulate matter and discoloration prior to administration. Discard vial if solution is cloudy, there is pronounced discoloration (solution may have pale yellow color), or there is foreign particulate matter other than translucent-towhite, amorphous particles.

Preparation of Solution
Allow the vials to stand at room temperature for approximately 5 minutes prior to preparation of infusion.
Withdraw the required volume of YERVOY and transfer into an intravenous bag.
Dilute with 0.9% Sodium Chloride Injection, USP or 5% Dextrose Injection, USP to prepare a diluted solution with a final concentration ranging from 1 mg/mL to 2 mg/mL. Mix diluted solution by gentle inversion.
Store the diluted solution for no more than 24 hours under refrigeration (2° C. to 8° C., 36° F. to 46° F.) or at room temperature (20° C. to 25° C., 68° F. to 77° F.).
Discard partially used vials or empty vials of YERVOY.

Administration Instructions
Do not mix YERVOY with, or administer as an infusion with, other medicinal products.
Flush the intravenous line with 0.9% Sodium Chloride Injection, USP or 0.5% Dextrose Injection, USP after each dose.
Administer diluted solution over 90 minutes through an intravenous line containing a sterile, non-pyrogenic, low-protein-binding in-line filter.

3 Dosage Forms and Strengths

50 mg/10 mL (5 mg/mL). 200 mg/40 mL (5 mg/mL).

4 Contraindications

None.

5 Warnings and Precautions

YERVOY can result in severe and fatal immune-mediated reactions due to T-cell activation and proliferation.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Hodi F S, O'Day S J, McDermott D F, et al. Improved survival with ipilimumab in patients with metastatic melanoma. N Engl J Med 2010; 363:711-23.
2. Wolchok J D, Kluger H, Callahan M K, et al. Nivolumab plus ipilimumab in advanced melanoma. N Engl J Med 2013; 369:122-33.
3. Prieto P A, Yang J C, Sherry R M, et al. CTLA-4 blockade with ipilimumab: long-term follow-up of 177 patients with metastatic melanoma. Clin Cancer Res 2012; 18:2039-47.
4. Hamid O, Robert C, Daud A, et al. Safety and tumor responses with lambrolizumab (anti-PD-1) in melanoma. N Engl J Med 2013; 369:134-44.
5. Topalian S L, Hodi F S, Brahmer J R, et al. Safety, activity, and immune correlates of anti-PD-1 antibody in cancer. N Engl J Med 2012; 366:2443-54.
6. Postow M A, Callahan M K, Wolchok J D. The antitumor immunity of ipilimumab: (T-cell) memories to last a lifetime? Clin Cancer Res 2012; 18:1821-3.
7. Carthon B C, Wolchok J D, Yuan J, et al. Preoperative CTLA-4 blockade: tolerability and immune monitoring in the setting of a presurgical clinical trial. Clin Cancer Res 2010; 16:2861-71.
8. Ku G Y, Yuan J, Page D B, et al. Single-institution experience with ipilimumab in advanced melanoma patients in the compassionate use setting: lymphocyte count after 2 doses correlates with survival. Cancer 2010; 116:1767-75.
9. Ji R R, Chasalow S D, Wang L, et al. An immune-active tumor microenvironment favors clinical response to ipilimumab. Cancer Immunol Immunother 2012; 61:1019-31.
10. Harlin H, Meng Y, Peterson A C, et al. Chemokine expression in melanoma metastases associated with CD8+ T-cell recruitment. Cancer Res 2009; 69:3077-85.
11. Gajewski T F, Louahed J, Brichard V G. Gene signature in melanoma associated with clinical activity: a potential clue to unlock cancer immunotherapy. Cancer J 2010; 16:399-403.
12. Hamid O, Schmidt H, Nissan A, et al. A prospective phase II trial exploring the association between tumor microenvironment biomarkers and clinical activity of ipilimumab in advanced melanoma. J Transl Med 2011; 9:204.
13. Castle J C, Kreiter S, Diekmann J, et al. Exploiting the mutanome for tumor vaccination. Cancer Res 2012; 72:1081-91.
14. Srivastava N, Srivastava P K. Modeling the repertoire of true tumor-specific MHC I epitopes in a human tumor. PLoS One 2009; 4:e6094.
15. Peggs K S, Quezada S A, Chambers C A, Korman A J, Allison J P. Blockade of CTLA-4 on both effector and regulatory T cell compartments contributes to the antitumor activity of anti-CTLA-4 antibodies. J Exp Med 2009; 206:1717-25.
16. Chambers C A, Kuhns M S, Allison J P. Cytotoxic T lymphocyte antigen-4 (CTLA-4) regulates primary and secondary peptide-specific CD4(+) T cell responses. Proc Natl Acad Sci USA 1999; 96:8603-8.
17. Chambers C A, Sullivan T J, Truong T, Allison J P. Secondary but not primary T cell responses are enhanced in CTLA-4-deficient CD8+ T cells. Eur J Immunol 1998; 28:3137-43.
18. Simpson T R, Li F, Montalvo-Ortiz W, et al. Fc-dependent depletion of tumor-infiltrating regulatory T cells co-defines the efficacy of anti-CTLA-4 therapy against melanoma. J Exp Med 2013.
19. Alexandrov L B, Nik-Zainal S, Wedge D C, et al. Signatures of mutational processes in human cancer. Nature 2013; 500:415-21.
20. Lawrence M S, Stojanov P, Polak P, et al. Mutational heterogeneity in cancer and the search for new cancer-associated genes. Nature 2013; 499:214-8.
21. Segal N H, Parsons D W, Peggs K S, et al. Epitope landscape in breast and colorectal cancer. Cancer Res 2008; 68:889-92.
22. Dunn G P, Old L J, Schreiber R D. The immunobiology of cancer immunosurveillance and immunoediting. Immunity 2004; 21:137-48.
23. Matsushita H, Vesely M D, Koboldt D C, et al. Cancer exome analysis reveals a T-cell-dependent mechanism of cancer immunoediting. Nature 2012; 482:400-4.
24. van Rooij N, van Buuren M M, Philips D, et al. Tumor Exome Analysis Reveals Neoantigen-Specific T-Cell Reactivity in an Ipilimumab-Responsive Melanoma. J Clin Oncol 2013.
25. Tran E, Turcotte S, Gros A, et al. Cancer immunotherapy based on mutation-specific CD4+ T cells in a patient with epithelial cancer. Science 2014; 344:641-5.
26. Wolchok J D, Weber J S, Hamid O, et al. Ipilimumab efficacy and safety in patients with advanced melanoma: a retrospective analysis of HLA subtype from four trials. Cancer Immun 2010; 10:9.
27. Wolchok J D, Neyns B, Linette G, et al. Ipilimumab monotherapy in patients with pretreated advanced melanoma: a randomised, double-blind, multicentre, phase 2, dose-ranging study. Lancet Oncol 2010; 11:155-64.
28. O'Day S J, Maio M, Chiarion-Sileni V, et al. Efficacy and safety of ipilimumab monotherapy in patients with pretreated advanced melanoma: a multicenter single-arm phase II study. Ann Oncol 2010; 21:1712-7.
29. Robert C, Thomas L, Bondarenko I, et al. Ipilimumab plus dacarbazine for previously untreated metastatic melanoma. N Engl J Med 2011; 364:2517-26.
30. Gartner J J, Parker S C, Prickett T D, et al. Whole-genome sequencing identifies a recurrent functional synonymous mutation in melanoma. Proc Natl Acad Sci USA 2013; 110:13481-6.
31. Furney S J, Turajlic S, Fenwick K, et al. Genomic characterisation of acral melanoma cell lines. Pigment Cell Melanoma Res 2012; 25:488-92.
32. Wei X, Walia V, Lin J C, et al. Exome sequencing identifies GRIN2A as frequently mutated in melanoma. Nat Genet 2011; 43:442-6.
33. Berger M F, Hodis E, Heffernan T P, et al. Melanoma genome sequencing reveals frequent PREX2 mutations. Nature 2012; 485:502-6.
34. Krauthammer M, Kong Y, Ha B H, et al. Exome sequencing identifies recurrent somatic RAC1 mutations in melanoma. Nat Genet 2012; 44:1006-14.
35. Hodis E, Watson I R, Kryukov G V, et al. A landscape of driver mutations in melanoma. Cell 2012; 150:251-63.
36. Almunia C, Bretaudeau M, Held G, et al. Bee Venom Phospholipase A2, a Good “Chauffeur” for Delivering Tumor Antigen to the MHC I and MHC II Peptide-Loading Compartments of the Dendritic Cells: The Case of NY-ESO-1. PLoS One 2013; 8:e67645.
37. Ray S, Chhabra A, Chakraborty N G, et al. MHC-I-restricted melanoma antigen specific TCR-engineered human CD4+ T cells exhibit multifunctional effector and helper responses, in vitro. Clin Immunol 2010; 136:338-47.
38. Stuart G W, Moffett K, Leader J J. A comprehensive vertebrate phylogeny using vector representations of protein sequences from whole genomes. Mol Biol Evol 2002; 19:554-62.
39. Birnbaum M E, Mendoza J L, Sethi D K, et al. Deconstructing the Peptide-MHC Specificity of T Cell Recognition. Cell; 157:1073-87.
40. Morita D, Yamamoto Y, Suzuki J, Mori N, Igarashi T, Sugita M. Molecular requirements for T cell recognition of N-myristoylated peptides derived from the simian immunodeficiency virus Nef protein. J Virol 2013; 87:482-8.
41. Rahman A K, Herfst C A, Moza B, et al. Molecular basis of TCR selectivity, cross-reactivity, and allelic discrimination by a bacterial superantigen: integrative functional and energetic mapping of the SpeC-Vbeta2.1 molecular interface. J Immunol 2006; 177:8595-603.
42. Binkowski T A, Marino S R, Joachimiak A. Predicting HLA class I non-permissive amino acid residues substitutions. PLoS One 2012; 7:e41710.
43. Piepenbrink K H, Blevins S J, Scott D R, Baker B M. The basis for limited specificity and MHC restriction in a T cell receptor interface. Nat Commun 2013; 4:1948.
44. Aleksic M, Dushek O, Zhang H, et al. Dependence of T cell antigen recognition on T cell receptor-peptide MHC confinement time. Immunity 2010; 32:163-74.
45. Insaidoo F K, Borbulevych O Y, Hossain M, Santhanagopolan S M, Baxter T K, Baker B M. Loss of T cell antigen recognition arising from changes in peptide and major histocompatibility complex protein flexibility: implications for vaccine design. J Biol Chem 2011; 286:40163-73.
46. Sliz P, Michielin O, Cerottini J C, et al. Crystal structures of two closely related but antigenically distinct HLA-A2/melanocyte-melanoma tumor-antigen peptide complexes. J Immunol 2001; 167:3276-84.
47. Cong H, Mui E J, Witola W H, et al. Human immunome, bioinformatic analyses using HLA supermotifs and the parasite genome, binding assays, studies of human T cell responses, and immunization of HLA-A*1101 transgenic mice including novel adjuvants provide a foundation for HLA-A03 restricted CD8+T cell epitope based, adjuvanted vaccine protective against Toxoplasma gondii. Immunome Res 2010; 6:12.
48. Tan T G, Mui E, Cong H, et al. Identification of T. gondii epitopes, adjuvants, and host genetic factors that influence protection of mice and humans. Vaccine 2010; 28:3977-89.
49. Wong R, Lau R, Chang J, et al. Immune responses to a class II helper peptide epitope in patients with stage III/IV resected melanoma. Clin Cancer Res 2004; 10:5004-13.
50. Ekeruche-Makinde J, Clement M, Cole D K, et al. T-cell receptor-optimized peptide skewing of the T-cell repertoire can enhance antigen targeting. J Biol Chem 2012; 287:37269-81.
51. Li Y, Depontieu F R, Sidney J, et al. Structural basis for the presentation of tumor-associated MHC class II-restricted phosphopeptides to CD4+ T cells. J Mol Biol 2010; 399:596-603.
52. Voelter V, Rufer N, Reynard S, et al. Characterization of Melan-A reactive memory CD8+ T cells in a healthy donor. Int Immunol 2008; 20:1087-96.
53. Wang J G, Jansen R W, Brown E A, Lemon S M. Immunogenic domains of hepatitis delta virus antigen: peptide mapping of epitopes recognized by human and woodchuck antibodies. J Virol 1990; 64:1108-16.
54. Poisson F, Baillou F, Dubois F, Janvier B, Roingeard P, Goudeau A. Immune response to synthetic peptides of hepatitis delta antigen. J Clin Microbiol 1993; 31:2343-9.
55. Monach P A, Meredith S C, Siegel C T, Schreiber H. A unique tumor antigen produced by a single amino acid substitution. Immunity 1995; 2:45-59.
56. Dubey P, Hendrickson R C, Meredith S C, et al. The immunodominant antigen of an ultraviolet-induced regressor tumor is generated by a somatic point mutation in the DEAD box helicase p68. J Exp Med 1997; 185:695-705.
57. Noguchi Y, Chen Y T, Old L J. A mouse mutant p53 product recognized by CD4+ and CD8+ T cells. Proc Natl Acad Sci USA 1994; 91:3171-5.
58. Ikeda H, Ohta N, Furukawa K, et al. Mutated mitogen-activated protein kinase: a tumor rejection antigen of mouse sarcoma. Proc Natl Acad Sci USA 1997; 94:6375-9.
59. Matsutake T, Srivastava PK. The immunoprotective MHC II epitope of a chemically induced tumor harbors a unique mutation in a ribosomal protein. Proc Natl Acad Sci USA 2001; 98:3992-7.
60. Allen P M, Matsueda G R, Evans R J, Dunbar J B, Jr., Marshall G R, Unanue E R. Identification of the T-cell and Ia contact residues of a T-cell antigenic epitope. Nature 1987; 327:713-5.
61. Abrahmsen L, Dohlsten M, Segren S, Bjork P, Jonsson E, Kalland T. Characterization of two distinct MHC class II binding sites in the superantigen staphylococcal enterotoxin A. EMBO J 1995; 14:2978-86.
62. Sant'Angelo D B, Robinson E, Janeway C A, Jr., Denzin L K. Recognition of core and flanking amino acids of MHC class II-bound peptides by the T cell receptor. Eur J Immunol 2002; 32:2510-20.
63. Anderson M W, Gorski J. Cutting edge: TCR contacts as anchors: effects on affinity and HLA-DM stability. J Immunol 2003; 171:5683-7.
64. Donermeyer D L, Weber K S, Kranz D M, Allen P M. The study of high-affinity TCRs reveals duality in T cell recognition of antigen: specificity and degeneracy. J Immunol 2006; 177:6911-9.
65. Postow M A, Luke J J, Bluth M J, et al. Ipilimumab for patients with advanced mucosal melanoma. Oncologist 2013; 18:726-32.
66. Luke J J, Callahan M K, Postow M A, et al. Clinical activity of ipilimumab for metastatic uveal melanoma: A retrospective review of the Dana-Farber Cancer Institute, Massachusetts General Hospital, Memorial Sloan-Kettering Cancer Center, and University Hospital of Lausanne experience. Cancer 2013; 119:3687-95.
67. Del Vecchio M, Di Guardo L, Ascierto P A, et al. Efficacy and safety of ipilimumab 3 mg/kg in patients with pretreated, metastatic, mucosal melanoma. Eur J Cancer 2013.
68. Srivastava P K, Duan F. Harnessing the antigenic fingerprint of each individual cancer for immunotherapy of human cancer: genomics shows a new way and its challenges. Cancer Immunol Immunother 2013; 62:967-74.
69. Cha E, Klinger M, Hou Y, et al. Improved survival with T cell clonotype stability after anti-CTLA-4 treatment in cancer patients. Sci Transl Med 2014; 6:238ra70.
70. Ho A S, Kannan K, Roy D M, et al. The mutational landscape of adenoid cystic carcinoma. Nat Genet 2013; 45:791-8.
71. Robinson J T, Thorvaldsdottir H, Winckler W, et al. Integrative genomics viewer. Nat Biotechnol 2011; 29:24-6.
72. Liu C, Yang X, Duffy B, et al. ATHLATES: accurate typing of human leukocyte antigen through exome sequencing. Nucleic Acids Res 2013; 41:e142.
73. Lin Y, Gallardo H F, Ku G Y, et al. Optimization and validation of a robust human T-cell culture method for monitoring phenotypic and polyfunctional antigen-specific CD4 and CD8 T cell responses. Cytotherapy 2009; 11:912-22.

We claim:

1. A method comprising steps of:
detecting a somatic mutation in a cancer sample from a subject; and
identifying the subject as a candidate for treatment with an immune checkpoint modulator.

2. The method of claim 1 wherein the step of detecting comprises sequencing one or more exomes from the cancer sample.

3. The method of claim 1 wherein the somatic mutation comprises a neoepitope recognized by a T cell.

4. The method of claim 2 wherein the neoepitope has greater binding affinity to a major histocompatibility complex (MHC) molecule compared to a corresponding epitope that does not have a mutation.

5. The method of claim 1 wherein the somatic mutation comprises a neoepitope comprising a tetramer that is not expressed in the same cell type that does not have a somatic mutation.

6. The method of claim 5 wherein the neoepitope shares a consensus sequence with an infectious agent.

7. The method of claim 5 wherein the tetramer is a sequence selected from those presented in Table 1.

8. The method of claim 1 wherein the cancer is or comprises a melanoma.

9. The method of claim 1 wherein the immune checkpoint modulator interacts with cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof.

10. The method of claim 1 wherein the immune checkpoint modulator is an antibody agent.

11. The method of claim 10, wherein the antibody agent is or comprises a monoclonal antibody or antigen binding fragment thereof.

12. The method of claim 11 wherein the antibody is ipilumimab.

13. The method of claim 1 wherein the subject has not previously been treated with a cancer therapeutic.

14. The method of claim 1 wherein the subject has not previously been treated with a cancer immunotherapeutic.

15. The method of claim 12, further comprising a step of administering ipilumimab to the subject.

16. A method comprising steps of:
detecting a somatic mutation in a cancer sample from a subject; and
identifying the subject as a poor candidate for treatment with an immune checkpoint modulator.

17. The method of claim 16 wherein the subject is identified as likely to suffer one or more autoimmune complications if administered an immune checkpoint modulator.

18. The method of claim 17 wherein the autoimmune complication is hypothyroidism.

19. A method comprising steps of:
determining a subject has a cancer comprising a somatic mutation, wherein the somatic mutation comprises a neoepitope comprising a tetramer from Table 1, and
selecting for the subject a cancer treatment comprising an immune checkpoint modulator.

20. The method of claim 19 wherein the cancer comprises melanoma.

21. The method of claim 19 wherein the immune checkpoint modulator interacts with cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof.

22. The method of claim 21 wherein the immune checkpoint modulator is an antibody agent.

23. The method of claim 22 wherein the antibody agent is or comprises a monoclonal antibody or antigen binding fragment thereof.

24. The method of claim 23 wherein the antibody is ipilumimab.

25. The method of claim 19 wherein the subject has not previously been treated with a cancer therapeutic.

26. The method of claim 19 wherein the subject has not previously been treated with a cancer immunotherapeutic.

27. A method of treating a subject with an immune checkpoint modulator wherein the subject has previously been identified to have a cancer with one or more somatic mutations, wherein the one or more somatic mutations comprises a neoepitope recognized by a T cell.

28. The method of claim 27 wherein the cancer comprises melanoma.

29. The method of claim 27 wherein the immune checkpoint modulator interacts with cytotoxic T-lymphocyte antigen 4 (CTLA4), programmed death 1 (PD-1) or its ligands, lymphocyte activation gene-3 (LAG3), B7 homolog 3 (B7-H3), B7 homolog 4 (B7-H4), indoleamine (2,3)-dioxygenase (IDO), adenosine A2a receptor, neuritin, B- and T-lymphocyte attenuator (BTLA), killer immunoglobulin-like receptors (KIR), T cell immunoglobulin and mucin domain-containing protein 3 (TIM-3), inducible T cell costimulator (ICOS), CD27, CD28, CD40, CD137, or combinations thereof.

30. The method of claim 27 wherein the immune checkpoint modulator is an antibody agent.

31. The method of claim 30 wherein the antibody agent is or comprises a monoclonal antibody or antigen binding fragment thereof.

32. The method of claim 31 wherein the antibody is ipilumimab.

33. The method of claim 27 wherein the subject has not previously been treated with a cancer therapeutic.

34. The method of claim 27 wherein the subject has not previously been treated with a cancer immunotherapeutic.

35. A method of improving efficacy of cancer therapy with an immune checkpoint modulator, the method comprising a step of:
selecting for receipt of the therapy a subject identified as having a cancer with one or more somatic mutations comprising a neoepitope recognized by a T cell.

36. In a method of treating cancer by administering immune checkpoint modulator therapy, the improvement that comprises:
administering the therapy to a subject identified as having a cancer with one or more somatic mutations comprising a neoepitope recognized by a T cell.

37. A method of treating a cancer selected from the group consisting of carcinoma, sarcoma, myeloma, leukemia, or lymphoma, the method comprising a step of:
administering immune checkpoint modulator therapy to a subject identified as having a cancer with one or more somatic mutations comprising a neoepitope recognized by a T cell.

38. The method of claim 37 wherein the cancer is or comprises melanoma.

39. A method of defining a response signature for an immune checkpoint modulator therapy, the method comprising steps of:
comparing genetic sequence information from a first plurality of tumor samples, which first plurality contains samples that share a common response feature to immune checkpoint modulator therapy, with that obtained from a second plurality of tumor samples, which second plurality contains samples that do not share the common response feature but are otherwise comparable to those of the first set, so that the comparison defines genetic sequence elements whose presence is associated or correlates with the common response feature; and
determining which of the defined genetic sequence elements generate a neoepitope; and
defining as a signature for the common response feature presence of the neoepitope.

40. The method of claim 39, further comprising a step of:
determining which of the neoepitopes alters peptide-MHC binding strength,
wherein the step of defining as a signature for the common response feature involves defining as the signature at least one of the neoepitopes determined to alter peptide-MHC biding strength.

41. The method of claim 40, wherein the step of defining as a signature for the common response feature involves defining as the signature a set of the neoepitopes determined to alter peptide-MHC biding strength.

42. The method of any one of claims 39-41, wherein the neoepitope is or comprises a tetramer.

43. The method of claim 42, wherein the neoepitope is or comprises a tetramer set forth in Table 1.

44. The method of claim 44, wherein the set of neoepitopes comprises or consists of a plurality of neoepitopes set forth in Table 1.
that does not share the common response feature analyzing a plurality of tumor samples so that we analyzed tumor and matched blood DNA using whole exome sequencing. In the discovery set, we generated 6.4 GB of mapped sequence, with over 90% of the target sequence covered to at least 10× depth and mean exome coverage of 103× (FIG. 5). The wide range of mutational burdens among samples (FIGS. 2A and 2B) and recurrent mutations (FIG. 6A), were consistent with the literature
We examined whether a subset of somatic neoepitopes would alter the strength of peptide-MHC binding, using patient-specific HLA types. We first compared the overall antigenicity trend of all mutant versus wild type peptides. Intriguingly, in aggregate, the mutant peptides were predicted to bind MHC Class I with higher affinity than the corresponding wild type peptides (FIGS. 10A and 10B).
